(19) United States
(12) Patent Application Publication
(10) Pub. No.: US 2016/0040221 A1
(43) Pub. Date: Feb. 11, 2016

(54) IDENTIFICATION OF BIOLOGICALLY AND CLINICALLY ESSENTIAL GENES AND GENE PAIRS, AND METHODS EMPLOYING THE IDENTIFIED GENES AND GENE PAIRS

(71) Applicant: Agency for Science, Technology and Research, Singapore (SG)

(72) Inventors: Vladimir A. Kuznetsov, Singapore (SG); Efthimios Motakis, Singapore (SG); Anna V. Ivshina, Singapore (SG)

(73) Assignee: Agency for Science, Technology and Research, Singapore (SG)

(21) Appl. No.: 14/301,296

(22) Filed: Jun. 10, 2014

Related U.S. Application Data
(62) Division of application No. 13/255,898, filed on Sep. 9, 2011, now abandoned.
(60) Provisional application No. 61/158,948, filed on Mar. 10, 2009.

(51) Int. Cl. C12Q 1/68

(52) U.S. Cl. CPC C12Q 1/6837

(57) ABSTRACT

A method of obtaining cut-off expression values should be selected so as to maximise the separation of the respective survival curves of the two groups of patients. Pairs of genes are statistically significant genes are generated by generating a plurality of models, each of which represents a way of partitioning a set of subjects based on the optimal cut-off expression values of the pair of genes. Those gene pairs are identified for which one of the models has a high prognostic significance. Novel survival significant gene sets forming functional modules which could be used to develop specific prognostic and predictive tests are derived.

### CROSS-REFERENCE TO RELATED APPLICATIONS

This patent application is a divisional application of U.S. patent application Ser. No. 13/255,898, filed Sep. 9, 2011, entitled IDENTIFICATION OF BIOLOGICALLY AND CLINICALLY ESSENTIAL GENES AND GENE PAIRS, AND METHODS EMPLOYING THE IDENTIFIED GENES AND GENE PAIRS, which is a U.S. National Phase application under 35 U.S.C. §371 of International Application No. PCT/SG2010/000080, filed on Mar. 10, 2010, entitled IDENTIFICATION OF BIOLOGICALLY AND CLINICALLY ESSENTIAL GENES AND GENE PAIRS, AND METHODS EMPLOYING THE IDENTIFIED GENES AND GENE PAIRS, which claims priority to U.S. provisional patent application No. 61/158,948, filed Mar. 10, 2009, and also is related to Singapore patent application number 200901682-5, which has the same filing date as U.S. 61/158,948.

### SEQUENCE LISTING

This application incorporates by reference the material (Sequence Listing) in the ASCII text file Sequence_Listing.txt, created Sep. 9, 2011, having a file size of 99,000 bytes.

### FIELD OF THE INVENTION

The present invention relates to identification of clinically distinct sub-groups of patients and corresponding genes and/or pairs of genes for which the respective gene expression values in a subject and clinical status are statistically significant in relation to a medical condition, for example cancer progression, or more particularly breast cancer patient's survival. The gene expression values may for example be indicative of the susceptibility of the individual subject to the medical condition (in context of time survival or/and disease progression), or the prognosis of a subject who exhibits the medical condition. The invention further relates to methods employing the identified patient survival significant genes and gene pairs.

### BACKGROUND OF THE INVENTION

Global gene expression profiles of subjects are often used to obtain information about those subjects, such as their susceptibility to certain medical condition, or, in the case of subjects exhibiting medical conditions, their prognosis. For example, having determined that a particular gene is important, the level in which that gene is expressed in a subject can be used to classify the individual into one of a plurality of classes, each class being associated with a different susceptibility or prognosis. The class comparison analysis leads to a better understanding of the disease process by identifying gene expression in primary tumours associated with subject survival outcomes (Kuznetsov et al., 2006).

### 1. The Theory of Survival Analysis

First we will describe briefly the background theory of survival analysis. We denote by T the patient's survival time. T is a continuous non-negative random variable which can take values t, tε[0, ∞). T has density function f(t) and cumulative distribution function

$$F ( t ) = P ( T \leq t ) \cdot F ( t ) = {\int }_{0}^{t} f ( {t}^{′} ) d {t}^{′} .$$

We are primarily interested in estimating two quantities:
The survival function: S(t)=P(T>t)=1−F(t)

The hazard function:

$$h ( t ) = \frac{f ( t )}{S ( t )} = {lim}_{Δ t \to 0} \frac{P ( t \leq T < ( t + Δ t ) \vert T \geq t )}{Δ t}$$

The survival function expresses the probability of a patient to be alive at time t. It is often presented in the form S(t)=exp(−H(t)), where

$$H ( t ) = {\int }_{0}^{t} h ( u ) d u$$

denotes the cumulative hazard. The hazard function assesses the instantaneous risk of death at time t, conditional on survival up to that time.

Notice that the hazard function is expressed in terms of the survival function. To this extent, survival distributions and hazard functions can be generated for any distribution defined for tε[0,∞). By considering a random variable W, distributed in (∞,−∞), we can generate a family of survival distributions by introducing location (α) and scale (σ) changes of the form log T=α+σW.

Alternatively, we can express the relationship of the survival distribution to covariates by means of a parametric model. The parametric model employs a “regressor” variable x. Take for example a model based on the exponential distribution and write: log(h(t))=α+βx, or equivalently, h(t)=exp(α+βx).

This is a linear model for the log-hazard, or, equivalently, a multiplicative model for the hazard. The constant α represents the log-baseline hazard (the hazard when the regressor x=0) and the slope parameter β gives the change in hazard rate as x varies. This is an easy example of how survival models can be obtained from simple distributional assumptions. In the next paragraphs we will see more specific examples.

2. Cox Proportional Hazards. Model

One of the most popular survival models is the Cox proportional hazards model (Cox, 1972):

log h(t)=α(t)+βx (1)

where, as before, t is the survival time, h(t) represents the hazard function, α(t) is the baseline hazard, β is the slope parameter of the model and x is the regressor. The popularity of this model lies in the fact that it leaves the baseline hazard function α(t) (which we may alternatively designate as log h₀(t)) unspecified (no distribution assumed). It can be estimated iteratively by the method of partial likelihood of Cox (1972). The Cox proportional hazards model is semi-parametric because while the baseline hazard can take any form, the covariates enter the model linearly.

Cox (1972) showed that the p coefficient can be estimated efficiently by the Cox partial likelihood function. Suppose that for each of a plurality of K subjects (labelled by k=1, . . . , K), we observe at corresponding time tₖ a certain nominal (i.e. yes/no) clinical event has occurred (e.g. whether there has been metastasis). This knowledge is denoted eₖ. For example eₖ may be 0 if the event has not occurred by time tₖ (e.g. no tumour metastasis at time tₖ) and 1 if the event has occurred (e.g. tumour metastasis at time tₖ). Cox (1972) showed that the p coefficient can be estimated efficiently by the Cox partial likelihood function, estimated as:

$$L ( {β}_{i} ) = {\prod }_{k = 1}^{K} {\{ \frac{exp ( β {x}_{k} )}{{\sum }_{j ∈ R ( {t}_{k} )} exp ( β {x}_{j} )} \}}^{{e}_{k}} \qquad ( 2 )$$

where R(tₖ)={j: tⱼ≧tₖ} is the risk set at time tₖ. Typically, e is a binary variable taking value 0=non-occurrence of the event until time t or 1=occurrence of the event at time t. Later we will discuss a particular case of clinical event we consider in the work, without limiting though our model to this specific case.

The likelihood (2) is minimized by Newton-Raphson optimization method for finding successively better approximations to the zeroes (or roots) of a real-valued function (Press et al., 1992), with a very simple elimination algorithm to invert and solve the simultaneous equations.

### 3. The Goodness-of-Split Measure of Survival and Selection of Prognostic Significant Genes

Assume a microarray experiment with i=1, 2, . . . , N genes, whose intensities are measured for k=1, 2, . . . , K breast cancer patients. The log-transformed intensities of gene i and patient k are denoted as y_{i,k}. Log-transformation serves for data “Gaussianization” and variance stabilization purposes, although other approaches, such as the log-linear hybrid transformation of Holder et al. (2001), the generalized logarithm transform of Durbin et al. (2002) and the data-driven Haar-Fisz transform of Motakis et al. (2006), have also been considered in the literature.

Associated with each patient k are a disease free survival time tₖ (in this work DFS time), a nominal clinical event eₖ taking values 0 in the absence of an event until DFS time tₖ or 1 in the presence of the event at DFS time tₖ (DFS event) and a discrete gradual characteristic (histologic grade). Note that in this particular work the events correspond to the presence or absence of tumor metastasis for each of the k patients. Other types of events and/or survival times are possible to be analyzed by the model we will discuss below.

Additional information, which is not utilized in this work, includes patients' age (continuous variable ranging from 28 to 93 years old), tumor size (in millimeters), breast cancer subtype (Basal, ERBB2, Luminal A, Luminal B, No subtype, normal-like), patients' ER status (ER+ and ER−) and distant metastasis (a binary variable indicating the presence or absence of distant metastasis).

Assuming, without loss of generality, that the K clinical outcomes are negatively correlated with the vector of expression signal intensity yᵢ of gene i, patient k can be assigned to the high-risk or the low-risk group according to:

$${x}_{k}^{i} = \{ \begin{matrix}1 & ( high - risk ) , if {y}_{i , k} > {c}^{i} \\ 0 & ( low - risk ) , if {y}_{i , k} \leq {c}^{i}\end{matrix} \qquad ( 3 )$$

where c^{j} denotes the predefined cut-off of the ith gene's intensity level. In the case of positive correlation between the K clinical outcomes and y₁, patient k is simply assigned to one of the two groups according to:

$${x}_{k}^{i} = \{ \begin{matrix}1 & ( high - risk ) , if {y}_{i , k} \leq {c}^{i} \\ 0 & ( low - risk ) , if {y}_{i , k} > {c}^{i}\end{matrix}$$

After specifying xₖⁱ, the DFS times and events are subsequently fitted to the patients' groups by the Cox proportional hazard regression model (Cox, 1972):

log hₖ^{t}(t_{K}|xₖⁱ,βᵢ)=αₗ(tₖ)+βᵢxₖ^{l} (4)

where, as before, hⁱₖ is the hazard function and αᵢ(tₖ)=log hⁱ₀(tₖ) represents the unspecified log-baseline hazard function for gene i; PA is the ith element of the vector β̅ of the model regression parameters to be estimated; and tₖ is the patients' survival time. To assess the ability of each gene to discriminate the patients into two distinct genetic classes, the Wald statistic (W) (Cox and Oakes, 1984) of the βₜ coefficient of model (4) is estimated by minimizing the univariate Cox partial likelihood function for each gene i:

$$L ( {β}_{i} ) = {\prod }_{k = 1}^{K} {\{ \frac{exp ( {β }_{i}^{T} {x}_{k}^{i} )}{{\sum }_{j ∈ R ( {t}_{k} )} exp ( {β }_{i}^{T} {x}_{j}^{i} )} \}}^{{e}_{k}} \qquad ( 5 )$$

where R(tₖ)={j:tⱼ>tₖ} is the risk set at time tₖ and eₖ is the clinical event at time tₖ. The actual fitting of model (4) is conducted by the survival package in R (cran.r-project.org/web/packages/survival/index.html). The genes with the largest βᵢ Wald statistics (Wᵢ's) or the lowest βᵢ Wald P values are assumed to have better group discrimination ability and thus called highly survival significant genes. These genes are selected for further confirmatory analysis or for inclusion in a prospective gene signature set. Note that given βᵢ, one derives the Wald statistic, W, as:

$$var ( {β}_{i} ) = \frac{1}{I ( MLE )}$$

where

$$W = \frac{{β}_{i}^{2}}{var ( {β}_{i} )}$$

and I denotes the Fisher information matrix of the βᵢ parameter. Estimating the Wald P value, simply requires evaluation of the probability:

$$p - value = Pr ( \frac{{β}_{i}^{2}}{var ( {β}_{i} )} > {χ}_{v}^{2} ) \qquad ( 5 )$$

where χᵥ^{z} denotes the chi-square distribution with v degrees of freedom. Typically, v is the number of parameters of the Cox proportional hazards model and in our case v=1. Expression (5) can be derived from the proper statistical tables of the chi-square distribution.

From Eqn. (3) notice that the selection of prognostic significant genes relies on the predefined cut-off value cⁱ that separates the low-risk from the high-risk patients. The simplest cut-off basis is the mean of the individual gene expression values within samples (Kuznetsov, 2006), although other choices (e.g. median, trimmed mean, etc) could be also applied. Two problems, associated with such cut-offs, are: 1) they are suboptimal cut-off values that often provide low classification accuracy or even miss existing groups; 2) the search for prognostic significance is carried out for each gene independently, thus ignoring the significance and the impact of genes' co-expression on the patient' survival.

### SUMMARY OF THE INVENTION

In a first aspect, the present invention proposes in general terms that a cut-off expression value should be selected so as to maximise the separation of the respective survival curves of the two groups of patients. From another point of view, this means that the cut-off expression value is selected such that the partition of subjects which it implies is of maximal statistical significance. This overcomes a possible problem in the known method described above: that if the cut-off expression values are not well-chosen they may provide low classification accuracy even for genes which are very statistically significant for certain ranges of expression value.

A specific expression of the first aspect of the invention is a computerized method for optimising, for each gene i of a set of N genes, a corresponding cut-off expression value cⁱ.

for partitioning subjects according to the expression level of the corresponding gene,
the method employing medical data which, for each subject k of a set of K* subjects suffering from the medical condition, indicates (i) the survival time of subject k, and (ii) for each gene i, a corresponding gene expression value y_{i,k} of subject k;
the method comprising, for each gene i,
(i) for each of a plurality of a trial values of cⁱ:
   (a) identifying a subset of the K* subjects such that y_{i,k} is above the trial value of cⁱ;
   (b) computationally fitting the corresponding survival times of the subjects to the Cox proportional hazard regression model, said fitting using, for subjects within the subset, a regression parameter βᵢ corresponding to the gene i; and
   (c) obtaining from the regression parameter βᵢ, a significance value indicative of prognostic significance of the gene;
(ii) identifying the trial cut-off expression value for which the corresponding significance value indicates the highest prognostic significance for the gene i.

The cut-off expression value y_{i,k} here may be a logarithm of the measured expression intensity of gene i in patient k (as in the background section above, and in following description of specific embodiments). However, the expression value y_{i,k} may alternatively be any other transformation of the measured expression insensity, or indeed the raw intensity itself.

In a second aspect, the invention proposes in general terms that pairs of genes are selected. For each gene pair, we generate a plurality of models, each of which represents a way of partitioning a set of subjects based on the expression values of the pair of genes. We then identify those gene pairs for which one of the models has a high prognostic significance. This overcomes a problem of the known method described above that the search for prognostic significance is carried out for each gene independently, thus neglecting any significance of genes' co-expression on patient survival.

A specific expression of the second aspect of the invention is a computerized method for identifying one or more pairs of genes, selected from a set of N genes, which are statistically associated with prognosis of a potentially-fatal medical condition,

the method employing medical data which, for each subject k of a set of K* subjects suffering from the medical condition, indicates (i) the survival time of subject k, and (ii) for each gene i, a corresponding gene expression value y_{i,k} of subject k; the method comprising:
(i) for each of the N genes obtaining a corresponding cut-off expression value;
(ii) forming a plurality of pairs of the identified genes (i, j with i≠j), and for each pair of genes:
   (1) forming a plurality of models m_{i,j}, each model m_{i,j} including a comparison of the corresponding cut-off expression values d and c of the respective levels of expression y_{i,k},y_{j,k} of the genes i,j in the set of K* subjects;
(2) for each model determining, a respective subset of the K* subjects using the model;
(3) computationally fitting the corresponding survival times of the subjects to the Cox proportional hazard regression model, said fitting using, for subjects within each of the subsets, a corresponding regression parameter βᵢⱼ^{m} corresponding to the model m_{i,j}, and
(4) obtaining from the regression parameters βᵢⱼ^{m}, a significance value indicative of prognostic significance of the model; and
(iii) identifying one or more of said pairs of genes i,j for which the corresponding significance values for one of the models have the highest prognostic significance.

The first and second aspects of the invention may be used in combination. That is, cut-off expression values for individual genes derived according to the first aspect of the invention may be employed in a method according to the second aspect of the invention.

Note that the “expression values” referred to in the specific expressions of the invention may be the direct outputs of expression value measurements, but more preferably are the logarithms (e.g. natural logarithms) of such measurements, and optionally may have been subject to a normalisation operation.

In either the first or second aspect of the invention, the medical data preferably includes nominal data which indicates for each patient, whether one or more clinical events have occurred. For example, the nominal data may indicate whether tumour metastasis had occurred by the survival time. The significance value may be calculated using a formula which incorporates the nominal data. Alternatively or additionally, it can be used to select a subset of the patients, such that the clinical data for that subset of patients is used in the method.

In either the first or second aspect of the invention, the survival time may be an actual survival time (i.e. a time taken to die) or a time spent in a certain state associated with the medical condition, e.g. a time until metastasis of a cancer occurs.

Furthermore, in either the first or second aspect of the invention the K* subjects may be a subset of a larger dataset of K (K>K*) subjects. For example, the data for K* subjects can be used as training data, and the rest used for validation.

Alternatively, a plurality of subsets of the K subjects can be defined, and the method defined above is carried out independently for each of the subsets. Each of these subsets of the K subjects is a respective “cohort” of the subjects; if the cohorts do not overlap, they are independent training datasets. Note that each time the method is performed for a certain cohort, K* denotes the number of subjects in that cohort, which may be different from the number of subjects in other of the cohorts. After this, there is a step of discovering which pairs of genes were found to be significant for all the cohorts.

Once one or more genes, or pairs of significant genes, are identified by a method according to either the first or second aspect of the invention, they can be used to obtain useful information in relation to a certain subject (typically not one of the cohort(s) of subjects) using a statistical model which takes as an input the ratio(s) of the expression values of the corresponding identified pair(s) of genes. The information may for example be susceptibility to the medical condition, the or prognosis (e.g. relating to recurrence or death) of a subject suffering from the condition.

The invention may be expressed, as above, in terms of a method implemented using a computer, or alternatively as a computer system programmed to implement the method, or alternatively as a computer program product (e.g. embodied in a tangible storage medium) including program instructions which are operable by the computer to perform the method. The computer system may in principle be any general computer, such as a personal computer, although in practice it is more likely typically to be a workstation or a mainframe supercomputer.

In a third aspect, the present invention proposes a kit, such as a microarray, for detecting the expression level of a set of genes, the set having no more than 1000 member, or no more than 100 members, or no more than 20 members, and including
(a) at least one of BRRN1; FLJ11029; C6orf173; STK6; MELK; and/or
(b) at least one of the pairs: (i) SPAG5-ERCC6L, (ii) CENPE-CCNE2, (iii) CDCA8-CLDN5, (iv) CCNA2-PTPRT, (v) Megalin (LRP2) and integrin alpha 7 (ITGA7), (vi) NUDT1 and NMU genes, and (vii) HN1 and CACNA1D.

### BRIEF DESCRIPTION OF THE FIGURES

FIG. 1 shows a histogram of Disease Free Survival (DFS) times of (A) Stockholm and (B) Uppsala cohort for subjects with tumour metastasis (Event=1) and without tumour metastasis (Event=0). The dotted lines indicate the DFS threshold values.

FIG. 2 is a flow diagram of a first method according to the invention.

FIG. 3 shows a plot of log p-values against cut-off expression value levels for the LRP2 and ITGA7 prognostic genes in (a) Stockholm cohort and (b) Uppsala cohort. The dashed line indicates the log (p-value) corresponding to the Data driven grouping (DDg) cut-off expression value obtained by the method of FIG. 2. This cut-off expression value is marked by a cross on the dashed line. The dotted line indicates the log (p-value) corresponding to the Mean Based grouping (MBg) value, which is marked by a cross on the dotted line.

FIG. 4 illustrates the four possible ways in which the expression values of a pair of genes i and j can be realized in relation to respective cut-off expression values d and d.

FIG. 5 is a flow diagram of a second method according to the invention.

FIG. 6, which is composed of FIG. 6A to 6G, shows experimental data comparing a known method, and the methods of FIGS. 1 and 4, using clinical data from the for two gene pairs for the “Stockholm” cohort of patients.

FIG. 7, which is composed of FIG. 7A to 7E, shows experimental data for comparing the known method (FIGS. 7B and 7D) and the method of FIG. 4 (FIGS. 7A and 7C), and numerical data (FIG. 7E), for the same gene pair but using the “Uppsala” cohort of patients.

FIG. 8 shows the Kaplan-Meier plot for survival of the Uppsala patients in groups of grades 1 and 1-like versus grades 3 and 3-like, based on a 5 gene signature.

### DEFINITIONS

“Array” or “microarray,” as used herein, comprises a surface with an array, preferably an ordered array, of putative binding (e.g., by hybridization) sites for a sample which often has undetermined characteristics. An array can provide a medium for matching known and unknown nucleic acid molecules based on base-pairing rules and automating the process of identifying the unknowns. An array experiment can make use of common assay systems such as microplates or standard blotting membranes, and can be worked manually, or make use of robotics to deposit the sample. The array can be a macro-array (containing nucleic acid spots of about 250 microns or larger) or a micro-array (typically containing nucleic acid spots of less than about 250 microns).

The term “cut-off expression value” represented by c (followed with the proper superscript; e.g. cⁱ) refers to a value of the expression level of a particular nucleic acid molecule (or gene) in a subject. The cut-off expression value is used to partition the subjects into classes, according to whether the expression level of the corresponding gene is below or above the cut-off expression value. Note that it makes no difference whether all subjects for which the expression value is actually equal to the cut-off value are classified as being in one class or alternatively in the other.

The term “gene” refers to a nucleic acid molecule that encodes, and/or expresses in a detectable manner, a discrete product, whether RNA or protein. It is appreciated that more than one nucleic acid molecule may be capable of encoding a discrete product.

The term includes alleles and polymorphisms of a gene that encodes the same product or an analog thereof.

There are various methods for detecting gene expression levels. Examples include Reverse-Transcription PCR, RNAse protection, Northern hybridisation, Western hybridisation, Real-Time PCR and microarray analysis. The gene expression level may be determined at the transcript level, or at the protein level, or both. The nucleic acid molecule or probe may be immobilized on a support, for example, on an array or a microarray. The detection may be manual or automated. Standard molecular biology techniques known in the art and not specifically described may be employed as described in Sambrook and Russel, Molecular Cloning: A Laboratory Manual, Cold Springs Harbor Laboratory, New York (2001).

Gene expression may be determined from sample(s) isolated from the subject(s).

### DETAILED DESCRIPTION OF THE EMBODIMENTS

Embodiments of the methods will now be explained with reference to the figures, and experimental results are presented which were generated using two cohorts of subjects: the Uppsala and Stockholm cohorts.

### 1. Subjects and Tumour Specimens Used in Experiments

The clinical characteristics of the subjects and the tumour samples of Uppsala and Stockholm cohorts have been summarised in Ivshina et al., 2006. The Stockholm cohort comprised Kₛ=159 subjects with breast cancer, operated on in Karolinska Hospital from Jan. 1, 1994, through Dec. 31, 1996, and identified in the Stockholm-Gotland breast Cancer registry. The Uppsala cohort involved Ku=251 subjects representing approximately 60% of all breast cancers resections in Uppsala County, Sweden, from Jan. 1, 1987, to Dec. 31, 1989. Information on the subjects' disease free survival (DFS) times/events and the expression patterns of approximately 30000 gene transcripts (representing N=44928 probe sets on Affymetrix U133A and U133b arrays) in 315 primary breast tumours were obtained from NCBI Gene Expression Omnibus (GEO) (Stockholm data set label is GSE4922; Uppsala data set label is GSE1456). The microarray intensities were calibrated (RMA) and the probe set signal intensities were log-transformed and scaled by adjusting the mean signal to a target value of log 500 (Ivshina et al., 2006). In this study, Affymetrix U133A and 133b probesets (232 gene signatures) was used to provide classification of the low- and high-aggressive cancer sub-types described in Ivshina et al., 2006. For each of these patients, there was date specifying the disease free survival time (DFS) of an event (tumor metastasis). and the actual occurrence of this event (a binary variable taking the values 1=occurrence of the event, 0=no occurrence of the event).

### 2 Training Data

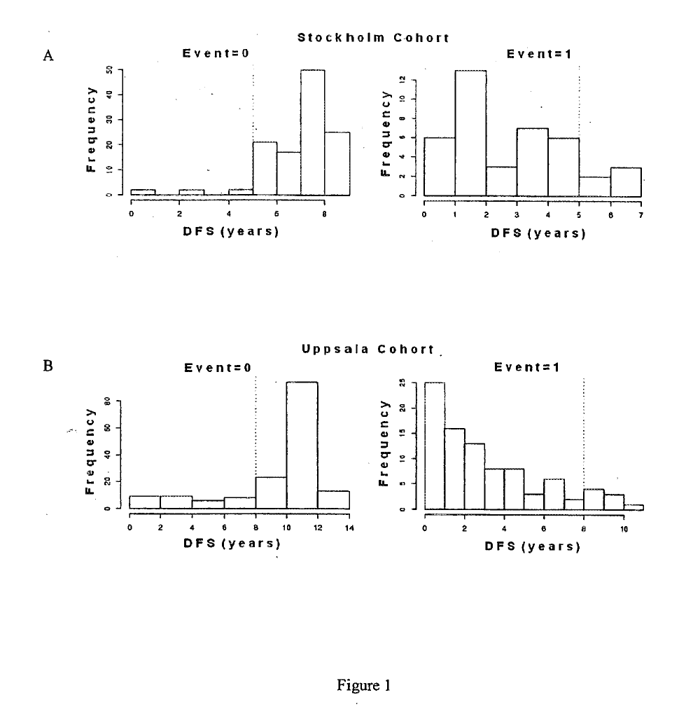

FIG. 1 shows the distribution of DFS survival time (below, we refer to the DFS survival time for the k-th subject as tₖ) for the Stockholm cohort (FIG. 1A) and the Uppasla cohort (FIG. 1B). Each of FIG. 1A and FIG. 1B includes a separate histogram for each of two categories of patients: those for whom tumour metastasis had occurred (this possibility is referred to in FIG. 1 as “Event=1”, and below we refer to this as eₖ=1) and those for whom it had not (this possibility is referred to in FIG. 1 as “Event=0”, and below we refer to this as eₖ=0).

As may be seen in FIG. 1, most of the patients are “typical responders” by which we mean that those for whom Event=1 have short survival times, and those for whom Event=0 have long survival times. In other words we noticed that tumor metastasis typically occurs at a short time (in years) after the beginning of the experiment, whereas the frequency of the occurrence decreases as time increases. To this extent, FIG. 1 shows that our data consists of two different distribution of survival times (one for patients without tumor metastasis and one with patients with metastasis). The data that do not agree with this observation are considered as outliers or data from non-typical patients.

The present embodiments used only the “typical” data as training data. In other words, there was a pre-processing of the data to identify only typical patients: data from subjects who satisfy the above Event and tₖ relationship. Then we apply our methods on the typical (or training) data only (data from responders that satisfy the above Event and tₖ relationship), estimate a cutoff value for each gene by (1) and use these estimates to the whole set of patients to infer about prognostic significance by (2).

Based on visual inspection of FIG. 1, the inventors considered that the part of the Stockholm cohort which should be used as training data is the data from “typical” subjects with tₖ>5 years and eₖ=0, or tₖ<5 years and eₖ=1. The 5 year cut-off is shown by the dashed line in FIG. 1A. This resulted in 148 Stockholm training set subjects. Following the same procedure for the Uppsala data, the threshold was set at 8 years (see the dashed line in FIG. 1B), which resulted in 212 Uppsala training set subjects.

### 3 Nomenclature

The terminology explained in the background section of this document is followed in the following explanation of the embodiments and some comparative examples. In these embodiments and comparative examples, the number of patients in the training set is denoted K* which is less than K, the total number of patients about whom data existed. The subjects of the training set are labelled k=1, . . . , K*. The DFS survival time for the k-th subject is referred to as tₖ, and whether or not tumour metastatis has occurred is referred to as eₖ. Each of the embodiments, and the comparative example, involve fitting a set of survival times to an equation such as Eqn. (4). The fitting to Eqn. (4) was conducted by the survival package which can be found at the following website: cran.r-project.org/web/packages/survival/index.html. See also the references Cox, D. R and Snell, E. J (1968) and Cox, D. R. and Oakes, D (1984). This made it possible to find a Wald statistic for each gene/gene-pair in the matter explained in the background section of this document.

### 4. Finding Cut-Off Values and Identifying Single Genes: A First Embodiment and a Comparative Example

We now discuss estimation of a cut-off expression value for each gene. This is done first using the prior art method discussed above (Mean based grouping—“MBg”) and using a embodiment of the invention illustrated in FIG. 1 (Data driven grouping—“DDg”).

### 4.1 Comparative Example 1

### Mean Based Grouping (MBg)

For each gene i of the training set, the respective mean μᵢ of the values y_{i,k} of the subjects in the training set was found using the K* training set patients. Note that K*<K and that in Stockholm Kₛ=159 and K_{s*}=148 while in Uppsala Kᵤ=251 and K_{u*}=212 (for simplicity we drop the s and u subscripts in the following paragraphs). The subjects of the training set were then grouped according to whether their values of y_{i,k} were above or below a cut-off cⁱ=μᵢ. Equation (3) was used to generate a set of values {xᵢ^{k}}, and from this a set of corresponding values βᵢ was generated by fitting Equation (4). The prognostic significance of gene i was evaluated by reporting the p-value of the estimated βᵢ. The genes with significantly small p-values (p<0.05 or p<0.01) were selected.

Schematically, the steps we follow are:
1. For gene i=1, estimate the mean expression signal μᵢ from the training set of K* patients and set the grouping cutoff cⁱ=μᵢ. Alternatively, one could estimate the median or the trimmed-mean expression signal from the set of K* patients and set accordingly the grouping cutoff equal to these values (these are the median- and trimmed-mean based grouping methods that do not discussed further here because they lead to similar results to the ones of mean-based method).
2. Group the k=1, 2, . . . , K patients according to

$${x}_{k}^{i} = \{ \begin{matrix}1 & ( high - risk ) , if {y}_{i , k} > {c}^{i} \\ 0 & ( low - risk ) , if {y}_{i , k} \leq {c}^{i}\end{matrix} \\ or \\ {x}_{k}^{i} = \{ \begin{matrix}1 & ( high - risk ) , if {y}_{i , k} \leq {c}^{i} \\ 0 & ( low - risk ) , if {y}_{i , k} > {c}^{i}\end{matrix}$$

with cⁱ=μᵢ.
3. Evaluate the prognostic significance of gene i by reporting the P value of the estimated βᵢ from model

log hₖⁱ(tₖ|xₖ^{t},βᵢ)=αᵢ(tₖ)+βᵢxₖ^{t}
4. Iterate for all genes i=2, . . . , N.
5. Select as prognostic significant the genes with significantly small P values (p<alpha=1%).

### 4.2 First Embodiment

### Data-Driven Grouping (DDg)

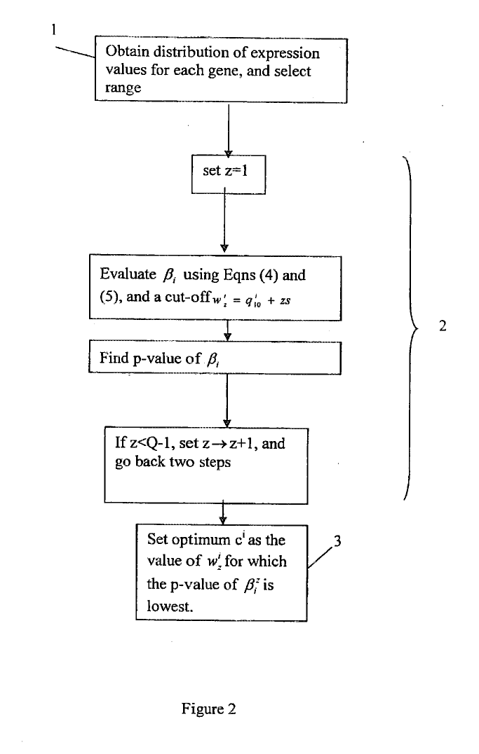

The first embodiment is explained with reference to FIG. 2. For each gene i, the distribution of the K signal intensity values y_{i,k} was computed, and 10^{th} quantile (q₁₀ⁱ) and the 90^{th} quantile (q₉₀ⁱ) were derived (step 1). Within a range (q₁₀ⁱ, q₉₀ⁱ), a search was performed for the value that most successfully discriminates the two unknown genetic classes, which corresponds to the minimum βᵢ^{z} p-value (here z=1, . . . , Q). In step 2 of FIG. 2, the following sub-steps were performed:

1. Form the candidate cut-offs vector of dimension 1×Q, w̅ⁱ=y_{i,k}^{∞}, where y_{i,k}^{∞} is the log-transformed intensities within (q₁₀ⁱ, q₉₀ⁱ) and Q is the number of elements in w¹. Each element of the w̅ⁱ is a trial cut-off value. For i=1 and cⁱ= w̅ₖⁱ, z=1, . . . , Q use

$${x}_{k}^{i} = \{ \begin{matrix}1 & ( high - risk ) if {y}_{i , k} > {c}^{i} \\ 0 & ( low - risk ) if {y}_{i , k} \leq {c}^{i}\end{matrix} \\ or \\ {x}_{k}^{i} = \{ \begin{matrix}1 & ( high - risk ) if {y}_{i , k} \leq {c}^{i} \\ 0 & ( low - risk ) if {y}_{i , k} > {c}^{i}\end{matrix}$$

to separate the K* subjects with cⁱ.

For z=1 (the first element in w̅ⁱ) evaluate the prognostic significance of gene i given cut-off w̅ₓ¹ by estimating the βᵢ² from log hₖⁱ (tₖ|xₖⁱ,βᵢ^{c})=αᵢ(tₖ)+βᵢ²xₖ^{l} and

$$L ( {β}_{i}^{z} ) = {\prod }_{k = 1}^{K} {\{ \frac{exp ( {β }_{i}^{z} {x}_{k}^{i} )}{{\sum }_{j ∈ R ( {t}_{k} )} exp ( {β }_{i}^{z} {x}_{j}^{i} )} \}}^{{e}_{k}} . \qquad$$

3. Iterate for z=2, . . . , Q to estimate the Q p-values (corresponding to Q distinct cut-off expression value levels) for each i. The “optimal” cut-off expression value cⁱ for each i is the taken as the one with the minimum βᵢ² p-value, provided that the sample size of each group is sufficiently large (formally above 30) and Cox proportional hazards model is plausible.
1. Using this cut-off, evaluate the prognostic significance of gene i by estimating the βᵢ from Eqn. (4) and Eqn (5) for the full set of patients.
2. Iterate steps 1 to 4 for i=2, . . . , N.

In step 3, the “optimal” cut-off expression value for each i is the taken as the one with the minimum βᵢ^{z} p-value, provided that the sample size of each group is sufficiently large (formally above 30) and model defined by Eqn. (4) is plausible. Note that here βᵢ is substituted by βᵢ^{z} indicating that the search was for the βᵢ that leads to the best cut-off expression value.

In order to validate the significance of the findings (in terms of the estimated p-values), the Stockholm and Uppsala samples were bootstrapped and the 99% confidence intervals for the βᵢ coefficients of Eqn. (4) were estimated. We use the non-parametric residuals bootstrap for the proportional hazards model of Loughin (1995) using the boot package in R. Specifically, the algorithm works as follows sequentially for each gene i:
1. Estimate βᵢ of model:

log hₖ^{t}(t^{k}|xₖ^{t},βₜ)=α₁(tₖ)+(β₁xₖ^{t}
by maximizing the likelihood:

$$L ( {β}_{i} ) = {\prod }_{k = 1}^{K} {\{ \frac{exp ( {β }_{i}^{T} {x}_{k}^{i} )}{{\sum }_{j ∈ R ( {t}_{k} )} exp ( {β }_{i}^{T} {x}_{j}^{i} )} \}}^{{e}_{k}} \qquad$$

2. Calculate the independent and identically (Uniform in [0,1]) distributed generalized residuals, calculated in Cox and Snell (1968) and Loughin (1995) by the “probability scale data”

uₖ=[1−F₀(t)]^{exp(β}^{^{T}}^{x}^{ₖ}^{ⁱ}⁾,k=1, 2, . . . K,
where F₀(t)=P(T≦t|exp(βᵢ^{T}=x¹)=1) denotes the baseline failure time distribution. Typically, \hat{F}₀(t) is a step function with jumps at the observed failure times (estimated automatically in the survival package), which does not affect the Uniformity of the generalized residuals (Loughin, 1995)
3. Consider the pairs {(u₁,e₁), . . . , (u_{K},e_{K})} and resample with replacement B pairs of observations (B bootstrap samples) {(u₁^{(b)},e₁^{(b)}), . . . , (u_{K}^{(b)}, e_{K}^{(b)})}, b=1, . . . . B (a typical bootstrap step)
4. Calculate the probability scale survival times

$${t}_{k}^{( b )} = 1 - {[ {u}_{k}^{( b )} ]}^{1 / exp ( {β}_{i}^{T} {x}_{k}^{i} )}$$

$${t}_{k}^{( b )} = 1 - {[ {u}_{k}^{( b )} ]}^{1 / exp ( {β}_{i}^{T} {x}_{k}^{i} )}$$

and estimate the bootstrap coefficients βᵢ⁽¹⁾, βᵢ⁽²⁾, . . . , βᵢ^{(B)} by numerically maximizing the partial likelihood:

$${L}^{( b )} ( {β}_{i} ) = {\prod }_{k = 1}^{K} {\{ \frac{exp ( {β }_{i}^{T} {x}_{k}^{i} )}{{\sum }_{j ∈ {R}^{( b )} ( {t}_{k} )} exp ( {β }_{i}^{T} {x}_{j}^{i} )} \}}^{{e}_{i}^{( b )}}$$

$$b = 1 , … , B$$

Based on these coefficients, we estimate and report the Bias-Corrected accelerated (BCa) bootstrap confidence intervals for each βᵢ coefficient that correct the simple quantile intervals of βᵢ for bias and skewness in their distribution (Efron and Tibshirani, 1994). A detailed discussion (theory and applications) on BCa intervals is given in Efron and Tibshirani (1994). Here the 99% BCa were estimated by the boot package in R. Bootstrap test p-values based on quantile method gave similar results using the Wald test.

### 4.3 Identification of Genes

The gene expression profiles were correlated with clinical outcome (disease free survival time; DFS) in the two cohorts with the intention of identifying specific genes that predict survival. It was found that a large fraction of the genes had some correlation with survival, and could be used to predict survival.

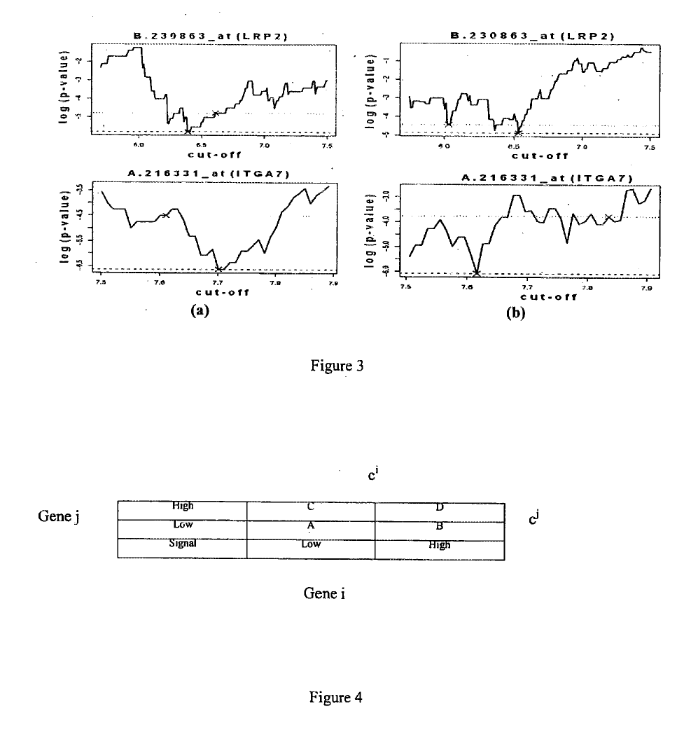

FIG. 3 shows the results of the comparative example and first embodiment for the LRP2 and ITGA7 prognostic genes in (a) Stockholm cohort and (b) Uppsala cohort. In each of the four graphs, the dashed line indicates the log (p-value) corresponding to the Data driven grouping (DDg) cut-off expression value obtained by the method of FIG. 2.

This cut-off expression value is marked by a cross on the dashed line. The dotted line in each of the graphs indicates the log (p-value) corresponding to a MBg cut-off found by method of section 4.1, and this is cut-off is marked by a cross on the dotted line. FIG. 3 suggests that the data-driven grouping improves prediction of subjects' survival compared to the mean based approach.

### 4.4 Survival Significance of Genes of Genetic Grade Signature for the Stockholm Cohort

The mean-based grouping and the data-driven grouping with the estimated cut-off expression value were repeated for the full set of 159 subjects of the Stockholm Cohort to estimate the Wald p-values for each of the 264 probe sets (representing 232-gene genetic grade signature (Ivshina et al., 2006). At 1% significance level, MBg identified 151 probesets (148 prognostic gene signatures), while DDg identified 195 probesets (192 prognostic gene signatures). 82 of the 100 top-level survival related probesets of the two approaches are common. For the genes with p-values lower than 0.1%, the methods are highly reproducible. In this case, ˜99.0% of the MBg probesets was also present in the DDg list.

Bias-Corrected accelerated confidence intervals (BCa Cl's) were used to confirm the Wald statistic estimates. By estimating the 99% BCa Cl's, 118 and 145 probesets were predicted by MBg and DDg methods, respectively, as survival significant probesets. As a comparison between Wald statistic and BCa, 52 probesets (Wald-only positives) with significant Wald p-values (at alpha=1%) were not significant by BCa bootstrapping; while 2 genes (Wald-only negatives) for which the p-values were not significant were significant with BCa bootstrapping for the DDg selected group set. For the MBg selected probesets, the 99% BCa Cl's found 118 significant probesets with 38 Wald-only positives and 2 Wald-only negatives.

### 5.1 Identifying Synergetic Gene Pairs: A Second Embodiment

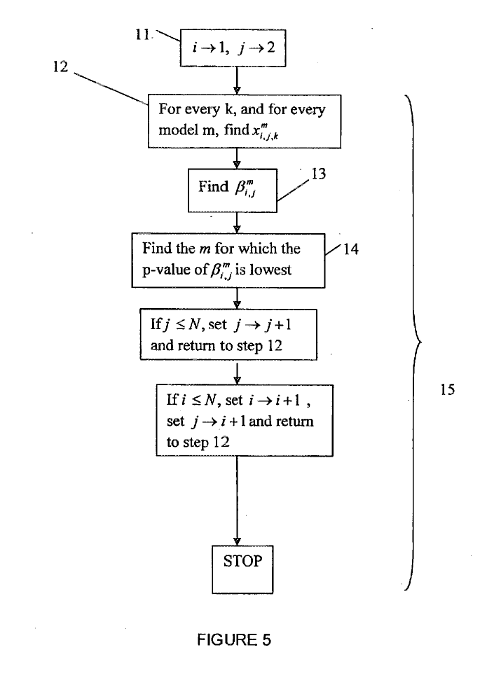

A second embodiment of the invention is explained with reference to FIGS. 4 and 5. The approach resembles the idea of Statistically Weighted Syndromes algorithm (Kuznetsov et al., 2006). For a given gene pair i, j, i≠j, and respective individual cut-off expression values cⁱ and c^{j}, we define seven “models”, each of which is a possible way in which the expression levels of the two genes might be significant. Then we test the data to see if any of the seven models are in fact statistically significant. The models are defined using the concept of FIG. 4, which shows how a 2-D area having y_{i,k}, y_{j,k} as axes. The 2-D area is divided into four regions A, B, C and D, defined as follows:

A: y_{i,k}<cⁱ and y_{j,k}<c^{j}

B: y_{i,k}≧cⁱ and y_{j,k}<c^{j}

C: y_{i,k}<cⁱ and y_{j,k}≧c^{j}

D: y_{i,k}≧cⁱ and y_{j,k}≧c^{j}

Each of the seven models is then defined as a respective selection from among the four regions:

Model 1 is that a subject's prognosis is correlated with whether the subject's patient's expression levels are within regions A or D, rather than B or C.

Model 2 is that a subject's prognosis is correlated with whether the subject's patient's expression levels are within regions A, B or C, rather than D.

Model 3 is that a subject's prognosis is correlated with whether the subject's patient's expression levels are within regions A, C or D, rather than B.

Model 4 is that a subject's prognosis is correlated with whether the subject's patient's expression levels are within regions B, C or D, rather than A.

Model 5 is that a subject's prognosis is correlated with whether the subject's patient's expression levels are within regions A, B or D, rather than C.

Model 6 is that a subject's prognosis is correlated with whether the subject's patient's expression levels are within regions A or C, rather than B or D.

Model 7 is that a subject's prognosis is correlated with whether the subject's patient's expression levels are within regions A or B, rather than C or D.

Note that model 6 is equivalent to asking only whether the subject's expression level of gene 1 is below of above c¹ (i.e. it assumes that the expression value of gene 2 is not important). Model 7 is equivalent to asking only whether the subject's expression for gene 2 is above or below c² (it assumes that the expression value of gene 1 is not important). Thus, models 1-5 are referred to as “synergetic” (1-5), and the models 6 and 7 as “independent”.

The algorithm of the second embodiment evaluates the significance of all possible gene pairs i, j as follows (see FIG. 5):
1. Set i=1 and j=2 (step 11).
2. For each of the 7 models, obtain the respective sub-set of the subjects whose expression values for genes i and j obey the respective set of the conditions (6). For example, for model 1, we obtain the subset of the K* subjects whose expression values obey conditions A, B or C. This is the subset of the subjects for which y_{i,k}<cⁱ and/or y_{j,k}<c^{j} (i.e. the set of subjects which for which condition D is not obeyed). Let us define a parameter x_{i,j,k}^{m}, where x_{i,j,k}^{m}=1 if and only if, for genes i and j, and model m (m=1, . . . 7), the expression levels y_{i,k} and y_{j,k} meet the conditions of model m.
3. Fit the survival values to:

log h^{i,j}ₖ(tₖ|x_{i,j,k}^{m},β_{i,j}^{m})=α_{i,j}(tₖ)+β_{i,j}^{m}·x_{i,j,k}^{m}, (7)
4. Estimate the seven Wald p-values β_{i,j}^{m}. Provided that the respective groups sample sizes are sufficiently large and the assumptions of the Cox regression model are satisfied, the model is the one with the smallest β_{i,j}^{m} p-value.
5. Iterate steps 12 to 14 for all pairs of genes.

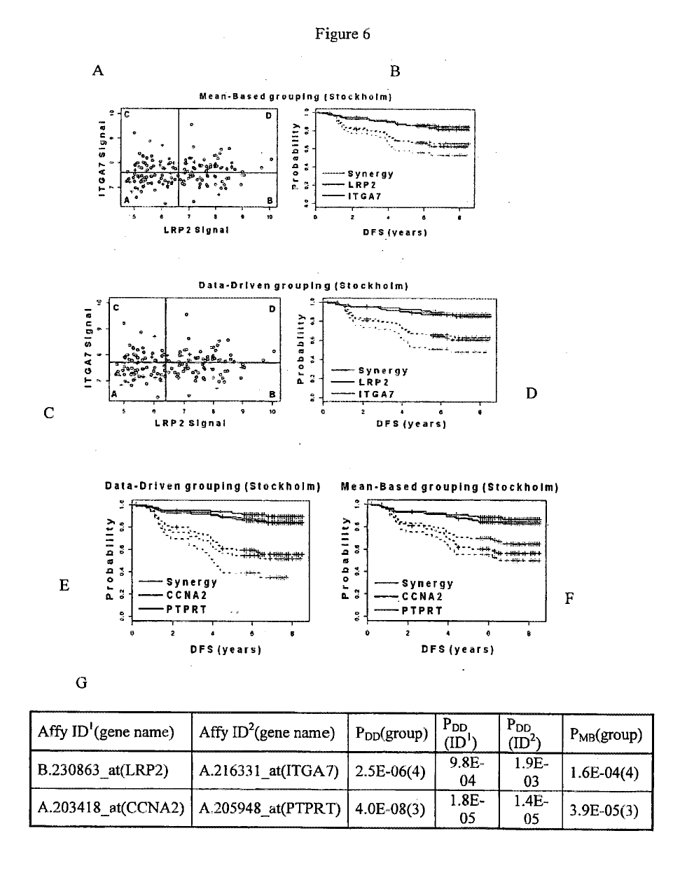

The algorithm above was then applied to the data set in order to examine whether gene pairing could improve the prognostic outcome for certain survival significant genes. All the possible 34716 probeset pairs (0.5×264×263) of the study were considered. FIG. 6 presents the synergetic grouping results for two selected gene pairs: LRP2-ITGA7 and CCNA2-PTPRT. These pairs had been chosen based on two criteria; Criterion 1: their synergy (as indicated by the p-values) was highly significant; Criterion 2: criterion 1 was satisfied in both cohorts.

FIGS. 6A and 6C respectively show clinical data for the LRP2-ITAG7 combination, the crosses representing subjects with DFS time <3 (indicator of “high-risk”) and the circles representing subjects with DFS time >3 (indicator of “low-risk”). The horizontal and vertical lines within the graph indicate the respective cut-off expression values for LRP2 and ITAG7 selected by MBg (FIG. 6A) and DDg (FIG. 6B). Thus, for example, for the LRP2 gene, MMg gives a cut-off expression value of about 6.61 with a p-value of 9.8×10⁻⁴, whereas DDg (the method of FIG. 1) gives a cut-off expression value of about 6.38. The ITGA7 cut-off expression value was 7.7 with a p-value of 1.9×10⁻³ (7.6 in MBg). Similarly for the other pair, the CCNA2 DDg cut-off expression value was 5.95 with a p-value of 1.8×10⁻⁵ (6.04 in MBg) and the PTPRT cut-off expression value was 7.25 with a p-value of 1.4×10⁻⁵ (7.52 in MBg).

As shown in the table of FIG. 6G, the method of FIG. 5, when practiced for the gene pair LRP2-ITAG7 using the cut-off expression values for LRP2 and ITGA7 obtained by MBg, selected model 4, and then produced a p-value for the synergy of 1.6×10⁴. By contrast, when the method of FIG. 4 was practiced with the optimized cut-off expression values for LRP2 and ITGA7 from the method of FIG. 1 (e.g. for gene LRP2, the cut-off expression value of 6.38), the method still selected model 4, but the consequent p-value was 2.5×10⁴. Whichever of the sets of cut-off expression values is used, the subjects in A region are identified as “low-risk” subjects, whereas the B, C and D subjects (“high-risk” subjects) while for the CCNA2-PTPRT case the A, C and D (“high-risk”) subjects were separated from the B subjects (“low-risk”).

For the LRP2-ITGA7 case, the subjects at A region (identified as “low-risk” subjects) were separated from the B, C and D subjects (“high-risk” subjects) while for the CCNA2-PTPRT case the A, C and D (“high-risk”) subjects were separated from the B subjects (“low-risk”).

FIGS. 6B and 6D are corresponding Kaplan-Meier curves (Kaplan and Meier, 1958). The solid lines correspond to “low-risk” subjects and the dotted lines to “high-risk” subjects. FIG. 6B shows the two Kaplan-Meier curves for each of (i) for LRP2 using the cut-offs from MBg, (ii) for ITGA7 using the cut-offs from MBg, and (iii) for model 4 using Eqn. (7) and the two cut-off values from MBg . . . 6D shows the two Kaplan-Meier curves for each of (i) for LRP2 using the cut-offs from DDg, (ii) for ITGA7 using the cut-offs from DDg, and (iii) for model 4 using Eqn. (7) and the two cut-off values from DDg.

For the gene pair CCNA2-PTPRT, the corresponding results for performing the method of FIG. 4 using cut-off expression values from MBg was 3.9×10⁻⁵, and using the optimized cut-off expression values from the method of FIG. 1 (i.e. DDg) was 4.0×10⁴. In both cases, model 3 was selected as the most significant. It is evident that using the optimized cut-off expression values gives much greater statistical significance. The large difference in the estimated DDg and MBg p-values was due to the different cut-off expression values estimated. The Kaplan-Meier curves are shown in FIG. 6E (using the cut-off expression values from DDg) and 6F (using the cut-off expression values from MBg). As in FIGS. 6B and 6D, each of these curves shows separately the two curves for each of the two individual genes, and the two curves derived from Eqn. (7) with m=3 which benefit from the synergy of the genes.

For both pairs of genes, the DDg method separates the “low-risk” from the “high-risk” subjects more accurately as shown by the respective Kaplan-Meier curves.

### 6. Comparison of the Results in Sections 4 and 5 Above with Those Obtained Using the Uppsala Cohort

As mentioned above, in the case of the Uppsala cohort, the training set of “typical” patients consisted of Kᵤ*=212 subjects. The the “mean-based” and “data-driven” cut-off expression values for each of the N=264 probesets were derived according to the comparative example and the first embodiment. The the Wald p-values were derived by Eqns. (4) and (5). At 1% significance, DDg found 195 probesets (191 prognostic gene signatures) and MBg found 131 probesets (127 prognostic gene signatures). There was almost perfect reproducibility for the top level MBg probesets (probesets with p-values lower than 0.1%) in the DDg list, while 82% of the top 100 MBg-DDG probesets were common. The 99% BCa Cl's showed results similar to the Stockholm results. For DDg, 168 significant genes (157 by MBg) were found with 28 Wald-only positives (26 by MBg) and 2 Wald-only negatives (1 by MBg). Across the two cohorts, the Wald statistic discovered 165 common probe sets (˜85% of the DDg significant set) while the bootstrap method found 123 common probe sets (˜73% of the bootstrap DDg significant set).

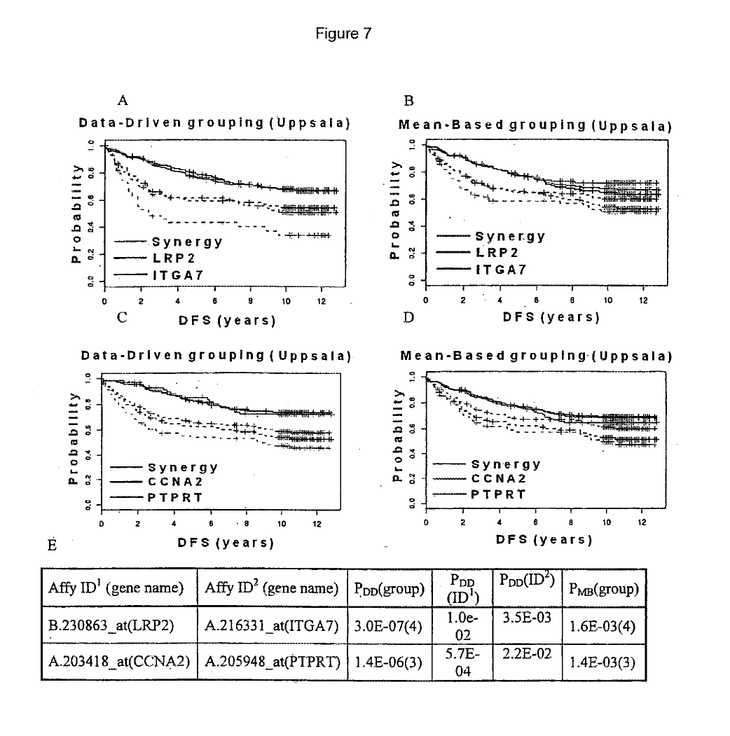

FIG. 7 presents the individual and synergetic prognostic results for the LRP2-ITGA7 and CCNA2-PTPRT genes pairs. The significance of FIGS. 7A to 7E corresponds to that of FIGS. 6D, 6B, 6E, 6F and 6G respectively. Gene pairing with DDg was clearly the best choice in our analysis, since the two “synergy” curves of FIGS. 7A and 7C are by far the best separated. High reproducibility on the findings of the two cohorts, not only for these particular pairs but also for the whole set of important gene pairs identified in Stockholm and Uppsala was observed.

Recently, we discovered a 5-gene signature (6 probesets) which re-classifies tumours with histologic grade 2 into two sub-types, 1-like grade and 3-like grade tumours (Ivshina et al., 2006) that show similar genetic features with grade 1 and grade 3 breast cancer tumours, respectively. Here, we find that all 6 probesets of this 5-gene signature are survival-significant genes identified by the DDg method (BRRN1 (212949_at, p=1.4E-03); FLJ11029 (228273_at, p=1.7E-04); C6orf173 (226936_at, p=6.2E-04); STK6 (208079_s_at, p=3.4E-04; 204092_s_at, p=6.4E-04), MELK (204825_at, p=1.2E-05)), while combinations of these genes with other genes produce synergetic survival effects, suggesting that these genes are representative and robust members of quite a diverse gene regulatory network.

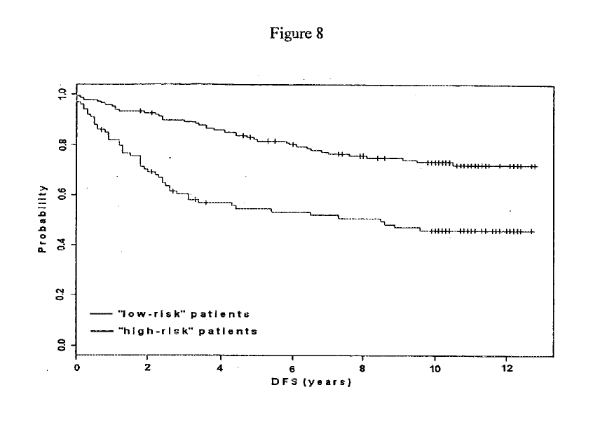

FIG. 8 shows the capability of the 5-genes signature of Ivshina et al., 2006 for predicting subjects' survival outcome for the Uppsala cohort. This figure shows that the survival curves for subjects of joined grade 1 and grade 1-like group are significantly different from the survival curve of subjects of joined grade 3 and grade 3-like group. Using this 5-gene signature we applied the SWS classification method Kuznetsov et al., 2006 and found that the two groups could be discriminated with <7% errors, which suggests that the grouped tumours are different biological entities. Indeed, we observed that the tumours in grade 1&1-like group exhibit mostly “normal-like” and “luminal-A” sub-types, while tumours in 3&3-like group exhibit “luminal-B”, “ERBB2+” and “basal” subtypes. For Stockholm cohort the both SWS signature and clinical sub-typing provide similar classification. In FIG. 8, the survival curve of group of grades 1 & 1-like subjects contains a fraction of subjects with DFS of less than 5 years. This observation suggests the existence of a distinct subgroup of subjects with relatively poor clinical outcome. Using DDg independently and in combination with standard unsupervised techniques (e.g., hierarchical clustering) for grade 1 &1-like group we discovered a set of genes for which expression values could be associated with poor prognosis.

### 7. Functional Significance and Reproducibility of the Genes Associated with Patient Survival

The search for common genes across two independent studies may lead to the identification of the most reliable genes for further analysis. Accordingly, the functional significance and functional reproducibility of the results in the two cohorts were investigated further.

In order to compare the specificity of DDg and MBg in terms of the gene functions they identify, GO (gene ontology) analyses of the top 100 genes of each method were conducted in Panther (Protein Analysis through Evolutionary Relationships) software from the website (pantherdb.org). This grouped the genes into pathways and/or biological processes. Significant enrichment in p53 (DDg p-value=2.4E-03; MBg p-value=2.4E-03) and ubiquitin proteasome (DDg p-value=5.1E-02; MBg p-value=5.2E-02) pathways were identified. Further, significant biological processes include: cell cycle (DDg p-value=1.2E-23; MBg p-value=3.4E-23) and mitosis (DDg p-value=2.4E-11; MBg p-value=6.0E-11). Significant molecular functions include: Microtubule family Cytoskeletal protein (DDg p-value=9.9E-10; MBg p-value=3.0E-10) and protein kinase (DDg p-value−1.5E-04; MBg p-value=6.6E-05).

Similar GO analysis results were observed for the Uppsala data and the findings were well supported by the results of previous studies (Ivshina et al., 2006 and Pawitan et al., 2005). Importantly, the GO analysis produced results very similar to those of the top 1000 survival highly significant genes identified by DDg method from the entire list of 44928 probesets for both the Stockholm and Uppsala cohorts. Table 1 shows the GO analysis of the best gene synergy results according to Criteria 1 and 2. 100 highly significant synergetic pairs represent 44 unique DDg genes and 49 unique MBg genes were identified

TABLE 1
GO analysis of top reproducible 100 gene pairs in Stockholm
and Uppsala cohorts with DDg and MBg methods.

| | DDg | | | MBg | | |
|---|---|---|---|---|---|---|
| T | P | E | O | P | E | O |

Pathways

| | | | | | | | |
|---|---|---|---|---|---|---|---|
| Cell cycle | 29 | 7.94E−06 | 0.12 | 4 | 9.05E−05 | 0.08 | 3 |
| p53 pathway | 136 | 2.66E−05 | 0.57 | 6 | 5.18E−05 | 0.4 | 5 |
| p53 pathway feedback loops 2 | 66 | 1.89E−04 | 0.28 | 4 | 9.87E−04 | 0.19 | 3 |
| DNA replication | 25 | 5.12E−03 | 0.11 | 2 | 2.49E−03 | 0.07 | 2 |
| Folate biosynthesis | 7 | 2.90E−02 | 0.03 | 1 | 2.02E−02 | 0.02 | 1 |
| Formyltetrahydroformate | 10 | 4.12E−02 | 0.04 | 1 | 2.87E−02 | 0.03 | 1 |
| biosynthesis | | | | | | | |
| Ubiquitin proteasome pathway | 89 | 5.00E−02 | 0.37 | 2 | 2.80E−02 | 0.26 | 2 |
| Parkinson disease | 106 | 7.40E−02 | 0.45 | 2 | 3.85E−02 | 0.31 | 2 |

Biological Process

| | | | | | | | |
|---|---|---|---|---|---|---|---|
| Cell cycle | 1009 | 2.56E−28 | 4.25 | 40 | 7.58E−23 | 2.94 | 30 |
| Mitosis | 382 | 4.92E−14 | 1.61 | 18 | 3.54E−13 | 1.11 | 15 |
| Cell cycle control | 418 | 3.13E−10 | 1.76 | 15 | 3.69E−06 | 1.22 | 9 |
| Chromosome segregation | 121 | 2.95E−09 | 0.51 | 9 | 2.99E−09 | 0.35 | 8 |
| Cell proliferation and | 1028 | 3.04E−07 | 4.33 | 18 | 1.43E−06 | 2.99 | 14 |
| differentiation | | | | | | | |
| DNA metabolism | 360 | 4.07E−07 | 1.51 | 11 | 1.02E−07 | 1.05 | 10 |
| DNA replication | 155 | 4.78E−06 | 0.65 | 7 | 6.66E−06 | 0.45 | 6 |
| Protein phosphorylation | 660 | 1.13E−04 | 2.78 | 11 | 6.76E−04 | 1.92 | 8 |
| Meiosis | 84 | 4.68E−04 | 0.35 | 4 | 1.96E−03 | 0.24 | 3 |
| DNA repair | 169 | 7.86E−04 | 0.71 | 5 | 1.55E−03 | 0.49 | 4 |
| Protein modification | 1157 | 1.18E−03 | 4.87 | 13 | 1.89E−03 | 3.37 | 10 |
| Cytokinesis | 115 | 1.49E−03 | 0.48 | 4 | 4.72E−03 | 0.33 | 3 |
| Embryogenesis | 141 | 3.10E−03 | 0.59 | 4 | 7.98E−04 | 0.41 | 4 |
| Developmental processes | 2152 | 3.76E−03 | 9.05 | 18 | 8.82E−03 | 6.26 | 13 |
| Oncogenesis | 472 | 3.94E−03 | 1.99 | 7 | 4.91E−02 | 1.37 | 4 |
| Cell structure | 687 | 8.69E−03 | 2.89 | 8 | 5.01E−02 | 2 | 5 |
| DNA recombination | 44 | 1.51E−02 | 0.19 | 2 | 3.06E−04 | 0.13 | 3 |
| Protein targeting and | 253 | 2.25E−02 | 1.06 | 4 | 6.49E−03 | 0.74 | 4 |
| localization | | | | | | | |
| Cell structure and motility | 1148 | 2.31E−02 | 4.83 | 10 | 1.17E−01 | 3.34 | 6 |
| Mesoderm development | 551 | 2.94E−02 | 2.32 | 6 | 7.71E−02 | 1.6 | 4 |
| Other cell cycle process | 9 | 3.72E−02 | 0.04 | 1 | 2.59E−02 | 0.03 | 1 |
| Lipid, fatty acid and steroid | 770 | 3.73E−02 | 3.24 | 0 | 1.03E−01 | 2.24 | 0 |
| metabolism | | | | | | | |
| Nucleoside, nucleotide and | 3343 | 3.81E−02 | 14.07 | 21 | 2.94E−02 | 9.73 | 16 |
| nucleic acid metabolism | | | | | | | |

Molecular function

| | | | | | | | |
|---|---|---|---|---|---|---|---|
| Microtubule binding motor | 68 | 5.19E−13 | 0.29 | 10 | 3.37E−11 | 0.2 | 8 |
| protein | | | | | | | |
| Microtubule family cytoskeletal | 235 | 4.27E−10 | 0.99 | 12 | 1.90E−09 | 0.68 | 10 |
| protein | | | | | | | |
| Kinase activator | 62 | 7.45E−06 | 0.26 | 5 | 3.55E−05 | 0.18 | 4 |
| DNA helicase | 76 | 1.97E−05 | 0.32 | 5 | 1.48E−03 | 0.22 | 3 |
| Non-receptor serine/threonine | 303 | 4.65E−05 | 1.27 | 8 | 1.96E−03 | 0.88 | 5 |
| protein kinase | | | | | | | |
| Cytoskeletal protein | 878 | 8.62E−05 | 3.69 | 13 | 2.29E−04 | 2.55 | 10 |
| Kinase modulator | 175 | 1.06E−04 | 0.74 | 6 | 1.76E−03 | 0.51 | 4 |
| Protein kinase | 529 | 4.18E−04 | 2.23 | 9 | 8.97E−04 | 1.54 | 7 |
| Helicase | 173 | 8.72E−04 | 0.73 | 5 | 1.43E−02 | 0.5 | 3 |
| Exodeoxyribonuclease | 15 | 1.89E−03 | 0.06 | 2 | 9.13E−04 | 0.04 | 2 |
| Kinase | 684 | 2.47E−03 | 2.88 | 9 | 3.80E−03 | 1.99 | 7 |
| Nucleic acid binding | 2850 | 7.47E−03 | 11.99 | 21 | 1.62E−02 | 8.29 | 15 |
| DNA topoisomerase | 6 | 2.49E−02 | 0.03 | 1 | 1.73E−02 | 0.02 | 1 |
| DNA strand-pairing protein | 6 | 2.49E−02 | 0.03 | 1 | 1.73E−02 | 0.02 | 1 |
| Select regulatory molecule | 1190 | 2.87E−02 | 5.01 | 10 | 5.79E−02 | 3.46 | 7 |
| Non-motor microtubule binding | 74 | 3.93E−02 | 0.31 | 2 | 1.99E−02 | 0.22 | 2 |
| protein | | | | | | | |
| Nuclease | 189 | 4.61E−02 | 0.8 | 3 | 1.80E−02 | 0.55 | 3 |

T = Total number of genes with given GO annotation, P = p-value for significance of GO term enrichment, E = Expected number of genes with given GO annotation, O = Observed number of genes with given GO annotation.

### 8. A Large Number of Gene Pairs can Exhibit a Significantly High Synergetic Survival Effect

In order to compare specificity and sensitivity of the methods in the 2-D case (i.e. using pairs of genes), 34716 pairs of genetic grade signature pairs were considered and the numbers of pairs which provide a significant p-value by the Wald statistic of survival curves <0.01 were counted. The DDg method was more specific and more sensitive than the MBg method. For example using the Stockholm cohort, MBg identified 11778 significant probeset pairs. In comparison, the DDg method identified 16489 significant pairs (˜1.4 times the MBg method), resulting in 4711 DDg pairs unique to DDg. The large difference in the number of significant genes identified by the two methods shows that the DDg method can find interesting genes not located by the MBg method (or any other grouping method based on a single point estimate of yᵢ expression levels). This feature indicates that the DDg method may be particularly appealing for prognostic gene identification.

Using the more stringent p-value of 0.005, it was found that ˜39% of the gene pairs common to both DDg and MBg were false positives. In addition, at a significance level alpha=1% (equivalent to p-value of 0.01), 40% of the unique DDg gene pairs were false positives. In order to reduce Type I errors due to multiple testing (false positives), Bonferroni correction was applied on the Wald test p-values to base the inference on a more stringent significance level. This method identified 1180 significant DDg gene pairs in the Stockholm cohort and 1465 significant DDg gene pairs in the Uppsala cohort, with 53 common pairs in the two cohorts. The respective values derived using MBg approach were 85 significant gene pairs in the Stockholm cohort and 75 significant gene pairs in the Upsala cohort, with no common gene pairs between the two cohorts. For the individual gene analysis, DDg with Bonferroni correction found 97 and 88 significant genes in the Stockholm and Uppsala cohorts, respectively, with 58 common genes between the two cohorts, while the corresponding numbers for MBg were 35 and 36 significant genes (10 common).

The grouping scheme analysis was repeated for the full data set of 44928 probesets. In the Stockholm cohort, the DDg method identified 7473 prognostic significant genes by the Wald test. With Bonferroni correction, the number was 90 prognostic significant genes. In the Uppasala cohort, the respective numbers were 5545 by the Wald test and 55 after Bonferroni correction. Between the two cohorts, 3152 common prognostic genes were identified by the Wald-based DDg test while the MBg method identified 559 (˜18% of DDg). This further supports that the DDg method is able to identify many statistically significant and biologically meaningful prognostic and predictive genes.

Table 2 presents the top 7 gene pairs in terms of the Criteria 1 and 2. These pairs exhibit high synergetic effect in both cohorts and their synergy produces significantly stronger effect than individual grouping (as indicated by the respective p-values in Table 2).

TABLE 2
Top 7 gene pairs in Stockholm and Uppsala cohorts.

| | | P_{S} | P_{U} | | | | |
|---|---|---|---|---|---|---|---|
| Gene¹ | Gene² | (model) | (model) | P¹_{S} | P¹_{U} | P²_{S} | P²_{U} |
| LRP2** | ITGA7** | 2.5E−06 | 3.1E−07 | 9.8E−04 | 1.0E−02 | 1.9e−03 | 3.5e−03 |
| | | (4) | (4) | | | | |
| CCNA2** | PTPRT** | 4.0E−08 | 1.4E−06 | 1.8E−05 | 5.7E−04 | 1.4E−05 | 2.2e−02 |
| | | (3) | (3) | | | | |
| NUDT1** | NMU* | 1.7E−06 | 1.1E−06 | 1.1e−04 | 3.9e−03 | 9.5E−03 | 1.5E−04 |
| | | (2) | (2) | | | | |
| CCNE2** | CENPE** | 1.2E−06 | 6.2E−06 | 9.2E−05 | 2.1E−03 | 7.0E−05 | 1.1e−03 |
| | | (2) | (2) | | | | |
| CDCA8** | CLDN5** | 1.9E−06 | 7.8E−08 | 5.6E−04 | 1.4E−05 | 1.1e−04 | 1.7e−02 |
| | | (3) | (3) | | | | |
| HN1* | CACNA1D | 7.3E−06 | 8.5E−06 | 5.9E−04 | 3.8E−04 | 2.1e−03 | 4.1e−03 |
| | | (3) | (3) | | | | |
| SPAG5** | FLJ20105** | 2.0E−06 | 4.7E−07 | 6.1E−05 | 5.9E−04 | 9.9E−05 | 3.7e−05 |
| | | (2) | (4) | | | | |

P-values of common synergetic genes (and model) of Stockholm (S) and Uppsala (U) cohorts (columns 3-4); P-values of independent grouping (columns 5-8).
**Breast cancer associated genes;
*cancer associated genes.

Survival significant gene are summarized in the table 3.

TABLE 3
Nineteen Survival Significant Genes. Top 7 gene pairs in Stockholm
and Uppsala cohorts. P-values of common synergetic genes (and model)
of Stockholm (S) and Uppsala (U) cohorts (columns 3-4). 5-gene
genetic grading signature genes. “2D” refers to selecting
pairs of genes, “1D” refers to selecting individual genes.

| Gene | P_{S} (model) | P_{U} (model) | Method |
|---|---|---|---|
| LRP2 | 2.5E−06 (4) | 3.1E−07 (4) | 2D |
| ITGA7 | 2.5E−06 (4) | 3.1E−07 (4) | 2D |
| CCNA2 | 4.0E−08 (3) | 1.4E−06 (3) | 2D |
| PTPRT | 4.0E−08 (3) | 1.4E−06 (3) | 2D |
| CCNE2 | 1.2E−06 (2) | 6.2E−06 (2) | 2D |
| CENPE | 1.2E−06 (2) | 6.2E−06 (2) | 2D |
| CDCA8/Borealin | 1.9E−06 (3) | 7.8E−08 (3) | 2D |
| CLDN5 | 1.9E−06 (3) | 7.8E−08 (3) | 2D |
| SPAG5 | 2.0E−06 (2) | 4.7E−07 (4) | 2D |
| FLJ20105/ERCC6L | 2.0E−06 (2) | 4.7E−07 (4) | 2D |
| NUDT1 | 1.7E−06 (2) | 1.1E−06 (2) | 2D |
| NMU | 1.7E−06 (2) | 1.1E−06 (2) | 2D |
| HN1 | 7.3E−06 (3) | 8.5E−06 (3) | 2D |
| CACNA1D | 7.3E−06 (3) | 8.5E−06 (3) | 2D |
| 5-gene signature | 5.20E−06 | NA | |
| BRRN1 | 1.40E−03 | NA | 1D |
| FLJ11029/228273_at | 1.70E−04 | NA | 1D |
| C6orf173/CENPW | 6.20E−04 | NA | 1D |
| STK6 | 3.40E−04 | NA | 1D |
| MELK | 1.20E−05 | NA | 1D |

### DISCUSSION

In the embodiments, the semi-parametric Cox proportional hazard regression model was used to estimate predictive significance of genes for disease outcome as indicated by patient survival times. For a given gene, the optimal partition (cut-off expression value) of its expression domain was estimated by maximising the separation of survival curves related to the high- and low-risk of disease behaviour, as indicated by the Wald statistic derived from the corresponding univariate Cox partial likelihood function. The top-level genes having the largest Wald statistic were selected for further confirmatory GO-analysis and inclusion into gene signatures.

A similar selection procedure was also developed in order to construct two-genes signatures exhibiting synergetic influence on patient survival. This approach was applied to analyse Affymetrix U133 data sets of two large breast cancer cohorts to identify genes and genes pairs related to genetic breast cancer grade signature (Ivshina et al., 2006). The genes that were most significantly correlated with the disease free survival time of breast cancer patients were selected. These genes could be subsequently used as an input in reconstruction analysis of biological programs/pathways associated with aggressiveness of breast cancer (Ivshina et al., 2006).

All genes of 5-gene genetic grading signature are survival significant. Additionally, they are co-regulated in primary breast cancer samples (data not present) and could functionally related to each other. BRRN1 encodes a member of the barr gene family and a regulatory subunit of the condensin complex. This complex is required for the conversion of interphase chromatin into condensed chromosomes. The protein encoded by this gene is associated with mitotic chromosomes, except during the early phase of chromosome condensation. During interphase, the protein has a distinct punctate nucleolar localization. There is a SSB-specific response of condensin I through PARP-1 and a role for condensin in SSB. repair. The protein encoded by STK6 is a cell cycle-regulated kinase that appears to be involved in microtubule formation and/or stabilization at the spindle pole during chromosome segregation. The encoded protein is found at the centrosome in interphase cells and at the spindle poles in mitosis. This gene may play a role in tumor development and progression. A processed pseudogene of this gene has been found on chromosome 1, and an unprocessed pseudogene has been found on chromosome 10. Multiple transcript variants encoding the same protein have been found for this gene. MELK is known to have a critical role in the proliferation of brain tumors, including their stem cells, and suggest that MELK may be a compelling molecular target for treatment of high-grade brain tumors. 2. Maternal embryonic leucine zipper kinase transcript abundance correlates with malignancy grade in astrocytomas 3. the kinase activity of MELK is likely to affect mammary carcinogenesis through inhibition of the pro-apoptotic function of Bcl-GL 4. analysis of MELK substrate specificity and activity regulation 5. pEg3 is a potential regulator of the G2/M progression and may act antagonistically to the CDC25B phosphatase, pEg3 kinase is able to specifically phosphorylate CDC25B in vitro. One phosphorylation site was identified and corresponded to serine 323.

For our work that available information is important in context of validation of our method of identification of patient survival significant genes. In particular, these 3 genes are included in the genetic grade signature which classifies breast cancer patients according to aggressiveness of the cancer disease (Ivshina et al, 2006) and have also used as important clinical markers of breast cancer and other human cancers. MELK is used now as a target for adjuvant therapy in clinical trials. All these genes are survival significant by our DDg estimates.

The biological importance of the DDg survival genes was also demonstrated by the proliferative pattern of the 5-gene grade signature (FIG. 8).

Thus, these genes itself or in pairs or in larger number groups with other genes represent biologically and clinically important survival significant gene signature. We could claim, that such genes and their combinations could be used as “a positive control” for reliable selection of poorly defined/unknown genes which could be promising as novel and important components of critical biological pathways in cancer cells, and novel markers for prognosis and prediction of cancer patients.

In particular, C6orf173 (226936_at, 1D DDg: p=6.2E-04) which is a member of our 5-gene genetic grading signature (Ivshina et al, 2006) and this gene is also a survival significant by our analysis (Kuznetsov et al, 2006; Motakis et al, 2009). It was reported a a cancer up-regulated gene (CUG2) (Lee et al, 2007; Kim et al, 2009). CUG2 was recently renamed CENPW based on the new findings that it is a component of the centromeric complex playing a crucial role in the assembly of functional kinetochore complex during mitosis (Hod et al, 2008).

FLJ11029 (228273_at, p=1.7E-04) is an unknown gene. We found that with very high expression in many cancer tissues and cell lines, however the functions of this gene are unknown. Due to moderate level of evolution conservation and lost of ORF structures (data not shown), FLJ11029 (228273_at) gene could be considered as a novel long non-protein coding gene. In Uppsala and Stockholm cohorts, CUG2(CENPW) and FLJ11029 are strongly positively correlated to each other (Kendal correlation; p<0.0001) and simultaneously with the expression levels of BRRN1, STK6, and MELK (Kendal correlation p<0.0001). Our results of survival analysis suggest that all 5 genes are survival significant and could form essential functional module associated with mitosis phase of breast cancer cells.

Base on these findings we suggest that CUG2, BRRN1, STK6, MELK and can be involved in same regulatory pathway (or sub-network) which is associated with mitotic chromosomes, chromosome condensation and their segregation.

Thus, we suggest a function of genetic grade 5-gene signature genes, related to their biological attributes to coordination and co-regulation dynamics of mitotic chromosomes; these genes provide a synergetic effect on survival of the breast cancer patients and could be used to identify distinct subtypes of breast cancers.

A large number of synergetic gene pairs that were significantly associated with survival of breast cancer patients had been identified and biologically meaningful information about these survival-significant genes may also be postulated. This DDg approach was able to identify interesting targets that were not picked up by other methods, e.g. MBg approach. In order to further test the effectiveness of the DDg approach on the whole gene population of the study, the two methods were applied to the 44928 probesets and the DDg prognostic genes list was found to be twice as large and all genes identified by MBg were also present in the DDg list.

Most of the top-level survival significant genes were related to the cell cycle, and more specifically to the shortest phase of the cell cycle, mitosis (Table 1, Table 2). FIGS. 6E and 6F show an example of a highly significant partition of breast cancer patients by survival (DFS) based on one of cell cycle gene pairs—CCNA2-PTPRT. CCNA2 (Cyclin-A2 or Cyclin A) is an essential gene for the control of the cell cycle at the G1/S (start) and G2/M (mitosis) transitions and it is accumulated steadily during G2 and is abruptly destroyed at mitosis. The paired partner of CCNA2 is a receptor protein tyrosine phosphatise T gene, PRPRT, which regulates cell growth, proliferation and may be important for the STAT3 signalling pathway and development of some cancers. The present survival analysis suggests that this pair of regulatory genes could be involved with other cell cycle genes (see, for example, Table 2) in the control of breast cancer cell development. CCNA2 has second synergistic gene partner, it is CENPE. CENPE (Centrosome-associated protein E) is a kinesin-like motor protein that accumulates in the G2 phase of the cell cycle. Unlike other centrosome-associated proteins, it is not present during interphase and first appears at the centromere region of chromosomes during prometaphase. CENPE is proposed to be one of the motors responsible for mammalian chromosome movement and/or spindle elongation. CDCA8(synonym Borealin) is a synergestic survival significant partner of CENPE. CDCA8 is a component of a chromosomal passenger complex required for stability of the bipolar mitotic spindle. CDCA8 play regulatory role in SUMO pathway including RanBP2 and SENP3. Mitotic regulator Survivin binds as a monomer to its functional interactor Borealin. Thus the both genes CENPE and CDCA8 could be physically co-localized in vicinity of centromeric and spindle regions of mitotic cells and form a functional module controlling key biological processes of cell mitosis. Our goodness-off split survival (DDg) analysis suggests that the both genes could be essential for survival of breast cancer patients.

Another survival significant gene SPAG5 encodes a protein associated with the mitotic spindle apparatus (www.genecards.org/cgi-bin/carddisp.pl?gene=SPAG5&search=SPAG5). By the literature, SPAG5 encoded protein may be involved in the functional and dynamic regulation of mitotic spindles. FLJ20105 ((ERCC6L); www.genecards.org/cgi-bin/carddisp.pl?gene=ERCC6L&search=ERCC6L) is a partner of synergistic survival significant pair of SPAG5. ERCC6L is DNA helicase that acts as an essential component of the spindle assembly checkpoint. Contributes to the mitotic checkpoint by recruiting MAD2 to kinetochores and monitoring tension on centromeric chromatin. Acts as a tension sensor that associates with catenated DNA which is stretched under tension until it is resolved during anaphase. Thus, the both genes of that pair are co-localized (centromeric region) and involved in the same molecular machinery (mitotic spindle assembly).

Thus, based on our analysis, we claim that gene pairs SPAG5-ERCC6L, CENPE-CCNE2, CDCA8-CLDN5 consist of a novel breast cancer associated synergistic survival significant gene pairs playing a important role in dynamics of chromosomes during cell mitosis. Together with the 5-gene genetic grade signature genes they form an integrative functional module essential for breast cancer prognosis and prediction.

Compared to single survival significant genes, gene pairs showed highly significant p-values of Wald statistic (˜10⁻⁸ vs ˜10^{˜5}). This result implied that the pairing procedure itself should be considered as a unique statistical tool for identification of patients with very poor/good prognosis. Interestingly, some of the gene pairs are not directly related to the cell cycle.

Some of the pairs, for example, megalin (LRP2) and itnergrin alpha 7 (ITGA7) have not been discussed for breast cancer classification or patients' survival in the literature. The megalin gene contributes to the endocytic uptake of 25(OH)D3-DBP and activation of the vitamin D receptor (VDR) pathway. The VDR pathway is normally expressed in mammary gland, where it functions to oppose estrogen-driven proliferation and maintain differentiation. LRP2 can be highly expressed in some breast cancer cells. The above suggest that LRP2 participates in negative growth regulation of mammary epithelial cells. Associations of expression of integrin alpha 7 with breast cancer and breast cancer patient survival have not been reported. Megalin and integrin alpha 7 have also not been reported as molecular partners in anti-cancer regulation.

In the present study, several other gene pairs can be related to strong survival significance and produce an interaction effect influencing the likelihood of survival of breast cancer patients (Results, Table 2). These gene pairs, discovered by the present approach, might be grouped into survival significant interactome sub-networks and be an important source for the discovery of new anti-cancer drugs.

HN1-CACNA1D pair. Hematological and neurological expressed 1 protein is a protein that in humans is encoded by the HN1 gene. CACNA1D is a calcium channel, voltage-dependent, L type, alpha 1D subunit. The roles of these genes in breast cancer have been not studied yet.

Mis-incorporation of oxidized nucleoside triphosphates into DNA/RNA during replication and transcription can cause mutations that may result in carcinogenesis or neurodegeneration. The protein encoded by NUDT1 (synonym MTH1) is an enzyme that hydrolyzes oxidized purine nucleoside triphosphates, such as 8-oxo-dGTP, 8-oxo-dATP, 2-hydroxy-dATP, and 2-hydroxy rATP, to monophosphates, thereby preventing mis-incorporation. The encoded protein is localized mainly in the cytoplasm, with some in the mitochondria, suggesting that it is involved in the sanitization of nucleotide pools both for nuclear and mitochondrial genomes. Several alternatively spliced transcript variants, some of which encode distinct isoforms, have been identified. Additional variants have been observed, but their full-length natures have not been determined. A single-nucleotide polymorphism that results in the production of an additional, longer isoform (p26) has been described. NUDT1 plays an important role in protecting cells against H(2)O(2)-induced apoptosis via a Noxa- and caspase-3/7-mediated signaling pathway. Elevated levels of NUDT1 protein is associated non-small cell lung carcinomas. This gene provides synergistic survival effect together with gene neuromedin U (NMU). NMU may be involved in the HGF-c-Met paracrine loop regulating cell migration, invasiveness and dissemination of pancreatic ductal adenocarcinoma. NMU and its cancer-specific receptors, as well as its target genes, are frequently overexpressed in clinical samples of lung cancer and in cell lines, and that those gene products play indispensable roles in the growth and progression of lung cancer cells. NmU expression is related to Myb and that the NmU/NMU1R axis constitutes a previously unknown growth-promoting autocrine loop in myeloid leukemia cells. NMU plays a role in feeding behavior and catabolic functions via corticotropin-releasing hormone. Amino acid variants in NMU associate with overweight and obesity, suggesting that NMU is involved in energy regulation in humans. Overexpression of neuromedin U is associated with bladder tumor formation, lung metastasis and cancer cachexia. Our results suggest important role of NUDT1 and NMU genes in breast cancer progression and the breast patient's survival. Thus, this pair could be considered as novel prognostic and predictive biomarkers for breast cancer.

Thus, our data-driven approach allows the discovery of novel mechanistically important individual genes and small gene signatures that predict cancer patient survival. In doing so, the method can lead to the identification of new potential targets for anti-cancer drugs and facilitate the development of alternative approaches for cancer treatment.

The methods can be extended up to any number of genes. We can check only the “all possible pairs” of the statistically significant individual genes or generally all possible pairs of genes in our study. The number of genes to be used is purely a matter of the researcher's choice.

Table 4 indicates the SEQ ID NO. and the corresponding gene and GenBank reference in the sequence listing.

Where more than one transcript variant exists, the invention may be practiced with any one of the transcript variants, any of several of the transcript variants or all of the transcript variants.

| SEQ ID NO: | Gene | GenBank Reterence |
|---|---|---|
| 1 | LRP2 | LRP2 |
| 2 | ITGA7 transcript variant 2 | ITGA7 transcript variant 2 |
| 3 | CCNA2 | CCNA2 |
| 4 | PTPRT transcript variant 1 | PTPRT transcript variant 1 |
| 5 | FLJ11029 228273_at | FLJ11029 228273_at |
| 6 | C6orf173/CENPW/CUG2 | C6orf173/CENPW/CUG2 |
| 7 | MELK | MELK |
| 8 | NUDT1/MTH1 transcript | NUDT1/MTH1 transcript |
| | variant 4A | variant 4A |
| 9 | NMU | NMU |
| 10 | CCNE2 | CCNE2 |
| 11 | CENPE | CENPE |
| 12 | CDCA8/Borealin | CDCA8/Borealin |
| 13 | CLDN5 transcript variant 2 | CLDN5 transcript variant 2 |
| 14 | HN1 transcript variant 2 | HN1 transcript variant 2 |
| 15 | HN1 transcript variant 3 | HN1 transcript variant 3 |
| 16 | CACNA1D transcript | CACNA1D transcript |
| | variant 1 | variant 1 |
| 17 | SPAG5 | SPAG5 |
| 18 | FLJ20105/ERCC6L | NM_017669.2 |
| 19 | BBRN1/NCAPH | NM_015341.3 |
| 20 | STK6/AURKA transcript | NM_198433.1 |
| | variant 1 | |

### REFERENCES

Breslow, N. E., “Covanance analysis of censored survival data”, Biometrics, vol. 30, pp 89-99, 1974.
Cox D R: Regression Models and Life Tables (with Discussion). Journal of the Royal Statistical Society Series B 1972, 34: 187-220.
Cox, D. R. and Snell, E. J., “A general definition of residuals (with discussion)”, Journal of the Royal Statistical Society, Series B, vol. 30, pp 248-265, 1968.
Cox R. D. and Oakes, D., Analysis of Survival Data. London: Chapman and Hall, 1984.
Efron, B. and Tibshirani, R. J., An Introduction to the Bootstrap. New York: Chapman and Hall, 1994.
Hori T, Amano M, Suzuki A, Backer C B, Welbum J P, Dong Y, McEwen B F, Shang W H, Suzuki E, Okawa K, Cheeseman I M, Fukagawa T. CCAN makes multiple contacts with centromeric DNA to provide distinct pathways to the outer kinetochore. Cell. 2008 Dec. 12; 135(6):1039-52.PMID: 19070575
Ivshina, A. V., George, J., Senko, O et al, “Genetic reclassification of histologic grade delineates new clinical subtypes of breast cancer”, Cancer Research, vol. 66, pp 10292-10301, 2006.
Kaplan, E. L. and Meier, P., “Nonparametric estimation from incomplete observations”. JASA, vol. 53, 457-48, 1958.
Kim H, Lee M, Lee S, Park B, Koh W, Lee D J, Lim D S, Lee S. Cancer-upregulated gene 2 (CUG2), a new component of centromere complex, is required for kinetochore function. Mol Cells. 2009 June; 27(6):697-701. Epub 2009 Jun. 12.PMID: 19533040
Kuznetsov, V. A., Senko, O. V., Miller, L. D. and Ivshina, A., “Statistically Weighted Voting Analysis of Microarrays for Molecular Pattern Selection and Discovery Cancer Genotypes”, International Journal of Computer Science and Network Security, vol. 6, pp 73-83, 2006.
Lee S, Gang J, Jeon S B, Choo S H, Lee B, Kim Y G, Lee Y S, Jung J, Song S Y, Koh S S. Molecular cloning and functional analysis of a novel oncogene, cancer-upregulated gene 2 (CUG2). Biochem Biophys Res Commun. 2007 Aug. 31; 360(3):633-9. Epub 2007 Jun. 28.PMID: 17610844
Loughin, T. M., “A residual bootstrap for regression parameters in proportional hazards model”, J. of Statistical and Computational Simulations, vol. 52, pp 367-384, 1995.
Motakis, E., Nason, G. P., Fryzlewicz, P. and Rutter, G. A., “Variance stabilization and normalization for one-color microarray data using a data-driven multiscale approach”, Bioinformatics, vol. 22, pp 2547-2553, 2006.
Motakis E, Ivshina A V, Kuznetsov V A. Data-driven approach to predict survival of cancer patients: estimation of microarray genes' prediction significance by Cox proportional hazard regression model. IEEE Eng Med Biol Mag. 2009 July-August; 28(4):58-66.
Millenaar, F. F., Okyere, J., May, S. T., van Zanten, M., Voesenek, L. A. C. J. and Peters, A. J. M., “How to decide?Different methods of calculating gene expression from short oligonucleotide array data will give different results”, BMC Bioinformatics, vol. 7, no. 137, 2006.
Pawitan, Y., Bjohle, J., Amler, L., at al, “Gene expression profiling spares early breast cancer patients from adjuvant therapy: derived and validated in two population-based cohorts”, Breast Cancer Research, vol. 7, pp R953-R964, 2005.
Press, W. H., Flannery, B. P., Teukolsky, S. A and Vetterling, W. T., “Numerical Recipes in C: The Art of Scientific Computing”, Cambridge University Press, 1992.
Sambrook and Russel, Molecular Cloning: A Laboratory Manual, Cold Springs Harbor Laboratory, New York (2001).

1-20. (canceled)

21. A kit for detecting the expression level of a set of genes, the set having no more than 1000 members, and comprising:
(a) at least one of BRRN1; FU11029; C6orf173; STK6; MELKU; and/or
(b) at least one of the pairs: (i) SPAG5-ERCC6L, (ii) CENPE-CCNE2, (iii) CDCA8-CLDN5, (iv) CCNA2-PTPRT, (v) Megalin (LRP2) and itnergrin alpha 7 (ITGA7), (vi) NUDT1 and NMU genes, and (vii) HN1 and CACNA1D.

22-32. (canceled)

33. A kit according to claim 21, wherein the set has no more than 100 members.

34. A kit according to claim 21, wherein the set has no more than 20 members.

35. A kit according to claim 21, wherein the kit is a microarray.


SEQUENCE LISTING (20 sequences)

SEQ ID NO: 1 - DNA, 15735 bp, Homo sapiens
ggtctaaagggctttatgcactgtctggagggtggggactggcgcgggtagaaaacgggatgcctcgggcgtgggggcaggcttttggccactaggagctggcggaggtgcagacctaaaggagcgttcgctagcagaggcgctgccggtgcggtgtgctacgcgcgcccacctcccggggaaggaacggcgaggccggggaccgtcgcggagatggatcgcgggccggcagcagtggcgtgcacgctgctcctggctctcgtcgcctgcctagcgccggccagtggccaagaatgtgacagtgcgcattttcgctgtggaagtgggcattgcatccctgcagactggaggtgtgatgggaccaaagactgttcagatgacgcggatgaaattggctgcgctgttgtgacctgccagcagggctatttcaagtgccagagtgagggacaatgcatccccaactcctgggtgtgtgaccaagatcaagactgtgatgatggctcagatgaacgtcaagattgctcacaaagtacatgctcaagtcatcagataacatgctccaatggtcagtgtatcccaagtgaatacaggtgcgaccacgtcagagactgccccgatggagctgatgagaatgactgccagtacccaacatgtgagcagcttacttgtgacaatggggcctgctataacaccagtcagaagtgtgattggaaagttgattgcagggactcctcagatgaaatcaactgcactgagatatgcttgcacaatgagttttcatgtggcaatggagagtgtatccctcgtgcttatgtctgtgaccatgacaatgattgccaagacggcagtgacgaacatgcttgcaactatccgacctgcggtggttaccagttcacttgccccagtggccgatgcatttatcaaaactgggtttgtgatggagaagatgactgtaaagataatggagatgaagatggatgtgaaagcggtcctcatgatgttcataaatgttccccaagagaatggtcttgcccagagtcgggacgatgcatctccatttataaagtttgtgatgggattttagattgcccaggaagagaagatgaaaacaacactagtaccggaaaatactgtagtatgactctgtgctctgccttgaactgccagtaccagtgccatgagacgccgtatggaggagcgtgtttttgtcccccaggttatatcatcaaccacaatgacagccgtacctgtgttgagtttgatgattgccagatatggggaatttgtgaccagaagtgtgaaagccgacctggccgtcacctgtgccactgtgaagaagggtatatcttggagcgtggacagtattgcaaagctaatgattcctttggcgaggcctccattatcttctccaatggtcgggatttgttaattggtgatattcatggaaggagcttccggatcctagtggagtctcagaatcgtggagtggccgtgggtgtggctttccactatcacctgcaaagagttttttggacagacaccgtgcaaaataaggttttttcagttgacattaatggtttaaatatccaagaggttctcaatgtttctgttgaaaccccagagaacctggctgtggactgggttaataataaaatctatctagtggaaaccaaggtcaaccgcatagatatggtaaatttggatggaagctatcgggttacccttataactgaaaacttggggcatcctagaggaattgccgtggacccaactgttggttatttatttttctcagattgggagagcctttctggggaacctaagctggaaagggcattcatggatggcagcaaccgtaaagacttggtgaaaacaaagctgggatggcctgctggggtaactctggatatgatatcgaagcgtgtttactgggttgactctcggtttgattacattgaaactgtaacttatgatggaattcaaaggaagactgtagttcatggaggctccctcattcctcatccctttggagtaagcttatttgaaggtcaggtgttctttacagattggacaaagatggccgtgctgaaggcaaacaagttcacagagaccaacccacaagtgtactaccaggcttccctgaggccctatggagtgactgtttaccattccctcagacagccctatgctaccaatccgtgtaaagataacaatgggggctgtgagcaggtctgtgtcctcagccacagaacagataatgatggtttgggtttccgttgcaagtgcacattcggcttccaactggatacagatgagcgccactgcattgctgttcagaatttcctcattttttcatcccaagttgctattcgtgggatcccgttcaccttgtctacccaggaagatgtcatggttccagtttcggggaatccttctttctttgtcgggattgattttgacgcccaggacagcactatctttttttcagatatgtcaaaacacatgatttttaagcaaaagattgatggcacaggaagagaaattctcgcagctaacagggtggaaaatgttgaaagtttggcttttgattggatttcaaagaatctctattggacagactctcattacaagagtatcagtgtcatgaggctagctgataaaacgagacgcacagtagttcagtatttaaataacccacggtcggtggtagttcatccttttgccgggtatctattcttcactgattggttccgtcctgctaaaattatgagagcatggagtgacggatctcacctcttgcctgtaataaacactactcttggatggcccaatggcttggccatcgattgggctgcttcacgattgtactgggtagatgcctattttgataaaattgagcacagcacctttgatggtttagacagaagaagactgggccatatagagcagatgacacatccgtttggacttgccatctttggagagcatttattttttactgactggagactgggtgccattattcgagtcaggaaagcagatggtggagaaatgacagttatccgaagtggcattgcttacatactgcatttgaaatcgtatgatgtcaacatccagactggttctaacgcctgtaatcaacccacgcatcctaacggtgactgcagccacttctgcttcccggtgccaaatttccagcgagtgtgtgggtgcccttatggaatgaggctggcttccaatcacttgacatgcgagggggacccaaccaatgaaccacccacagagcagtgtggcttattttccttcccctgtaaaaatggcagatgtgtgcccaattactatctctgtgatggagtcgatgattgtcatgataacagtgatgagcaactatgtggcacacttaataatacctgttcatcttcggcgttcacctgtggccatggggagtgcattcctgcacactggcgctgtgacaaacgcaacgactgtgtggatggcagtgatgagcacaactgccccacccacgcacctgcttcctgccttgacacccaatacacctgtgataatcaccagtgtatctcaaagaactgggtctgtgacacagacaatgattgtggggatggatctgatgaaaagaactgcaattcgacagagacatgccaacctagtcagtttaattgccccaatcatcgatgtattgacctatcgtttgtctgtgatggtgacaaggattgtgttgatggatctgatgaggttggttgtgtattaaactgtactgcttctcaattcaagtgtgccagtggggataaatgtattggcgtcacaaatcgttgtgatggtgtttttgattgcagtgacaactcggatgaagcaggctgtccaaccaggcctcctggtatgtgccactcagatgaatttcagtgccaagaagatggtatctgcatcccgaacttctgggaatgtgatgggcatccagactgcctctatggatctgatgagcacaatgcctgtgtccccaagacttgcccttcatcatatttccactgtgacaacggaaactgcatccacagggcatggctctgtgatcgggacaatgactgcggggatatgagtgatgagaaggactgccctactcagccctttcgctgtcctagttggcaatggcagtgtcttggccataacatctgtgtgaatctgagtgtagtgtgtgatggcatctttgactgccccaatgggacagatgagtccccactttgcaatgggaacagctgctcagatttcaatggtggttgtactcacgagtgtgttcaagagccctttggggctaaatgcctatgtccattgggattcttacttgccaatgattctaagacctgtgaagacatagatgaatgtgatattctaggctcttgtagccagcactgttacaatatgagaggttctttccggtgctcgtgtgatacaggctacatgttagaaagtgatgggaggacttgcaaagttacagcatctgagagtctgctgttacttgtggcaagtcagaacaaaattattgccgacagtgtcacctcccaggtccacaatatctattcattggtcgagaatggttcttacattgtagctgttgattttgattcaattagtggtcgtatcttttggtctgatgcaactcagggtaaaacctggagtgcgtttcaaaatggaacggacagaagagtggtatttgacagtagcatcatcttgactgaaactattgcaatagattgggtaggtcgtaatctttactggacagactatgctctggaaacaattgaagtctccaaaattgatgggagccacaggactgtgctgattagtaaaaacctaacaaatccaagaggactagcattagatcccagaatgaatgagcatctactgttctggtctgactggggccaccaccctcgcatcgagcgagccagcatggacggcagcatgcgcactgtcattgtccaggacaagatcttctggccctgcggcttaactattgactaccccaacagactgctctacttcatggactcctatcttgattacatggacttttgtgattataatggacaccatcggagacaggtgatagccagtgatttgattatacggcacccctatgccctaactctctttgaagactctgtgtactggactgaccgtgctactcgtcgggttatgcgagccaacaagtggcatggagggaaccagtcagttgtaatgtataatattcaatggccccttgggattgttgcggttcatccttcgaaacaaccaaattccgtgaatccatgtgccttttcccgctgcagccatctctgcctgctttcctcacaggggcctcatttttactcctgtgtttgtccttcaggatggagtctgtctcctgatctcctgaattgcttgagagatgatcaacctttcttaataactgtaaggcaacatataatttttggaatctcccttaatcctgaggtgaagagcaatgatgctatggtccccatagcagggatacagaatggtttagatgttgaatttgatgatgctgagcaatacatctattgggttgaaaatccaggtgaaattcacagagtgaagacagatggcaccaacaggacagtatttgcttctatatctatggtggggccttctatgaacctggccttagattggatttcaagaaacctttattctaccaatcctagaactcagtcaatcgaggttttgacactccacggagatatcagatacagaaaaacattgattgccaatgatgggacagctcttggagttggctttccaattggcataactgttgatcctgctcgtgggaagctgtactggtcagaccaaggaactgacagtggggttcctgccaagatcgccagtgctaacatggatggcacatctgtgaaaactctctttactgggaacctcgaacacctggagtgtgtcactcttgacatcgaagagcagaaactctactgggcagtcactggaagaggagtgattgaaagaggaaacgtggatggaacagatcgaatgatcctggtacaccagctttcccacccctggggaattgcagtccatgattctttcctttattatactgatgaacagtatgaggtcattgaaagagttgataaggccactggggccaacaaaatagtcttgagagataatgttccaaatctgaggggtcttcaagtttatcacagacgcaatgccgccgaatcctcaaatggctgtagcaacaacatgaatgcctgtcagcagatttgcctgcctgtaccaggaggattgttttcctgcgcctgtgccactggatttaaactcaatcctgataatcggtcctgctctccatataactctttcattgttgtttcaatgctgtctgcaatcagaggctttagcttggaattgtcagatcattcagaaaccatggtgccggtggcaggccaaggacgaaacgcactgcatgtggatgtggatgtgtcctctggctttatttattggtgtgattttagcagctcagtggcatctgataatgcgatccgtagaattaaaccagatggatcttctctgatgaacattgtgacacatggaataggagaaaatggagtccggggtattgcagtggattgggtagcaggaaatctttatttcaccaatgcctttgtttctgaaacactgatagaagttctgcggatcaatactacttaccgccgtgttcttcttaaagtcacagtggacatgcctaggcatattgttgtagatcccaagaacagatacctcttctgggctgactatgggcagagaccaaagattgagcgttctttccttgactgtaccaatcgaacagtgcttgtgtcagagggcattgtcacaccacggggcttggcagtggaccgaagtgatggctacgtttattgggttgatgattctttagatataattgcaaggattcgtatcaatggagagaactctgaagtgattcgttatggcagtcgttacccaactccttatggcatcactgtttttgaaaattctatcatatgggtagataggaatttgaaaaagatcttccaagccagcaaggaaccagagaacacagagccacccacagtgataagagacaatatcaactggctaagagatgtgaccatctttgacaagcaagtccagccccggtcaccagcagaggtcaacaacaacccttgcttggaaaacaatggtgggtgctctcatctctgctttgctctgcctggattgcacaccccaaaatgtgactgtgcctttgggaccctgcaaagtgatggcaagaattgtgccatttcaacagaaaatttcctcatctttgccttgtctaattccttgagaagcttacacttggaccctgaaaaccatagcccacctttccaaacaataaatgtggaaagaactgtcatgtctctagactatgacagtgtaagtgatagaatctacttcacacaaaatttagcctctggagttggacagatttcctatgccaccctgtcttcagggatccatactccaactgtcattgcttcaggtatagggactgctgatggcattgcctttgactggattactagaagaatttattacagtgactacctcaaccagatgattaattccatggctgaagatgggtctaaccgcactgtgatagcccgcgttccaaaaccaagagcaattgtgttagatccctgccaagggtacctgtactgggctgactgggatacacatgccaaaatcgagagagccacattgggaggaaacttccgcgtacccattgtgaacagcagtctggtcatgcccagtgggctgactctggactatgaagaggaccttctctactgggtggatgctagtctgcagaggattgaacgcagcactctgacgggcgtggatcgtgaagtcattgtcaatgcagccgttcatgcttttggcttgactctctatggccagtatatttactggactgacttgtacacacaaagaatttaccgagctaacaaatatgacgggtcaggtcagattgcaatgaccacaaatttgctctcccagcccaggggaatcaacactgttgtgaagaaccagaaacaacagtgtaacaatccttgtgaacagtttaatgggggctgcagccatatctgtgcaccaggtccaaatggtgccgagtgccagtgtccacatgagggcaactggtatttggccaacaacaggaagcactgcattgtggacaatggtgaacgatgtggtgcatcttccttcacctgctccaatgggcgctgcatctcggaagagtggaagtgtgataatgacaacgactgtggggatggcagtgatgagatggaaagtgtctgtgcacttcacacctgctcaccgacagccttcacctgtgccaatgggcgatgtgtccaatactcttaccgctgtgattactacaatgactgtggtgatggcagtgatgaggcagggtgcctgttcagggactgcaatgccaccacggagtttatgtgcaataacagaaggtgcatacctcgtgagtttatctgcaatggtgtagacaactgccatgataataacacttcagatgagaaaaattgccctgatcgcacttgccagtctggatacacaaaatgtcataattcaaatatttgtattcctcgcgtttatttgtgtgacggagacaatgactgtggagataacagtgatgaaaaccctacttattgcaccactcacacgtgcagcagcagtgagttccaatgcgcatctgggcgctgtattcctcaacattggtattgtgatcaagaaacagattgttttgatgcctctgatgaacctgcctcttgtggtcactctgagcgaacatgcctagctgatgagttcaagtgtgatggtgggaggtgcatcccaagcgaatggatctgtgacggtgataatgactgtggggatatgagtgacgaggataaaaggcaccagtgtcagaatcaaaactgctcggattccgagtttctctgtgtaaatgacagacctccggacaggaggtgcattccccagtcttgggtctgtgatggcgatgtggattgtactgacggctacgatgagaatcagaattgcaccaggagaacttgctctgaaaatgaattcacctgtggttacggactgtgtatcccaaagatattcaggtgtgaccggcacaatgactgtggtgactatagcgacgagaggggctgcttataccagacttgccaacagaatcagtttacctgtcagaacgggcgctgcattagtaaaaccttcgtctgtgatgaggataatgactgtggagacggatctgatgagctgatgcacctgtgccacaccccagaacccacgtgtccacctcacgagttcaagtgtgacaatgggcgctgcatcgagatgatgaaactctgcaaccacctagatgactgtttggacaacagcgatgagaaaggctgtggcattaatgaatgccatgacccttcaatcagtggctgcgatcacaactgcacagacaccttaaccagtttctattgttcctgtcgtcctggttacaagctcatgtctgacaagcggacttgtgttgatattgatgaatgcacagagatgccttttgtctgtagccagaagtgtgagaatgtaataggctcctacatctgtaagtgtgccccaggctacctccgagaaccagatggaaagacctgccggcaaaacagtaacatcgaaccctatctcatttttagcaaccgttactatttgagaaatttaactatagatggctatttttactccctcatcttggaaggactggacaatgttgtggcattagattttgaccgagtagagaagagattgtattggattgatacacagaggcaagtcattgagagaatgtttctgaataagacaaacaaggagacaatcataaaccacagactaccagctgcagaaagtctggctgtagactgggtttccagaaagctctactggttggatgcccgcctggatggcctctttgtctctgacctcaatggtggacaccgccgcatgctggcccagcactgtgtggatgccaacaacaccttctgctttgataatcccagaggacttgcccttcaccctcaatatgggtacctctactgggcagactggggtcaccgcgcatacattgggagagtaggcatggatggaaccaacaagtctgtgataatctccaccaagttagagtggcctaatggcatcaccattgattacaccaatgatctactctactgggcagatgcccacctgggttacatagagtactctgatttggagggccaccatcgacacacggtgtatgatggggcactgcctcaccctttcgctattaccatttttgaagacactatttattggacagattggaatacaaggacagtggaaaagggaaacaaatatgatggatcaaatagacagacactggtgaacacaacacacagaccatttgacatccatgtgtaccatccatataggcagcccattgtgagcaatccctgtggtaccaacaatggtggctgttctcatctctgcctcatcaagccaggaggaaaagggttcacttgcgagtgtccagatgacttccgcacccttcagctgagtggcagcacctactgcatgcccatgtgctccagcacccagttcctgtgcgctaacaatgaaaagtgcattcctatctggtggaaatgtgatggacagaaagactgctcagatggctctgatgaactggccctttgcccgcagcgcttctgccgactgggacagttccagtgcagtgacggcaactgcaccagcccgcagactttatgcaatgctcaccaaaattgccctgatgggtctgatgaagaccgtcttctttgtgagaatcaccactgtgactccaatgaatggcagtgcgccaacaaacgttgcatcccagaatcctggcagtgtgacacatttaacgactgtgaggataactcagatgaagacagttcccactgtgccagcaggacctgccggccgggccagtttcggtgtgctaatggccgctgcatcccgcaggcctggaagtgtgatgtggataatgattgtggagaccactcggatgagcccattgaagaatgcatgagctctgcccatctctgtgacaacttcacagaattcagctgcaaaacaaattaccgctgcatcccaaagtgggccgtgtgcaatggtgtagatgactgcagggacaacagtgatgagcaaggctgtgaggagaggacatgccatcctgtgggggatttccgctgtaaaaatcaccactgcatccctcttcgttggcagtgtgatgggcaaaatgactgtggagataactcagatgaggaaaactgtgctccccgggagtgcacagagagcgagtttcgatgtgtcaatcagcagtgcattccctcgcgatggatctgtgaccattacaacgactgtggggacaactcagatgaacgggactgtgagatgaggacctgccatcctgaatattttcagtgtacaagtggacattgtgtacacagtgaactgaaatgcgatggatccgctgactgtttggatgcgtctgatgaagctgattgtcccacacgctttcctgatggtgcatactgccaggctactatgttcgaatgcaaaaaccatgtttgtatcccgccatattggaaatgtgatggcgatgatgactgtggcgatggttcagatgaagaacttcacctgtgcttggatgttccctgtaattcaccaaaccgtttccggtgtgacaacaatcgctgcatttatagtcatgaggtgtgcaatggtgtggatgactgtggagatggaactgatgagacagaggagcactgtagaaaaccgacccctaaaccttgtacagaatatgaatataagtgtggcaatgggcattgcattccacatgacaatgtgtgtgatgatgccgatgactgtggtgactggtccgatgaactgggttgcaataaaggaaaagaaagaacatgtgctgaaaatatatgcgagcaaaattgtacccaattaaatgaaggaggatttatctgctcctgtacagctgggttcgaaaccaatgtttttgacagaacctcctgtctagatatcaatgaatgtgaacaatttgggacttgtccccagcactgcagaaataccaaaggaagttatgagtgtgtctgtgctgatggcttcacgtctatgagtgaccgccctggaaaacgatgtgcagctgagggtagctctcctttgttgctactgcctgacaatgtccgaattcgaaaatataatctctcatctgagaggttctcagagtatcttcaagatgaggaatatatccaagctgttgattatgattgggatcccaaggacataggcctcagtgttgtgtattacactgtgcgaggggagggctctaggtttggtgctatcaaacgtgcctacatccccaactttgaatccggccgcaataatcttgtgcaggaagttgacctgaaactgaaatacgtaatgcagccagatggaatagcagtggactgggttggaaggcatatttactggtcagatgtcaagaataaacgcattgaggtggctaaacttgatggaaggtacagaaagtggctgatttccactgacctggaccaaccagctgctattgctgtgaatcccaaactagggcttatgttctggactgactggggaaaggaacctaaaatcgagtctgcctggatgaatggagaggaccgcaacatcctggttttcgaggaccttggttggccaactggcctttctatcgattatttgaacaatgaccgaatctactggagtgacttcaaggaggacgttattgaaaccataaaatatgatgggactgataggagagtcattgcaaaggaagcaatgaacccttacagcctggacatctttgaagaccagttatactggatatctaaggaaaagggagaagtatggaaacaaaataaatttgggcaaggaaagaaagagaaaacgctggtagtgaacccttggctcactcaagttcgaatctttcatcaactcagatacaataagtcagtgcccaacctttgcaaacagatctgcagccacctctgccttctgagacctggaggatacagctgtgcctgtccccaaggctccagctttatagaggggagcaccactgagtgtgatgcagccatcgaactgcctatcaacctgccccccccatgcaggtgcatgcacggaggaaattgctattttgatgagactgacctccccaaatgcaagtgtcctagcggctacaccggaaaatattgtgaaatggcgttttcaaaaggcatctctccaggaacaaccgcagtagctgtgctgttgacaatcctcttgatcgtcgtaattggagctctggcaattgcaggattcttccactatagaaggaccggctcccttttgcctgctctgcccaagctgccaagcttaagcagtctcgtcaagccctctgaaaatgggaatggggtgaccttcagatcaggggcagatcttaacatggatattggagtgtctggttttggacctgagactgctattgacaggtcaatggcaatgagtgaagactttgtcatggaaatggggaagcagcccataatatttgaaaacccaatgtactcagccagagacagtgctgtcaaagtggttcagccaatccaggtgactgtatctgaaaatgtggataataagaattatggaagtcccataaacccttctgagatagttccagagacaaacccaacttcaccagctgctgatggaactcaggtgacaaaatggaatctcttcaaacgaaaatctaaacaaactaccaactttgaaaatccaatctatgcacagatggagaacgagcaaaaggaaagtgttgctgcgacaccacctccatcaccttcgctccctgctaagcctaagcctccttcgagaagagacccaactccaacctattctgcaacagaagacacttttaaagacaccgcaaatcttgttaaagaagactctgaagtatagctataccagctatttagggaataattagaaacacacttttgcacatatattttttacaaacagatgaaaaaagttaacattcagtactttatgaaaaaaatatatttttccctgtttgcctatagttggaggtatcctgtgtgtctttttttacttatgccgtctcatatttttacaaataattatcacaatgtactatatgtatatctttgcactgaagttgtctgaaggtaatactataaatatattgtatatttgtaaattttggaaagattatcctgttactgaatttgctaataaagatgtctgctgatttggttggtgatcattatagtaaatgatccaacaagaaaaggaattgactggggacctttagccgtgtctaaagaagaggcaccactcatatttcctataaaattatctaggaaaggaatccaggccccgctcttgggtccatttttacacattagcacttaattaatgttcaatattacatgtcaatttgattaatggctatgttgataggggccactatgtgttgtatagacatctggacttgactgtagactcctcagataatacagaaggtaggaaaagcaattcagtttggcccttctgtgtgttggcattgtctaaccagaactctctgtttcatgtgtgttctctcactagctgccaagacaacatttttatttgtgatgtctatgaggaaatcccatatcattaagtgccagtgtcctgcattgagtttgtggttaattaaatgagctcttctgctgatggaccctggagcaatttctcccctcacctgacattcaaggtggtcacctgccctagtagttggagctcagtagctgaatttctgaaaccaaatctgtgtcttcataaaataaggtgcaaaaaaaaaaaataccagttaagtaaagcctcaactgggtttttgtttctatgaaaatatcattataatcactatttatttcctaagttgaacctgaatagaaagggaaaccattcttattaagctttttattaggccctgtggctaaatgtgtacatttatattagaatgtactgtacagtccagatcttttctttaattcttattggttttttttttttttttttttttagagatggagtcttgctatattgccaaggctgatcttgaagtcctgggctcaagtgatcctcccacctcagcctcctgagtggttggggttacgggcgtgagccactgtgcctggcttccagctctcctcttaaatagtgggtatagtctgcacaacaggaaccatggcaggaatatacactttcccatagcaaatagcatacctgactctctgtgctaatattgcacatttgttaaacaatgaatgaatggatggatggatggatggatgaatgaatgaaacatatactactgattattttattccagagttctcaaaatatttgttgctgatattttgagtgctgactgtaattactttgattagataaacaactggaaataatgctgctgaaaaagttctaataaatgtgtattttatcaga

SEQ ID NO: 2 - DNA, 4138 bp, Homo sapiens
gggcgccggagctgcggctgctgtagttgtcctagccggtgctggggcggcggggtggcggagcggcgggcgggcgggagggctggcggggcgaacgtctgggagacgtctgaaagaccaacgagactttggagaccagagacgcgcctggggggacctggggcttggggcgtgcgagatttcccttgcattcgctgggagctcgcgcagggatcgtcccatggccggggctcggagccgcgacccttggggggcctccgggatttgctacctttttggctccctgctcgtcgaactgctcttctcacgggctgtcgccttcaatctggacgtgatgggtgccttgcgcaaggagggcgagccaggcagcctcttcggcttctctgtggccctgcaccggcagttgcagccccgaccccagagctggctgctggtgggtgctccccaggccctggctcttcctgggcagcaggcgaatcgcactggaggcctcttcgcttgcccgttgagcctggaggagactgactgctacagagtggacatcgaccagggagctgatatgcaaaaggaaagcaaggagaaccagtggttgggagtcagtgttcggagccaggggcctgggggcaagattgttacctgtgcacaccgatatgaggcaaggcagcgagtggaccagatcctggagacgcgggatatgattggtcgctgctttgtgctcagccaggacctggccatccgggatgagttggatggtggggaatggaagttctgtgagggacgcccccaaggccatgaacaatttgggttctgccagcagggcacagctgccgccttctcccctgatagccactacctcctctttggggccccaggaacctataattggaaggggttgctttttgtgaccaacattgatagctcagaccccgaccagctggtgtataaaactttggaccctgctgaccggctcccaggaccagccggagacttggccctcaatagctacttaggcttctctattgactcggggaaaggtctggtgcgtgcagaagagctgagctttgtggctggagccccccgcgccaaccacaagggtgctgtggtcatcctgcgcaaggacagcgccagtcgcctggtgcccgaggttatgctgtctggggagcgcctgacctccggctttggctactcactggctgtggctgacctcaacagtgatggctggccagacctgatagtgggtgccccctacttctttgagcgccaagaagagctggggggtgctgtgtatgtgtacttgaaccaggggggtcactgggctgggatctcccctctccggctctgcggctcccctgactccatgttcgggatcagcctggctgtcctgggggacctcaaccaagatggctttccagatattgcagtgggtgccccctttgatggtgatgggaaagtcttcatctaccatgggagcagcctgggggttgtcgccaaaccttcacaggtgctggagggcgaggctgtgggcatcaagagcttcggctactccctgtcaggcagcttggatatggatgggaaccaataccctgacctgctggtgggctccctggctgacaccgcagtgctcttcagggccagacccatcctccatgtctcccatgaggtctctattgctccacgaagcatcgacctggagcagcccaactgtgctggcggccactcggtctgtgtggacctaagggtctgtttcagctacattgcagtccccagcagctatagccctactgtggccctggactatgtgttagatgcggacacagaccggaggctccggggccaggttccccgtgtgacgttcctgagccgtaacctggaagaacccaagcaccaggcctcgggcaccgtgtggctgaagcaccagcatgaccgagtctgtggagacgccatgttccagctccaggaaaatgtcaaagacaagcttcgggccattgtagtgaccttgtcctacagtctccagacccctcggctccggcgacaggctcctggccaggggctgcctccagtggcccccatcctcaatgcccaccagcccagcacccagcgggcagagatccacttcctgaagcaaggctgtggtgaagacaagatctgccagagcaatctgcagctggtccgcgcccgcttctgtacccgggtcagcgacacggaattccaacctctgcccatggatgtggatggaacaacagccctgtttgcactgagtgggcagccagtcattggcctggagctgatggtcaccaacctgccatcggacccagcccagccccaggctgatggggatgatgcccatgaagcccagctcctggtcatgcttcctgactcactgcactactcaggggtccgggccctggaccctgcggagaagccactctgcctgtccaatgagaatgcctcccatgttgagtgtgagctggggaaccccatgaagagaggtgcccaggtcaccttctacctcatccttagcacctccgggatcagcattgagaccacggaactggaggtagagctgctgttggccacgatcagtgagcaggagctgcatccagtctctgcacgagcccgtgtcttcattgagctgccactgtccattgcaggaatggccattccccagcaactcttcttctctggtgtggtgaggggcgagagagccatgcagtctgagcgggatgtgggcagcaaggtcaagtatgaggtcacggtttccaaccaaggccagtcgctcagaaccctgggctctgccttcctcaacatcatgtggcctcatgagattgccaatgggaagtggttgctgtacccaatgcaggttgagctggagggcgggcaggggcctgggcagaaagggctttgctctcccaggcccaacatcctccacctggatgtggacagtagggataggaggcggcgggagctggagccacctgagcagcaggagcctggtgagcggcaggagcccagcatgtcctggtggccagtgtcctctgctgagaagaagaaaaacatcaccctggactgcgcccggggcacggccaactgtgtggtgttcagctgcccactctacagctttgaccgcgcggctgtgctgcatgtctggggccgtctctggaacagcacctttctggaggagtactcagctgtgaagtccctggaagtgattgtccgggccaacatcacagtgaagtcctccataaagaacttgatgctccgagatgcctccacagtgatcccagtgatggtatacttggaccccatggctgtggtggcagaaggagtgccctggtgggtcatcctcctggctgtactggctgggctgctggtgctagcactgctggtgctgctcctgtggaagatgggattcttcaaacgggcgaagcaccccgaggccaccgtgccccagtaccatgcggtgaagattcctcgggaagaccgacagcagttcaaggaggagaagacgggcaccatcctgaggaacaactggggcagcccccggcgggagggcccggatgcacaccccatcctggctgctgacgggcatcccgagctgggccccgatgggcatccagggccaggcaccgcctaggttcccatgtcccagcctggcctgtggctgccctccatcccttccccagagatggctccttgggatgaagagggtagagtgggctgctggtgtcgcatcaagatttggcaggatcggcttcctcaggggcacagacctctcccacccacaagaactcctcccacccaacttccccttagagtgctgtgagatgagagtgggtaaatcagggacagggccatggggtagggtgagaagggcaggggtgtcctgatgcaaaggtggggagaagggatcctaatcccttcctctcccattcaccctgtgtaacaggaccccaaggacctgcctccccggaagtgccttaacctagagggtcggggaggaggttgtgtcactgactcaggctgctccttctctagtttcccctctcatctgaccttagtttgctgccatcagtctagtggtttcgtggtttcgtctatttattaaaaaatatttgagaacaaaaaaaaaaaaaaaaaa

SEQ ID NO: 3 - DNA, 2811 bp, Homo sapiens
ccatttcaatagtcgcgggatacttgaactgcaagaacagccgccgctccggcgggctgctcgctgcatctctgggcgtctttggctcgccacgctgggcagtgcctgcctgcgcctttcgcaacctcctcggccctgcgtggtctcgagctgggtgagcgagcgggcgggctggtaggctggcctgggctgcgaccggcggctacgactattctttggccgggtcggtgcgagtggtcggctgggcagagtgcacgctgcttggcgccgcaggctgatcccgccgtccactcccgggagcagtgatgttgggcaactctgcgccggggcctgcgacccgcgaggcgggctcggcgctgctagcattgcagcagacggcgctccaagaggaccaggagaatatcaacccggaaaaggcagcgcccgtccaacaaccgcggacccgggccgcgctggcggtactgaagtccgggaacccgcggggtctagcgcagcagcagaggccgaagacgagacgggttgcaccccttaaggatcttcctgtaaatgatgagcatgtcaccgttcctccttggaaagcaaacagtaaacagcctgcgttcaccattcatgtggatgaagcagaaaaagaagctcagaagaagccagctgaatctcaaaaaatagagcgtgaagatgccctggcttttaattcagccattagtttacctggacccagaaaaccattggtccctcttgattatccaatggatggtagttttgagtcaccacatactatggacatgtcaattgtattagaagatgaaaagccagtgagtgttaatgaagtaccagactaccatgaggatattcacacataccttagggaaatggaggttaaatgtaaacctaaagtgggttacatgaagaaacagccagacatcactaacagtatgagagctatcctcgtggactggttagttgaagtaggagaagaatataaactacagaatgagaccctgcatttggctgtgaactacattgataggttcctgtcttccatgtcagtgctgagaggaaaacttcagcttgtgggcactgctgctatgctgttagcctcaaagtttgaagaaatataccccccagaagtagcagagtttgtgtacattacagatgatacctacaccaagaaacaagttctgagaatggagcatctagttttgaaagtccttacttttgacttagctgctccaacagtaaatcagtttcttacccaatactttctgcatcagcagcctgcaaactgcaaagttgaaagtttagcaatgtttttgggagaattaagtttgatagatgctgacccatacctcaagtatttgccatcagttattgctggagctgcctttcatttagcactctacacagtcacgggacaaagctggcctgaatcattaatacgaaagactggatataccctggaaagtcttaagccttgtctcatggaccttcaccagacctacctcaaagcaccacagcatgcacaacagtcaataagagaaaagtacaaaaattcaaagtatcatggtgtttctctcctcaacccaccagagacactaaatctgtaacaatgaaagactgcctttgttttctaagatgtaaatcactcaaagtatatggtgtacagtttttaacttaggttttaattttacaatcatttctgaatacagaagttgtggccaagtacaaattatggtatctattactttttaaatggttttaatttgtatatcttttgtatatgtatctgtcttagatatttggctaattttaagtggttttgttaaagtattaatgatgccagctgtcaggataataaattgatttggaaaactttgcaagtcaaatttaacttcttcaggattttgcttagtaaagaagtttacttggtttactatataatgggaagtgaaaagccttcctctaaaattaaagtaggtttaggaaaacagaccctcaaattctgacattcattttcctaagcaactggatcaatttgctgacttgggcataatctaatctaagcatatctgaatacagtattcagagatagatacagtagagattccccagactttttcgctctttgtaaaacctgtttgtttaggttttgcgaggtaaactcaacagaggttgggagtggaagagggtgggaagcttatatgcaaattaacagacgagaaatgctccagaaggtttattattttaaagcacattaaaaacaaaaaactatttttaaaatcctgctagattttataatggatttgtgaataaaaaatacccagggttctcagaatggaataaatatcccttttaatagttatatatacagatatacaactgttagctttaattggcagctctcttcttttttcttcttttcactggctttttacttggtgctttttcttgttttgcactggtggtctgtgttctgtgaataaagcaaagtaagaatttactaagagtatgttaagttttggattattgaaataagaggcatttcttagttttccagtaggatctaaaatgtgtcagctatgagtaagactggcatccaagaagtttatattatagatttaggtcctaatttttataaatcacaaggtaaaaaaatcacagaacagatggatctctaatgaaaaagggatgtctttttgtttatagtcatgtggcaagatgagagtaaaaccagagagcaaacctctataagtgttgagtatatgtatacatttgaaataaaccagaaatttgttaccttaaaaaaaaaaaaaa

SEQ ID NO: 4 - DNA, 12701 bp, Homo sapiens
cctcccgcctcagttcgcgccgcgcctcggcttggaacgcaggagcgccggctccgggagcccgagcggagccagccgcgcgcacagccagcggccgcgccggcgatgcggggccaccccgcgcccgccccagtcccggccccggcccccgcgggaaggggctgagctgcccgccgccgcccggatggcgagcctcgccgcgctcgccctcagcctgctcctgaggctgcagctgccgccactgcccggcgcccgggctcagagcgccgcaggtggctgttcctttgatgagcactacagcaactgtggttatagtgtggctctagggaccaatgggttcacctgggagcagattaacacatgggagaaaccaatgctggaccaggcagtgcccacaggatctttcatgatggtgaacagctctgggagagcctctggccagaaggcccaccttctcctgccaaccctgaaggagaatgacacccactgcatcgacttccattactacttctccagccgtgacaggtccagcccaggggccttgaacgtctacgtgaaggtgaatggtggcccccaagggaaccctgtgtggaatgtgtccggggtcgtcactgagggctgggtgaaggcagagctcgccatcagcactttctggccacatttctatcaggtgatatttgaatccgtctcattgaagggtcatcctggctacatcgccgtggacgaggtccgggtccttgctcatccatgcagaaaagcacctcattttctgcgactccaaaacgtggaggtgaatgtggggcagaatgccacatttcagtgcattgctggtgggaagtggtctcagcatgacaagctttggctccagcaatggaatggcagggacacggccctgatggtcacccgtgtggtcaaccacaggcgcttctcagccacagtcagtgtggcagacactgcccagcggagcgtcagcaagtaccgctgtgtgatccgctctgatggtgggtctggtgtgtccaactacgcggagctgatcgtgaaagagcctcccacgcccattgctcccccagagctgctggctgtgggggccacatacctgtggatcaagccaaatgccaactccatcatcggggatggccccatcatcctgaaggaagtggaatatcgcaccaccacaggcacgtgggcagagacccacatagtcgactctcccaactataagctgtggcatctggaccccgatgttgagtatgagatccgagtgctcctcacacgaccaggtgaggggggtacgggaccgccagggcctcccctcaccaccaggaccaagtgtgcagatccggtacatggcccacagaacgtggaaatcgtagacatcagagcccggcagctgaccctgcagtgggagcccttcggctacgcggtgacccgctgccatagctacaacctcaccgtgcagtaccagtatgtgttcaaccagcagcagtacgaggccgaggaggtcatccagacctcctcccactacaccctgcgaggcctgcgccccttcatgaccatccggctgcgactcttgctgtctaaccccgagggccgaatggagagcgaggagctggtggtgcagactgaggaagacgttccaggagctgttcctctagaatccatccaaggggggccctttgaggagaagatctacatccagtggaaacctcccaatgagaccaatggggtcatcacgctctacgagatcaactacaaggctgtcggctcgctggacccaagtgctgacctctcgagccagagggggaaagtgttcaagctccggaatgaaacccaccacctctttgtgggtctgtacccagggaccacctattccttcaccatcaaggccagcacagcaaagggctttgggccccctgtcaccactcggattgccaccaaaatttcagctccatccatgcctgagtacgacacagacaccccattgaatgagacagacacgaccatcacagtgatgctgaaacccgctcagtcccggggagctcctgtcagtgtttatcagctggttgtcaaggaggagcgacttcagaagtcacggagggcagctgacattattgagtgcttttcggtgcccgtgagctatcggaatgcctccagcctcgattctctacactactttgctgctgagttgaagcctgccaacctgcctgtcacccagccatttacagtgggtgacaataagacatacaatggctactggaaccctcctctctctcccctgaaaagctacagcatctacttccaggcactcagcaaagccaatggagagaccaaaatcaactgtgttcgtctggctacaaaagcaccaatgggcagcgcccaggtgaccccggggactccactctgcctcctcaccacaggtgcctccacccagaattctaacactgtggagccagagaagcaggtggacaacaccgtgaagatggctggcgtgatcgctggcctcctcatgttcatcatcattctcctgggcgtgatgctcaccatcaaaaggagaagaaatgcttattcctactcctattacttgaagctggccaagaagcagaaggagacccagagtggagcccagagggagatggggcctgtggcctctgccgacaaacccaccaccaagctcagcgccagccgcaatgatgaaggcttctcttctagttctcaggacgtcaacggattcacagatggcagccgcggggagctttcccagcccaccctcacgatccagactcatccctaccgcacctgtgaccctgtggagatgagctacccccgggaccagttccaacccgccatccgggtggctgacttgctgcagcacatcacgcagatgaagagaggccagggctacgggttcaaggaggaatacgaggccttaccagaggggcagacagcttcgtgggacacagccaaggaggatgaaaaccgcaataagaatcgatatgggaacatcatatcctacgaccattcccgggtgaggctgctggtgctggatggagacccgcactctgactacatcaatgccaactacattgacggataccatcgacctcggcactacattgcgactcaaggtccgatgcaggagactgtaaaggacttttggagaatgatctggcaggagaactccgccagcatcgtcatggtcacaaacctggtggaagtgggcagggtgaaatgtgtgcgatactggccagatgacacggaggtctacggagacattaaagtcaccctgattgaaacagagcccctggcagaatacgtcatacgcaccttcacagtccagaagaaaggctaccatgagatccgggagctccgcctcttccacttcaccagctggcctgaccacggcgttccctgctatgccactggccttctgggcttcgtccgccaggtcaagttcctcaaccccccggaagctgggcccatagtggtccactgcagtgctggggctgggcggactggctgcttcattgccattgacaccatgcttgacatggccgagaatgaaggggtggtggacatcttcaactgcgtgcgtgagctccgggcccaaagggtcaacctggtacagacagaggagcaatatgtgtttgtgcacgatgccatcctggaagcgtgcctctgtggcaacactgccatccctgtgtgtgagttccgttctctctactacaatatcagcaggctggacccccagacaaactccagccaaatcaaagatgaatttcagaccctcaacattgtgacaccccgtgtgcggcccgaggactgcagcattgggctcctgccccggaaccatgataagaatcgaagtatggacgtgctgcctctggaccgctgcctgcccttccttatctcagtggacggagaatccagcaattacatcaacgcagcactgatggatagccacaagcagcctgccgccttcgtggtcacccagcaccctctacccaacaccgtggcagacttctggaggctggtgttcgattacaactgctcctctgtggtgatgctgaatgagatggacactgcccagttctgtatgcagtactggcctgagaagacctccgggtgctatgggcccatccaggtggagttcgtctccgcagacatcgacgaggacatcatccacagaatattccgcatctgtaacatggcccggccacaggatggttatcgtatagtccagcacctccagtacattggctggcctgcctaccgggacacgcccccctccaagcgctctctgctcaaagtggtccgacgactggagaagtggcaggagcagtatgacgggagggagggacgtactgtggtccactgcctaaatgggggaggccgtagtggaaccttctgtgccatctgcagtgtgtgtgagatgatccagcagcaaaacatcattgacgtgttccacatcgtgaaaacactgcgtaacaacaaatccaacatggtggagaccctggaacagtataaatttgtatacgaggtggcactggaatatttaagctccttttagctcaatgggatggggaacctgccggagtccagaggctgctgtgaccaagcccccttttgtgtgaatggcagtaactgggctcaggagctctgaggtggcaccctgcctgactccaaggagaagactggtggccctgtgttccacggggggctctgcaccttctgaggggtctcctgttgccgtgggagatgctgctccaaaaggcccaggcttccttttcaacctaaccagccacagccaagggcccaagcagaagtacacccacaagcaaggccttggatttctggctcccagaccacctgcttttgttctgagtttgtggatctcttggcaagccaactgtgcaggtgctggggagtgggaggctcccctgccctccttctccttaggagtggaggagatgtgtgttctgctcctctacgtcatggaaaagattgaggctcttgggggtcactgctctgctgccccctgcaacctccttcaggggcctctggcaccagacatttgcagtctggaccagtgtgaccttacgatgttccctaggccacaagagaggccccccatcctcacacctaacctgcatggggcttcgcccacaaccattctgtaccccttccccagcctgggccttgaccgtccagcattcactggccggccagctgtgtccacagcagtttttgataaaggtgttctttgcttttttgtgtggtcagtgggagggggtggaactgcagggaacttctctgctcctccttgtctttgtaaaaagggaccacctccctggggcagggcttgggctgacctgtaggatgtaacccctgtgtttctttggtggtagctttctttggaagagacaaacaagataagatttgattattttccaaagtgtatgtgaaaagaaactttcttttggagggtgtaaaatcttagtctcttatgtcaaaaagaagggggcgggggagtttgagtatgtacctctaagacaaatctctcgggccttttattttttcctggcaatgtccttaaaagctcccaccctgggacagcatgccactgagcaaggagagatgggtgagcctgaagatggtccctttggtttctggggcaaatagagcaccagctttgtgcataatttggatgtccaaatttgaactccttcctaaagaaacccagcagccaccttgaaaaaggccattgtggagcccattatactttgatttaaaataggccaagagaatcaggcctggagatctagggtcttgtccaaagtgtgagtgagtcaatgagagggaaccaacatttgctaagtctctactgtatgccagggatcatgcttggcactttccataggacatttcacacagtccttagaacccccaggagagagctactgacttgttatcatctccatttgatcatctcctccaatgaggaaacccacgcaccttccttagtaatgaaatcctgggttccaaaggggcaggtaatggcaatgagacttctccgtgctgttttcttcatcttctctaagccaagcaattattttatggagggaaaataaggccagaaacttctgagcagataactccacaaatggaaatttagtactttcttcctgatgccagttcttctgggaagcgcagaatttcagatatattttagtaacacattcccagctccccaggaaagccagtctcatctaatttcttagtcagtaaaaacaattccctgttccttcaggctatgaatggaccagccagggaaactctcgaccttgatctctagccagtgcttaggcccaatatctgacagcctcaggtgggctgggacctaggaagctccatcttgaaggctggtctagccccagacagggcatgaggggcagagaattcaagaaggtacagctttggccctcaagagcccactgtatgctggggaaatggaaccatggtgcagtagtgtggagtggatgagtgttccatgagcctaggagcaagaaagtctcttcggcctcgggcttcctggagaaggggacgtccattcctgctgggtcttaacaagcataaaaaggaaaaaaaggaaactcaggcaaagggatccatatgtgcaatggcaaagaaatgtgaaaaggcattgggagaagcagtctgggggaggccagcccagtgcgggcacagcacaacacggggagcagcaagagatgagccagggtccaggagacagatgcccatcgcgagtacagactttgtcctattggcaacaaggagtccatggagctttagagagatgcactcagcttcgtgttggccaagactccttctgggccaatggggctgcctcttttcctttcatcagacactgtgaaaacattcccttaagcgtgcactttttaatatcacatctatttgtctgtctgctcattgttttgttgctggaactaaatatgcaatggatcatgagactcagattctatgagaaacccagggtctctgctttaccacggagcagggtcaccaacccagatctccaggcccatgaggatggaacatgaaaggagccgacaaaagttgcttccattggcatgggctctggagctgtccagaagtccagggacaccagacttgatcaaggaagggctgtcactttagaggttcaaaaggaagtgcctcaaagcaaaggcaagcaaaggaaccccacgatgaacttgctcttttcctttgatgagcctctccccaggtgtatttcagcagaccccggggacccacccccactgggcctgctggcctccctcggctccagcccaatgccccagctggccttccccagcctgcaaggagcctgtagcatggcaaatctgcctgctgtatgctattttcttagatcttggtacatccagacaggatgagggtggagggagagctatttaacacaaatcctaagatttttttctgctcaggaaggggtgaaatagctggcagatacaaaagacagtggcttttatcattttaaatggtaggaatttaaggtgtgacttcagggagaaacaaacttgcaaaaaaaaaaaatctcaggccatgttggggtaacccagcaagggccagtgatgatttcccccagctcatccccttattttcccacaacccaaccattctctaaagcaggacagtgaataggtcttaggccagtgcacacaggaagaaattgaggcttatggatggggatgacttccctaagatcccatgggacaaggatgtggcaaggcttggatgagatggggcaccagtgcccaggaatttgaacattttcctttacccaggaaatctccggagccaacaccaccacccccagggggtctccccaccccaccccatttacagggtgagctcagcctgtcatgagcagaggaaaatattattaatgctctctgagtctttacaacaggagctcttacctcatagatgtgggctctgtttggggaagatgcaaggaagtaatgagaagcccaggaaatttctccacctgtgtttatggcctaaatagcttcaggatgtatcttagctgcactccaacattgcatcctttctggggtgaagaatctgggccaaccaggggtccttgggcctctagaaggccacagtaggcctctctttgtgggaatggaaggggacagtttgcttttagtgctggccctctctgtgggtgtggcctgcaaaggaaccaacagaccctatgctggggactctaacatgtgagctcattaaattcttccagcattctaaaggagggtttgtgattgtcaccatttactgatgaggaaactaaggctcctaggggagaaatcacttgcccacagttccacagctagtgagtgaatgaaccaggatttaaaccggttttttctcactacagagacaatatttttccaccattgtatctcacatttttcccaggaggttacccataacagaagagactagagtggaacagatacgtcagtggataaagctcaaagcaaacaacagtaagcttaaaattccttcatagtctcatgttttacgttcacaattcatgcaaaatttgcattccactttctgatttagccttgttggttttaatatgactctatgaatatttcaaaaaaaaatgtgctctgttcctcatgttgttctgttctgttcaccccgctatgacggaccctaggtcagctggtcttcagcttgaccctagaattgactctaggagcagtgaccctgctgcctcccagagccagttataggctcaagatcaagaccaactgaccttctcctaggcagctcctttggtgtgtgggtgctctgacctcactgttcatgaggggacctcaactaaggcatcttccagttgggtgctggaaggaacccattaactcacactagaatgatgaggatttgctcatctggcgtggagaaggatgagcccacaaaaccctaaagggaaaagagaagctggacacagctgtactcagcagattcctgaatgctaggctggaaagtggtgcctgttgtccaagtggagtcacatggttgctaatgtgggcaagtctgaggacacacttcatgagcagctggggtctggaaggctcctcactttaccctagccacacataattactgggtgcctacagcacctagcaccttggagggggcactattaggaaatcgagattactatggcacaattaattcctgggtaaggcatggggttgtggtggacagagctcagtctttagtttgaacgaaaacatacatacatgaaaaacatacatgaaaaaaggaccctcatcaacattagaaggggtagatttggagcactttaggcaggaaaacaggaacgcaaggccaggaaactggaacccagtgaatactcagaaccgaggatgcagatgacttatttagcaaaatggtcacttctgtgacatagctggagaaaggatgggtaacagcttgccagagccacttggaacaagggcaaatctcagtgtctggggcaaaagatgatgcatttccctctgacccatcatgtttattcatcctccactccccattgccacactagctcttgctgtaagtcctcaccaggatctacatttcctcgtcgctggtgggaaccccttagagtacatagaggtatcagtccagtaagactgctctacacaacagaagtgaggcccagggagtagcagccaggcccttatcctgttacctctgcaggagtgactgcccaacccagatccagagacattgaaggaaatgataattccttggtacctcactgccttgggacaaaatgaagaaagccacccttccttaggctgcagcttgccactcctgggctgggtaaacaggtcatcagcaccaagctcaaccaggagtaacactctggaagacatgggtgagcccaagaggaagcatgaacaggacgctgttcctaagtcatgtcaacaggttgtgctgggccaggatccccagggaaaaaaatggtcaacccaactggagggtaggttagaagaaaaaaaacataaacgtggatagtcatgtcatctcaaatccctgacttggcttccccattacttaacagtctgagctccttcttagcctgtgaccagcttcaaatcacagccaagtaaaacaaggaaataggaaaagtaaatccaactagaagagacaagctgagattcagatttgtttactcctcccatgcaaagtttccctgttggaggttttccatgtatacatgtctagaagtgatagaatgcaaggccttggctttgtcttgcagggatctgcctttgaggtcatagactgaacagcagggagagaggttagtggtggagtgtggggggagctgttctagctccagtttcttctgacacatttttcaggatcatggatctgatcctccgaagcacagcagagatatctaagccatatttgtgcacatgagcagactcttctagttttttagtaaccagggatgggcttttgcatggcactgactatagagatgtcttgtagagatcaagccagtcttttgcatcccacctgcccacctccagaagagatgggaaaaggtcatcaaagggcattcaccaactgaaatccactcatgaatgttaggtctctaaaaggaggcatcaacactcacaatggtagcctccaaacctagcatcccacctatctaagagctcaggggtggtccactggggcagatacaagggaagtgcaagggctcaggatgaaagaaaatctattgggaagagttttaggggcttgatcattatggggcttccttctatatctgagaactgctctgggtggtgagatgtggactctgatccttaattggaatgttcggagaatgagtgtctggtggccttgaagtgttggacagaaaagtatcagtataaaagcctggagctcagggtaattaatgtagttcatggttccttagtgagcaggactcttggatgtggaggagaaagggtcataggaagtaaaccaccaaaattacaaaattgagtctctgtacaattacttcagtgcctttgggcttatgaatacaaatcagtgggccttctctatgatggtccaacaaactctcagtgtccaccctgtccctgtatctcccatggaagatgaataatgtcaggtgttctttgggtcaaaggccccagggcagtctggaggcttagagggcagagtggtgtcattccatgtaaagttaggcttctgaggggtcaggcagaatatggtgtccatatcttccatagctctgcagattcttggatgaagtcaagcacagtttgctagacccaggtcactcctctgagtataactaggacccatgagtgaaacttaatagctgtaaggaagaacctgctgtctgccagagaggataagctgcccatctcagcagctgtctaaaagaaggcaggtgtctctttaaagggaagagaagcattggtgaaatggatttcaggtcacttccattccagatgggtgagatcttgtggagctgggatcatgtttgaactcattcatacctgtagagcacgaatccaagtagattgtgtttggtctgtacaggctgaagccccctgctctcccacccaagtgcccccactgagcaggccaacatgctgttgtggccacatatactgggctgatccaggctggttatcaccaaacagcaaaccatagggaacagctgctttgccatagacccaatacccatgtagatctctcatgagagcagccataactcagacccactgaccaacagggccatgagtgacagccagaaccagtgaaggtccaagtaggacacagagcagggcttttcttaccatacacattatctccagaggttatttctaccccactccctattcaaggcctgttggagcacactgcaaaagcaaaagcacagtaactcaatttacacatgattataatcatttccagtgcacacatttcatcaccaggtggatcctgagctagcccatgtaaatccgggttaacccatattggtaatcatactcaaaagcacttttcaccctacattctactagccaatcaaagacaaagagttgtggcctctaccattgccttggcttctggacaccctcacaagctatcccaaggttcccgctcaactccagggaggctgacatcttcacatccactgggcatataatattgcatgagaccaaagtctccacactctttgcagcctcctccatgaatcccaatggcctgcacttgtacagtttgggtgtttgatagataaagcacgtatgagaagagaaaacaaaataaatcaactttttaaaaaagccagcactgtgctgtcaatgttttttttttcttttcaattctagctcagaaaagcagaaggtaaataatgtcaggtcaatgaatatcagatatattttttgactgtacattacagtgaagtgtaatctttttacacctgcaagtccatcttatttattcttgtaaatgttccctgacaatgtttgtaatatggctgtgttaaaaaatctatacaataaagctgtgaccctgagattcatgttttcctaagataaaaaaaa

SEQ ID NO: 5 - DNA, 822 bp, Homo sapiens
  misc_feature (566)..(566): n is a, c, g, or t
aaaaaaaaaagtcctgtggaaatcatatagacaaacatttgcaaagctgctactgccattgtaccagtgttaaactgtgttctaccttgcatcttttactgatttttatgacagattttatattggtaaccattcgagaactctgtaagtgctatggcttccttaaactacgatttatcatatgctcccagtgtttactttgagactgaatggcaaccagagaatgtaaacaaccaaggtgcatctggttatgttttaaaataaagattaataaaagttaaggtaaaaggtctgtgtctgaacctatgcattttttcacctctgaggaggatactaatacctaatgagaaaagctgaaatgtgtgggccgcgggtggcgggtgcgctacatggcgctaatggatactctcagacagcttgaggggagggcgcgcaggcaggtcagaaatcgagcctggaggctcggagaagaattcggagttacccaggggcgtgggaccctaagcgagtggagtggaacacccttgattctcgtgagtggagaggtgtcaccaaaaatattgagnccggggcggactattaggacagaactaccatgatcatatccacgaggtgctaaactggtgcgacaagggtctgaaggcgcagtaaatcagaggcgtggtaacaccccgctaaggagtgggccgtaagtgtcctggcgcctgggatgtccagagaagattcgggggggccaataagggcaagtaacccaggattctggcaggcaacactaacttcagttcgaaccgggttccccggtggcggttacaacacaagccc

SEQ ID NO: 6 - DNA, 567 bp, Homo sapiens
ccgggcgcgttccgttggcggcggattcgaacgttcggactgaggtttttctgcctgaagaagcgtcatacggaccggattgttttcgctggcccagtgtccccggagcttgtgtgcgatacagagagcacctcggaagctgaggcagctggtacttgacagagaggatggcgctgtcgaccatagtctcccagaggaagcagataaagcggaaggctccccgtggctttctaaagcgagtcttcaagcgaaagaagcctcaacttcgtctggagaaaagtggtgacttattggtccatctgaactgtttactgtttgttcatcgattagcagaagagtccaggacaaacgcttgtgcgagtaaatgtagagtcattaacaaggagcatgtactggccgcagcaaaggtaattctaaagaagagcagaggttagaagtcaaagaacatattcttgaaagttatgatgcattcttttgggtggtaacagatcataaagacattttttacacatcagttaatatgggattattaaatattggctataagtgaaaaaaaaaaaaaaaaaa

SEQ ID NO: 7 - DNA, 2501 bp, Homo sapiens
cgaaaagattcttaggaacgccgtaccagccgcgtctctcaggacagcaggcccctgtccttctgtcgggcgccgctcagccgtgccctccgcccctcaggttctttttctaattccaaataaacttgcaagaggactatgaaagattatgatgaacttctcaaatattatgaattacatgaaactattgggacaggtggctttgcaaaggtcaaacttgcctgccatatccttactggagagatggtagctataaaaatcatggataaaaacacactagggagtgatttgccccggatcaaaacggagattgaggccttgaagaacctgagacatcagcatatatgtcaactctaccatgtgctagagacagccaacaaaatattcatggttcttgagtactgccctggaggagagctgtttgactatataatttcccaggatcgcctgtcagaagaggagacccgggttgtcttccgtcagatagtatctgctgttgcttatgtgcacagccagggctatgctcacagggacctcaagccagaaaatttgctgtttgatgaatatcataaattaaagctgattgactttggtctctgtgcaaaacccaagggtaacaaggattaccatctacagacatgctgtgggagtctggcttatgcagcacctgagttaatacaaggcaaatcatatcttggatcagaggcagatgtttggagcatgggcatactgttatatgttcttatgtgtggatttctaccatttgatgatgataatgtaatggctttatacaagaagattatgagaggaaaatatgatgttcccaagtggctctctcccagtagcattctgcttcttcaacaaatgctgcaggtggacccaaagaaacggatttctatgaaaaatctattgaaccatccctggatcatgcaagattacaactatcctgttgagtggcaaagcaagaatccttttattcacctcgatgatgattgcgtaacagaactttctgtacatcacagaaacaacaggcaaacaatggaggatttaatttcactgtggcagtatgatcacctcacggctacctatcttctgcttctagccaagaaggctcggggaaaaccagttcgtttaaggctttcttctttctcctgtggacaagccagtgctaccccattcacagacatcaagtcaaataattggagtctggaagatgtgaccgcaagtgataaaaattatgtggcgggattaatagactatgattggtgtgaagatgatttatcaacaggtgctgctactccccgaacatcacagtttaccaagtactggacagaatcaaatggggtggaatctaaatcattaactccagccttatgcagaacacctgcaaataaattaaagaacaaagaaaatgtatatactcctaagtctgctgtaaagaatgaagagtactttatgtttcctgagccaaagactccagttaataagaaccagcataagagagaaatactcactacgccaaatcgttacactacaccctcaaaagctagaaaccagtgcctgaaagaaactccaattaaaataccagtaaattcaacaggaacagacaagttaatgacaggtgtcattagccctgagaggcggtgccgctcagtggaattggatctcaaccaagcacatatggaggagactccaaaaagaaagggagccaaagtgtttgggagccttgaaagggggttggataaggttatcactgtgctcaccaggagcaaaaggaagggttctgccagagacgggcccagaagactaaagcttcactataatgtgactacaactagattagtgaatccagatcaactgttgaatgaaataatgtctattcttccaaagaagcatgttgactttgtacaaaagggttatacactgaagtgtcaaacacagtcagattttgggaaagtgacaatgcaatttgaattagaagtgtgccagcttcaaaaacccgatgtggtgggtatcaggaggcagcggcttaagggcgatgcctgggtttacaaaagattagtggaagacatcctatctagctgcaaggtataattgatggattcttccatcctgccggatgagtgtgggtgtgatacagcctacataaagactgttatgatcgctttgattttaaagttcattggaactaccaacttgtttctaaagagctatcttaagaccaatatctctttgtttttaaacaaaagatattattttgtgtatgaatctaaatcaagcccatctgtcattatgttactgtcttttttaatcatgtggttttgtatattaataattgttgactttcttagattcacttccatatgtgaatgtaagctcttaactatgtctctttgtaatgtgtaatttctttctgaaataaaaccatttgtgaatataaaaaaaaaaaaaaaaaaaaaaaaaaaaaaaaaaaaaaaaaaaaaaaaaaaaaaaaaaaaaaa

SEQ ID NO: 8 - DNA, 728 bp, Homo sapiens
agggctttctgtatccctaggtttcttgccttgatgtactggagcaatcagatcacacggcggcttggagaaacccagggaccatgggcgcctccaggctctataccctggtgctggtcctgcagcctcagcgagttctcctgggcatgaaaaagcgaggcttcggggccggccggtggaatggctttgggggcaaagtgcaagaaggagagaccatcgaggatggggctaggagggagctgcaggaggagagcggtctgacagtggacgccctgcacaaggtgggccagatcgtgtttgagttcgtgggcgagcctgagctcatggacgtgcatgtcttctgcacagacagcatccaggggacccccgtggagagcgacgaaatgcgcccatgctggttccagctggatcagatccccttcaaggacatgtggcccgacgacagctactggtttccactcctgcttcagaagaagaaattccacgggtacttcaagttccagggtcaggacaccatcctggactacacactccgcgaggtggacacggtctagcgggagcccagggcagcccctgggcaggagacgtggctgctgaacagccgcaaaccatcttcacctgggggcattgagtggcgcagagccgggtttcatctggaattaactggatggaagggaaaataaagctatctagcggtgaaaaaaaaaaaaaaaaaaaaaaaaaaaaaa

SEQ ID NO: 9 - DNA, 818 bp, Homo sapiens
agtcctgtgtccgggccccgaggcacagccagggcaccaggtggagcaccagctacgcgtggcgcagcgcagcgtccctagcaccgagcctcccgcagccgccgagatgctgcgaacagagagctgccgccccaggtcgcccgccggacaggtggccgcggcgtccccgctcctgctgctgctgctgctgctcgcctggtgcgcgggcgcctgccgaggtgctccaatattacctcaaggattacagcctgaacaacagctacagttgtggaatgagatagatgatacttgttcgtcttttctgtccattgattctcagcctcaggcatccaacgcactggaggagctttgctttatgattatgggaatgctaccaaagcctcaggaacaagatgaaaaagataatactaaaaggttcttatttcattattcgaagacacagaagttgggcaagtcaaatgttgtgtcgtcagttgtgcatccgttgctgcagctcgttcctcacctgcatgagagaagaatgaagagattcagagtggacgaagaattccaaagtccctttgcaagtcaaagtcgaggatattttttattcaggccacggaatggaagaaggtcagcagggttcatttaaaatggatgccagctaattttccacagagcaatgctatggaatacaaaatgtactgacattttgttttcttctgaaaaaaatccttgctaaatgtactctgttgaaaatccctgtgttgtcaatgttctcagttgtaacaatgttgtaaatgttcaatttgttgaaaattaaaaaatctaaaaataaa

SEQ ID NO: 10 - DNA, 2748 bp, Homo sapiens
agcgggtgcggggcgggaccggcccggcctatatattgggttggcgccggcgccagctgagccgagcggtagctggtctggcgaggttttatacacctgaaagaagagaatgtcaagacgaagtagccgtttacaagctaagcagcagccccagcccagccagacggaatccccccaagaagcccagataatccaggccaagaagaggaaaactacccaggatgtcaaaaaaagaagagaggaggtcaccaagaaacatcagtatgaaattaggaattgttggccacctgtattatctggggggatcagtccttgcattatcattgaaacacctcacaaagaaataggaacaagtgatttctccagatttacaaattacagatttaaaaatctttttattaatccttcacctttgcctgatttaagctggggatgttcaaaagaagtctggctaaacatgttaaaaaaggagagcagatatgttcatgacaaacattttgaagttctgcattctgacttggaaccacagatgaggtccatacttctagactggcttttagaggtatgtgaagtatacacacttcatagggaaacattttatcttgcacaagacttttttgatagatttatgttgacacaaaaggatataaataaaaatatgcttcaactcattggaattacctcattattcattgcttccaaacttgaggaaatctatgctcctaaactccaagagtttgcttacgtcactgatggtgcttgcagtgaagaggatatcttaaggatggaactcattatattaaaggctttaaaatgggaactttgtcctgtaacaatcatctcctggctaaatctctttctccaagttgatgctcttaaagatgctcctaaagttcttctacctcagtattctcaggaaacattcattcaaatagctcagcttttagatctgtgtattctagccattgattcattagagttccagtacagaatactgactgctgctgccttgtgccattttacctccattgaagtggttaagaaagcctcaggtttggagtgggacagtatttcagaatgtgtagattggatggtaccttttgtcaatgtagtaaaaagtactagtccagtgaagctgaagacttttaagaagattcctatggaagacagacataatatccagacacatacaaactatttggctatgctggaggaagtaaattacataaacaccttcagaaaagggggacagttgtcaccagtgtgcaatggaggcattatgacaccaccgaagagcactgaaaaaccaccaggaaaacactaaagaagataactaagcaaacaagttggaattcaccaagattgggtagaactggtatcactgaactactaaagttttacagaaagtagtgctgtgattgattgccctagccaattcacaagttacactgccattctgattttaaaacttacaattggcactaaagaatacatttaattatttcctatgttagctgttaaagaaacagcaggacttgtttacaaagatgtcttcattcccaaggttactggatagaagccaaccacagtctataccatagcaatgtttttcctttaatccagtgttactgtgtttatcttgataaactaggaattttgtcactggagttttggactggataagtgctaccttaaagggtatactaagtgatacagtactttgaatctagttgttagattctcaaaattcctacactcttgactagtgcaatttggttcttgaaaattaaatttaaacttgtttacaaaggtttagttttgtaataaggtgactaatttatctatagctgctatagcaagctattataaaacttgaatttctacaaatggtgaaatttaatgttttttaaactagtttatttgccttgccataacacattttttaactaataaggcttagatgaacatggtgttcaacctgtgctctaaacagtgggagtaccaaagaaattataaacaagataaatgctgtggctccttcctaactggggctttcttgacatgtaggttgcttggtaataacctttttgtatatcacaatttgggtgaaaaacttaagtaccctttcaaactatttatatgaggaagtcactttactactctaagatatccctaaggaatttttttttttaatttagtgtgactaaggctttatttatgtttgtgaaactgttaaggtcctttctaaattcctccattgtgagataaggacagtgtcaaagtgataaagcttaacacttgacctaaacttctattttcttaaggaagaagagtattaaatatatactgactcctagaaatctatttattaaaaaaagacatgaaaacttgctgtacataggctagctatttctaaatattttaaattagcttttctaaaaaaaaaatccagcctcataaagtagattagaaaactagattgctagtttattttgttatcagatatgtgaatctcttctccctttgaagaaactatacatttattgttacggtatgaagtcttctgtatagtttgtttttaaactaatatttgtttcagtattttgtctgaaaagaaaacaccactaattgtgtacatatgtattatataaacttaaccttttaatactgtttatttttagcccattgtttaaaaaataaaagttaaaaaaatttaactgcttaaaagtaaaaaaaaaaaaaaaaaaa

SEQ ID NO: 11 - DNA, 8630 bp, Homo sapiens
taaatttaaaggcggggcggcctgtgagccctgaagtgccggccgcggagggtcctggccattttcctgggaccagttcagcctgataggatggcggaggaaggagccgtggccgtctgcgtgcgagtgcggccgctgaacagcagagaagaatcacttggagaaactgcccaagtttactggaaaactgacaataatgtcatttatcaagttgatggaagtaaatccttcaattttgatcgtgtctttcatggtaatgaaactaccaaaaatgtgtatgaagaaatagcagcaccaatcatcgattctgccatacaaggctacaatggtactatatttgcctatggacagactgcttcaggaaaaacatataccatgatgggttcagaagatcatttgggagttatacccagggcaattcatgacattttccaaaaaattaagaagtttcctgatagggaatttctcttacgtgtatcttacatggaaatatacaatgaaaccattacagatttactctgtggcactcaaaaaatgaaacctttaattattcgagaagatgtcaataggaatgtgtatgttgctgatctcacagaagaagttgtatatacatcagaaatggctttgaaatggattacaaagggagaaaagagcaggcattatggagaaacaaaaatgaatcaaagaagcagtcgttctcataccatctttaggatgattttggaaagcagagagaagggtgaaccttctaattgtgaaggatctgttaaggtatcccatttgaatttggttgatcttgcaggcagtgaaagagctgctcaaacaggcgctgcaggtgtgcggctcaaggaaggctgtaatataaatcgaagcttatttattttgggacaagtgatcaagaaacttagtgatggacaagttggtggtttcataaattatcgagatagcaagttaacacgaattctccagaattccttgggaggaaatgcaaagacacgtattatctgcacaattactccagtatcttttgatgaaacacttactgctctccagtttgccagtactgctaaatatatgaagaatactccttatgttaatgaggtatcaactgatgaagctctcctgaaaaggtatagaaaagaaataatggatcttaaaaaacaattagaggaggtttctttagagacgcgggctcaggcaatggaaaaagaccaattggcccaacttttggaagaaaaagatttgcttcagaaagtacagaatgagaaaattgaaaacttaacacggatgctggtgacctcttcttccctcacgttgcaacaggaattaaaggctaaaagaaaacgaagagttacttggtgccttggcaaaattaacaaaatgaagaactcaaactatgcagatcaatttaatataccaacaaatataacaacaaaaacacataagctttctataaatttattacgagaaattgatgaatctgtctgttcagagtctgatgttttcagtaacactcttgatacattaagtgagatagaatggaatccagcaacaaagctactaaatcaggagaatatagaaagtgagttgaactcacttcgtgctgactatgataatctggtattagactatgaacaactacgaacagaaaaagaagaaatggaattgaaattaaaagaaaagaatgatttggatgaatttgaggctctagaaagaaaaactaaaaaagatcaagagatgcaactaattcatgaaatttcgaacttaaagaatttagttaagcatgcagaagtatataatcaagatcttgagaatgaactcagttcaaaagtagagctgcttagagaaaaggaagaccagattaagaagctacaggaatacatagactctcaaaagctagaaaatataaaaatggacttgtcatactcattggaaagcattgaagacccaaaacaaatgaagcagactctgtttgatgctgaaactgtagcccttgatgccaagagagaatcagcctttcttagaagtgaaaatctggagctgaaggagaaaatgaaagaacttgcaactacatacaagcaaatggaaaatgatattcagttatatcaaagccagttggaggcaaaaaagaaaatgcaagttgatctggagaaagaattacaatctgcttttaatgagataacaaaactcacctcccttatagatggcaaagttccaaaagatttgctctgtaatttggaattggaaggaaagattactgatcttcagaaagaactaaataaagaagttgaagaaaatgaagctttgcgggaagaagtcattttgctttcagaattgaaatctttaccttctgaagtagaaaggctgaggaaagagatacaagacaaatctgaagagctccatataataacatcagaaaaagataaattgttttctgaagtagttcataaggagagtagagttcaaggtttacttgaagaaattgggaaaacaaaagatgacctagcaactacacagtcgaattataaaagcactgatcaagaattccaaaatttcaaaacccttcatatggactttgagcaaaagtataagatggtccttgaggagaatgagagaatgaatcaggaaatagttaatctctctaaagaagcccaaaaatttgattcgagtttgggtgctttgaagaccgagctttcttacaagacccaagaacttcaggagaaaacacgtgaggttcaagaaagactaaatgagatggaacagctgaaggaacaattagaaaatagagattctacgctgcaaactgtagaaagggagaaaacactgattactgagaaactgcagcaaactttagaagaagtaaaaactttaactcaagaaaaagatgatctaaaacaactccaagaaagcttgcaaattgagagggaccaactcaaaagtgatattcacgatactgttaacatgaatatagatactcaagaacaattacgaaatgctcttgagtctctgaaacaacatcaagaaacaattaatacactaaaatcgaaaatttctgaggaagtttccaggaatttgcatatggaggaaaatacaggagaaactaaagatgaatttcagcaaaagatggttggcatagataaaaaacaggatttggaagctaaaaatacccaaacactaactgcagatgttaaggataatgagataattgagcaacaaaggaagatattttctttaatacaggagaaaaatgaactccaacaaatgttagagagtgttatagcagaaaaggaacaattgaagactgacctaaaggaaaatattgaaatgaccattgaaaaccaggaagaattaagacttcttggggatgaacttaaaaagcaacaagagatagttgcacaagaaaagaaccatgccataaagaaagaaggagagctttctaggacctgtgacagactggcagaagttgaagaaaaactaaaggaaaagagccagcaactccaagaaaaacagcaacaacttcttaatgtacaagaagagatgagtgagatgcagaaaaagattaatgaaatagagaatttaaagaatgaattaaagaacaaagaattgacattggaacatatggaaacagagaggcttgagttggctcagaaacttaatgaaaattatgaggaagtgaaatctataaccaaagaaagaaaagttctaaaggaattacagaagtcatttgaaacagagagagaccaccttagaggatatataagagaaattgaagctacaggcctacaaaccaaagaagaactaaaaattgctcatattcacctaaaagaacaccaagaaactattgatgaactaagaagaagcgtatctgagaagacagctcaaataataaatactcaggacttagaaaaatcccataccaaattacaagaagagatcccagtgcttcatgaggaacaagagttactgcctaatgtgaaagaagtcagtgagactcaggaaacaatgaatgaactggagttattaacagaacagtccacaaccaaggactcaacaacactggcaagaatagaaatggaaaggctcaggttgaatgaaaaatttcaagaaagtcaggaagagataaaatctctaaccaaggaaagagacaaccttaaaacgataaaagaagcccttgaagttaaacatgaccagctgaaagaacatattagagaaactttggctaaaatccaggagtctcaaagcaaacaagaacagtccttaaatatgaaagaaaaagacaatgaaactaccaaaatcgtgagtgagatggagcaattcaaacccaaagattcagcactactaaggatagaaatagaaatgctcggattgtccaaaagacttcaagaaagtcatgatgaaatgaaatctgtagctaaggagaaagatgacctacagaggctgcaagaagttcttcaatctgaaagtgaccagctcaaagaaaacataaaagaaattgtagctaaacacctggaaactgaagaggaacttaaagttgctcattgttgcctgaaagaacaagaggaaactattaatgagttaagagtgaatctttcagagaaggaaactgaaatatcaaccattcaaaagcagttagaagcaatcaatgataaattacagaacaagatccaagagatttatgagaaagaggaacaatttaatataaaacaaattagtgaggttcaggaaaaagtgaatgaactgaaacaattcaaggagcatcgcaaagccaaggattcagcactacaaagtatagaaagtaagatgctcgagttgaccaacagacttcaagaaagtcaagaagaaatacaaattatgattaaggaaaaagaggaaatgaaaagagtacaggaggcccttcagatagagagagaccaactgaaagaaaacactaaagaaattgtagctaaaatgaaagaatctcaagaaaaagaatatcagtttcttaagatgacagctgtcaatgagactcaggagaaaatgtgtgaaatagaacacttgaaggagcaatttgagacccagaagttaaacctggaaaacatagaaacggagaatataaggttgactcagatactacatgaaaaccttgaagaaatgagatctgtaacaaaagaaagagatgaccttaggagtgtggaggagactctcaaagtagagagagaccagctcaaggaaaaccttagagaaactataactagagacctagaaaaacaagaggagctaaaaattgttcacatgcatctgaaggagcaccaagaaactattgataaactaagagggattgtttcagagaaaacaaatgaaatatcaaatatgcaaaaggacttagaacactcaaatgatgccttaaaagcacaggatctgaaaatacaagaggaactaagaattgctcacatgcatctgaaagagcagcaggaaactattgacaaactcagaggaattgtttctgagaagacagataaactatcaaatatgcaaaaagatttagaaaattcaaatgctaaattacaagaaaagattcaagaacttaaggcaaatgaacatcaacttattacgttaaaaaaagatgtcaatgagacacagaaaaaagtgtctgaaatggagcaactaaagaaacaaataaaagaccaaagcttaactctgagtaaattagaaatagagaatttaaatttggctcagaaacttcatgaaaaccttgaagaaatgaaatctgtaatgaaagaaagagataatctaagaagagtagaggagacactcaaactggagagagaccaactcaaggaaagcctgcaagaaaccaaagctagagatctggaaatacaacaggaactaaaaactgctcgtatgctatcaaaagaacacaaagaaactgttgataaacttagagaaaaaatttcagaaaagacaattcaaatttcagacattcaaaaggatttagataaatcaaaagatgaattacagaaaaagatccaagaacttcagaaaaaagaacttcaactgcttagagtgaaagaagatgtcaatatgagtcataaaaaaattaatgaaatggaacagttgaagaagcaatttgaggcccaaaacttatctatgcaaagtgtgagaatggataacttccagttgactaagaaacttcatgaaagccttgaagaaataagaattgtagctaaagaaagagatgagctaaggaggataaaagaatctctcaaaatggaaagggaccaattcatagcaaccttaagggaaatgatagctagagaccgacagaaccaccaagtaaaacctgaaaaaaggttactaagtgatggacaacagcaccttacggaaagcctgagagaaaagtgctctagaataaaagagcttttgaagagatactcagagatggatgatcattatgagtgcttgaatagattgtctcttgacttggagaaggaaattgaattccaaaaagagctttcaatgagagttaaagcaaacctctcacttccctatttacaaaccaaacacattgaaaaactttttactgcaaaccagagatgctccatggaattccacagaatcatgaagaaactgaagtatgtgttaagctatgttacaaaaataaaagaagaacaacatgaatccatcaataaatttgaaatggattttattgatgaagtggaaaagcaaaaggaattgctaattaaaatacagcaccttcaacaagattgtgatgtaccatccagagaattaagggatctcaaattgaaccagaatatggatctacatattgaggaaattctcaaagatttctcagaaagtgagttccctagcataaagactgaatttcaacaagtactaagtaataggaaagaaatgacacagtttttggaagagtggttaaatactcgttttgatatagaaaagcttaaaaatggcatccagaaagaaaatgataggatttgtcaagtgaataacttctttaataacagaataattgccataatgaatgaatcaacagagtttgaggaaagaagtgctaccatatccaaagagtgggaacaggacctgaaatcactgaaagagaaaaatgaaaaactatttaaaaactaccaaacattgaagacttccttggcatctggtgcccaggttaatcctaccacacaagacaataagaatcctcatgttacatcaagagctacacagttaaccacagagaaaattcgagagctggaaaattcactgcatgaagctaaagaaagtgctatgcataaggaaagcaagattataaagatgcagaaagaacttgaggtgactaatgacataatagcaaaacttcaagccaaagttcatgaatcaaataaatgccttgaaaaaacaaaagagacaattcaagtacttcaggacaaagttgctttaggagctaagccatataaagaagaaattgaagatctcaaaatgaagcttgtgaaaatagacctagagaaaatgaaaaatgccaaagaatttgaaaaggaaatcagtgctacaaaagccactgtagaatatcaaaaggaagttataaggctattgagagaaaatctcagaagaagtcaacaggcccaagatacctcagtgatatcagaacatactgatcctcagccttcaaataaacccttaacttgtggaggtggcagcggcattgtacaaaacacaaaagctcttattttgaaaagtgaacatataaggctagaaaaagaaatttctaagttaaagcagcaaaatgaacagctaataaaacaaaagaatgaattgttaagcaataatcagcatctttccaatgaggtcaaaacttggaaggaaagaacccttaaaagagaggctcacaaacaagtaacttgtgagaattctccaaagtctcctaaagtgactggaacagcttctaaaaagaaacaaattacaccctctcaatgcaaggaacggaatttacaagatcctgtgccaaaggaatcaccaaaatcttgtttttttgatagccgatcaaagtctttaccatcacctcatccagttcgctattttgataactcaagtttaggcctttgtccagaggtgcaaaatgcaggagcagagagtgtggattctcagccaggtccttggcacgcctcctcaggcaaggatgtgcctgagtgcaaaactcagtagactcctctttgtcacttctctggagatccagcattccttatttggaaatgactttgtttatgtgtctatccctggtaatgatgttgtagtgcagcttaatttcaattcagtctttactttgccactagagttgaaagataagggaacaggaaatgaatgcattgtggtaatttagaatggtgatagcaataccttcttcttgcatatggtaatacttttaaaagttgaattgttttatttatttgtatattttgtaaagaataaagttattgaaagaaatgtaaagttatctacatgacttagcatattccaaagcataatacatacattaatataaaacatcattttattaacaaaattgtaaatgtttttaataccttacacattcaataaatgtttagtagttctgaatcaccaaaaaaaaaaaaaaaaaa

SEQ ID NO: 12 - DNA, 2319 bp, Homo sapiens
gtggagtttgaattgggtggcggttgactgtagagccgctctctctcactggcacagcgaggttttgctcagcccttgtctcgggaccgcagcctccgccgagcgccatggctcctaggaagggcagtagtcgggtggccaagaccaactccttacggaggcggaagctcgcctcctttctgaaagacttcgaccgtgaagtggaaatacgaatcaagcaaattgagtcagacaggcagaacctcctcaaggaggtggataacctctacaacatcgagatcctgcggctccccaaggctctgcgcgagatgaactggcttgactacttcgcccttggaggaaacaaacaggccctggaagaggcggcaacagctgacctggatatcaccgaaataaacaaactaacagcagaagctattcagacacccctgaaatctgccaaaacacgaaaggtaatacaggtagatgaaatgatagtggaagaggaagaagaagaagaaaatgaacgtaagaatcttcaaactgcaagagtcaaaaggtgtcctccatccaagaagagaactcagtccatacaaggaaaaggaaaagggaaaaggtcaagccgtgctaacactgttaccccagccgtgggccgattggaggtgtccatggtcaaaccaactccaggcctgacacccaggtttgactcaagggtcttcaagacccctggcctgcgtactccagcagcaggagagcggatttacaacatctcagggaatggcagccctcttgctgacagcaaagagatcttcctcactgtgccagtgggcggcggagagagcctgcgattattggccagtgacttgcagaggcacagtattgcccagctggatccagaggccttgggaaacattaagaagctctccaaccgtctcgcccaaatctgcagcagcatacggacccacaaatgagacaccaaagttgacaggatggacttttaatgggcacttctgggaccctgaagagacttcttcccttcaggcttattgtttgagtgtgaagttccagagcaaggagccatgttcctctaagggaattcaggaattcagacgtgctagtcccacaccagttaggtagagctgtctgttcaccctcccatcccagctgatcccagtcactgcttgctggggccatgccatggaagcttcccatcagtctcccagctgaatcctccctgctctctgagctgctgccttttgcctcctgcaactcaacatcctcttcaccctgccctgcctgcagttgagggggcgaagaagaaccctgtgttctcaggaagactgcctccaccaccgctacccagagaacctctgcatctggcatttctgctctctatgcttgagaccgggaggtttaggctcagataagtgagctctgggccatgagagggtaggtccagaaggtggggggaactgtacagatcagcagagcaggacagttggcagcagtgacctcagtagggaacatgtccgtctaccctctcgcactcatgacacctccccctaccagccctcctcttcctcctcctcctcctcctgtgggaggtggtcagtgggacttagggatctttcacctgctgtgcccagtagttctgaagtctgcttgtggagcagtgttttatgtttatccctgtttactgaagaccaaatactggtttggagacaacttccatgtcttgctcttctacctccctagttagtggaaatttggataagggaactgtagggcccagattctggaggttttatgtcattggccacagaataactgtctctaagctatccatggtccagtggtccctgccaagtctgtagacttcagagagcacttctctcttatggggttcatgggaacaggggtgggtgtgacttgcttggtggcctcattccatgtgtgcctgtgcctggggcatggactttgttaagcagagtcagcagtgaggtcctcattctccagccagcctctctgccctggagaatcatgtgctatgttctaagaatttgagaactagagtcctcatccccaggcttgaaggcacatggctttctcatgtagggctctctgtggtatttgttattattttgcaacaagaccattttagtaaaacagtcctgttcaagttgtattcttttaagttcttttattctcctttccctgagatttttgtatatattgttctgagtaatggtatctttgagctgattgttctaatcagagctggtacctactttcaataaattctggttttgtgttttcttttgtaaaaaaaaaaaaaaaaaa

SEQ ID NO: 13 - DNA, 1720 bp, Homo sapiens
gtctctcctgtctgaaggccagagcaggctgctaggcctggggccaccactgcccctgggtgctacacccagtgtgctgggtcactgggaacttcctgaagtggtgtcacctgaactgggcccccaaggatggggtgcgggcagtaccgcaggaagaggagcagcccctgtgaagattgagagctgccagaggctctgtgattggctgcggcacgatgacccgcgcacggattggctgcttcgggccggggggccgggcccgggggacagaatccgcccccgaaccttcaaagagggtaccccccggcaggagctggcagacccaggaggtgcgacagacccgcggggcaaacggactggggccaagagccgggagcgcgggcgcaaaggcaccagggcccgcccagggcgccgcgcagcacggccttgggggttctgcgggccttcgggtgcgcgtctcgcctctagccatggggtccgcagcgttggagatcctgggcctggtgctgtgcctggtgggctgggggggtctgatcctggcgtgcgggctgcccatgtggcaggtgaccgccttcctggaccacaacatcgtgacggcgcagaccacctggaaggggctgtggatgtcgtgcgtggtgcagagcaccgggcacatgcagtgcaaagtgtacgactcggtgctggctctgagcaccgaggtgcaggcggcgcgggcgctcaccgtgagcgccgtgctgctggcgttcgttgcgctcttcgtgaccctggcgggcgcgcagtgcaccacctgcgtggccccgggcccggccaaggcgcgtgtggccctcacgggaggcgtgctctacctgttttgcgggctgctggcgctcgtgccactctgctggttcgccaacattgtcgtccgcgagttttacgacccgtctgtgcccgtgtcgcagaagtacgagctgggcgcagcgctgtacatcggctgggcggccaccgcgctgctcatggtaggcggctgcctcttgtgctgcggcgcctgggtctgcaccggccgtcccgacctcagcttccccgtgaagtactcagcgccgcggcggcccacggccaccggcgactacgacaagaagaactacgtctgagggcgctgggcacggccgggcccctcctgccagccacgcctgcgaggcgttggataagcctggggagccccgcatggaccgcggcttccgccgggtagcgcggcgcgcaggctcctcggaacgtccggctctgcgccccgacgcggctcctggatccgctcctgcctgcgcccgcagctgaccttctcctgccactagcccggccctgcccttaacagacggaatgaagtttccttttctgtgcgcggcgctgtttccataggcagagcgggtgtcagactgaggatttcgcttcccctccaagacgctgggggtcttggctgctgccttacttcccagaggctcctgctgacttcggaggggcggatgcagagcccagggcccccaccggaagatgtgtacagctggtctttactccatcggcagggcccgagcccagggaccagtgacttggcctggacctcccggtctcactccagcatctccccaggcaaggcttgtgggcaccggagcttgagagagggcgggagtgggaaggctaagaatctgcttagtaaatggtttgaactctctccaaaaaaaaaaaaaaaaaa

SEQ ID NO: 14 - DNA, 1582 bp, Homo sapiens
gcttaggctgagccgtggccgccacagcccatcgtaatgccgcatggtgcttggcactccagagagccaataggaatgaaagaattcatttgaatcggccaatgccggcgggttagggggcgggggttgaaaaccctataaaggcgtcgatcggccggacaggcggcagcggcggctcctgcagcggtggtcggctgttgggtgtggagtttcccagcgcccctcgggtccgaccctttgagcgttctgctccggcgccagcctacctcgctcctcggcgccatgaccacaaccaccaccttcaagggagtcgaccccaacagcaggaatagctcccgagttttgcggcctccaggtggtggatccaatttttcattaggttttgatgaaccaacagaacaacctgtgaggaagaacaaaatggcctctaatatctttgggacacctgaagaaaatcaagcttcttgggccaagtcagcaggtgccaagtctagtggtggcagggaagacttggagtcatctggactgcagagaaggaactcctctgaagcaagctccggagacttcttagatctgaagaaaatgtggacacagacttgccaggcagcctggggcagagtgaagagaagcccgtgcctgctgcgcctgtgcccagcccggtggccccggccccagtgccatccagaagaaatccccctggcggcaagtccagcctcgtcttgggttagctctgactgtcctgaacgctgtcgttctgtctgtttcctccatgcttgtgaactgcacaacttgagcctgactgtacatctcttggatttgtttcattaaaaagaagcactttatgtactgctgtcttttttttttttcttttgaagaacaggtttctctctgtccttgactcttgggtctgtgggccatggcatgagtgttttctagtagtagattggagggaaagctttgtgacacttagtactgtgtttttaagaagaaataatttggttccagatgtgttagaggatcttttgtactgaggtttttaacactttacttgggtttaccaagcctcaactggacagaccataaacagtccacaggcaccgttcctgccaggccccaacccacagggagtctctccgcagagccttcttggtgttgccctaacttgccagtggcctttgctcagagcctcctcctgtgacatgtgaacaatgaagaggcctgcgcctcctgccttgccgcctgcaaagcaaagaaactgccttttattttttaaccttaaaaagtagccagatagtaacaagactggctggctgatgagcaaagcctttgctctcacgcagaggaaggcttggatgtacaatgaaactgcctggaactaaaagcagtgaagcaagggaggcaatcacactgaagcgggtcttcctccaggaacggggtcccacaggcgtgttgttttaaataacctgatgctgtgtgcatgatgctggtgcttgaccatgaaaggaaagtctcatccttaaaatgtgttgtacttcacaatcctggactgttgcttcaagtaaacaatatccacattttgaaa

SEQ ID NO: 15 - DNA, 1696 bp, Homo sapiens
gcttaggctgagccgtggccgccacagcccatcgtaatgccgcatggtgcttggcactccagagagccaataggaatgaaagaattcatttgaatcggccaatgccggcgggttagggggcgggggttgaaaaccctataaaggcgtcgatcggccggacaggcggcagcggcggctcctgcagcggtggtcggctgttgggtgtggagtttcccagcgcccctcgggtccgaccctttgagcgttctgctccggcgccagcctacctcgctcctcggcgccatgaccacaaccaccaccttcaagggagtcgaccccaacagcaggaatagctcccgagacacggggtcttgccatgttgcccaggctggtcttgaactcctaggctcaagtgatgatcctgccttggcctcctagggtgctgggattacagagttttgcggcctccaggtggtggatccaatttttcattaggttttgatgaaccaacagaacaacctgtgaggaagaacaaaatggcctctaatatctttgggacacctgaagaaaatcaagcttcttgggccaagtcagcaggtgccaagtctagtggtggcagggaagacttggagtcatctggactgcagagaaggaactcctctgaagcaagctccggagacttcttagatctgaagggagaaggtgatattcatgaaaatgtggacacagacttgccaggcagcctggggcagagtgaagagaagcccgtgcctgctgcgcctgtgcccagcccggtggccccggccccagtgccatccagaagaaatccccctggcggcaagtccagcctcgtcttgggttagctctgactgtcctgaacgctgtcgttctgtctgtttcctccatgcttgtgaactgcacaacttgagcctgactgtacatctcttggatttgtttcattaaaaagaagcactttatgtactgctgtcttttttttttttcttttgaagaacaggtttctctctgtccttgactcttgggtctgtgggccatggcatgagtgttttctagtagtagattggagggaaagctttgtgacacttagtactgtgtttttaagaagaaataatttggttccagatgtgttagaggatcttttgtactgaggtttttaacactttacttgggtttaccaagcctcaactggacagaccataaacagtccacaggcaccgttcctgccaggccccaacccacagggagtctctccgcagagccttcttggtgttgccctaacttgccagtggcctttgctcagagcctcctcctgtgacatgtgaacaatgaagaggcctgcgcctcctgccttgccgcctgcaaagcaaagaaactgccttttattttttaaccttaaaaagtagccagatagtaacaagactggctggctgatgagcaaagcctttgctctcacgcagaggaaggcttggatgtacaatgaaactgcctggaactaaaagcagtgaagcaagggaggcaatcacactgaagcgggtcttcctccaggaacggggtcccacaggcgtgttgttttaaataacctgatgctgtgtgcatgatgctggtgcttgaccatgaaaggaaagtctcatccttaaaatgtgttgtacttcacaatcctggactgttgcttcaagtaaacaatatccacattttgaaa

SEQ ID NO: 16 - DNA, 1582 bp, Homo sapiens; identical to SEQ ID NO: 14

SEQ ID NO: 17 - DNA, 3824 bp, Homo sapiens
ggaagcgcagagcaggttcaaacacagacggcgggtgaacatggcgtcctcgacttggtctgagacgtgataggcctgccttctggttgaagatgtggcgagtgaaaaaactgagcctcagcctgtcgccttcgccccagacgggaaaaccatctatgagaactcctctccgtgaacttaccctgcagcccggtgccctcaccaactctggaaaaagatcccccgcttgctcctcgctgaccccatcactgtgcaagctggggctgcaggaaggcagcaacaactcatctccagtggattttgtaaataacaagaggacagacttatcttcagaacatttcagtcattcctcaaagtggctagaaacttgtcagcatgaatcagatgagcagcctctagatccaattccccaaattagctctactcctaaaacgtctgaggaagcagtagacccactgggcaattatatggttaaaaccatcgtccttgtaccatctccactggggcagcaacaagacatgatatttgaggcccgtttagataccatggcagagacaaacagcatatctttaaatggacctttgagaacagacgatctggtgagagaggaggtggcaccctgcatgggagacaggttttcagaagttgctgctgtatctgagaaacctatctttcaggaatctccgtcccatctcttagaggagtctccaccaaatccctgttctgaacaactacattgctccaaggaaagcctgagcagtagaactgaggctgtgcgtgaggacttagtaccttctgaaagtaacgccttcttgccttcctctgttctctggctttccccttcaactgccttggcagcagatttccgtgtcaatcatgtggacccagaggaggaaattgtagagcatggagctatggaggaaagagaaatgaggtttcccacacatcctaaggagtctgaaacagaagatcaagcacttgtctcaagtgtggaagatattctgtccacatgcctgacaccaaatctagtagaaatggaatcccaagaagctccaggcccagcagtagaagatgttggtaggattcttggctctgatacagagtcttggatgtccccactggcctggctggaaaaaggtgtaaatacctccgtcatgctggaaaatctccgccaaagcttatcccttccctcgatgcttcgggatgctgcaattggcactacccctttctctacttgctcggtggggacttggtttactccttcagcaccacaggaaaagagtacaaacacatcccagacaggcctggttggcaccaagcacagtacttctgagacagagcagctcctgtgtggccggcctccagatctgactgccttgtctcgacatgacttggaagataacctgctgagctctcttgtcattctggaggttctctcccgccagcttcgggactggaagagccagctggctgtccctcacccagaaacccaggacagtagcacacagactgacacatctcacagtgggataactaataaacttcagcatcttaaggagagccatgagatgggacaggccctacagcaggccagaaatgtcatgcaatcatgggtgcttatctctaaagagctgatatccttgcttcacctatccctgttgcatttagaagaagataagactactgtgagtcaggagtctcggcgtgcagaaacattggtctgttgctgttttgatttgctgaagaaattgagggcaaagctccagagcctcaaagcagaaagggaggaggcaaggcacagagaggaaatggctctcagaggcaaggatgcggcagagatagtgttggaggctttctgtgcacacgccagccagcgcatcagccagctggaacaggacctagcatccatgcgggaattcagaggccttctgaaggatgcccagacccaactggtagggcttcatgccaagcaagaagagctggttcagcagacagtgagtcttacttctaccttgcaacaagactggaggtccatgcaactggattatacaacatggacagctttgctgagtcggtcccgacaactcacagagaaactcacagtcaagagccagcaagccctgcaggaacgtgatgtggcaattgaggaaaagcaggaggtttctagggtgctggaacaagtctctgcccagttagaggagtgcaaaggccaaacagaacaactggagttggaaaacagtcgtctagcaacagatctccgggctcagttgcagattctggccaacatggacagccagctaaaagagctacagagtcagcatacccattgtgcccaggacctggctatgaaggatgagttactctgccagcttacccagagcaatgaggagcaggctgctcaatggcaaaaggaagagatggcactaaaacacatgcaggcagaactgcagcagcaacaagctgtcctggccaaagaggtgcgggacctgaaagagaccttggagtttgcagaccaggagaatcaggttgctcacctggagctgggtcaggttgagtgtcaattgaaaaccacactggaagtgctccgggagcgcagcttgcagtgtgagaacctcaaggacactgtagagaacctaacggctaaactggccagcaccatagcagataaccaggagcaagatctggagaaaacacggcagtactctcaaaagctagggctgctgactgagcaactacagagcctgactctctttctacagacaaaactaaaggagaagactgaacaagagacccttctgctgagtacagcctgtcctcccacccaggaacaccctctgcctaatgacaggaccttcctgggaagcatcttgacagcagtggcagatgaagagccagaatcaactcctgtgcccttgcttggaagtgacaagagtgctttcacccgagtagcatcaatggtttcccttcagcccgcagagaccccaggcatggaggagagcctggcagaaatgagtattatgactactgagcttcagagtctttgttccctgctacaagagtctaaagaagaagccatcaggactctgcagcgaaaaatttgtgagctgcaagctaggctgcaggcccaggaagaacagcatcaggaagtccagaaggcaaaagaagcagacatagagaagctgaaccaggccttgtgcttgcgctacaagaatgaaaaggagctccaggaagtgatacagcagcagaatgagaagatcctagaacagatagacaagagtggcgagctcataagccttagagaggaggtgacccaccttacccgctcacttcggcgtgcggagacagagaccaaagtgctccaggaggccctggcaggccagctggactccaactgccagcctatggccaccaattggatccaggagaaagtgtggctctctcaggaggtggacaaactgagagtgatgttcctggagatgaaaaatgagaaggaaaaactcatgatcaagttccagagccatagaaatatcctagaggagaaccttcggcgctctgacaaggagttagaaaaactagatgacattgttcagcatatttataagaccctgctctctattccagaggtggtgaggggatgcaaagaactacagggattgctggaatttctgagctaagaaactgaaagccagaatctgcttcacctctttttacctgcaatacccccttaccccaataccaagaccaactggcatagagccaactgagataaatgctatttaaataaagtgtatttaatgaatttctccaaaaaaaaaaaaaaaaaa

SEQ ID NO: 18 - DNA, 4224 bp, Homo sapiens
gcgaaattcaagctccaaactctaagctccaagctccaagctccaagctccaagctccaaactcccgccggggtaactggaacccaatccgagggtcatggaggcatcccgaaggtttccggaagccgaggccttgagcccagagcaggctgctcattacctaagatatgtgaaagaggccaaagaagcaactaagaatggagacctggaagaagcatttaaacttttcaatttggcaaaggacatttttcccaatgaaaaagtgctgagcagaatccaaaaaatacaggaagccttggaggagttggcagaacagggagatgatgaatttacagatgtgtgcaactctggcttgctactttatcgagaactgcacaaccaactctttgagcaccagaaggaaggcatagctttcctctatagcctgtatagggatggaagaaaaggtggtatattggctgatgatatgggattagggaagactgttcaaatcattgctttcctttccggtatgtttgatgcatcacttgtgaatcatgtgctgctgatcatgccaaccaatcttattaacacatgggtaaaagaattcatcaagtggactccaggaatgagagtcaaaacctttcatggtcctagcaaggatgaacggaccagaaacctcaatcggattcagcaaaggaatggtgttattatcactacataccaaatgttaatcaataactggcagcaactttcaagctttaggggccaagagtttgtgtgggactatgtcatcctcgatgaagcacataaaataaaaacctcatctactaagtcagcaatatgtgctcgtgctattcctgcaagtaatcgcctcctcctcacaggaaccccaatccagaataatttacaagaactatggtccctatttgattttgcttgtcaagggtccctgctgggaacattaaaaacttttaagatggagtatgaaaatcctattactagagcaagagagaaggatgctaccccaggagaaaaagccttgggatttaaaatatctgaaaacttaatggcaatcataaaaccctattttctcaggaggactaaagaagacgtacagaagaaaaagtcaagcaacccagaggccagacttaatgaaaagaatccagatgttgatgccatttgtgaaatgccttccctttccaggaaaaatgatttaattatttggatacgacttgtgcctttacaagaagaaatatacaggaaatttgtgtctttagatcatatcaaggagttgctaatggagacgcgctcacctttggctgagctaggtgtcttaaagaagctgtgtgatcatcctaggctgctgtctgcacgggcttgttgtttgctaaatcttgggacattctctgctcaagatggaaatgagggggaagattccccagatgtggaccatattgatcaagtaactgatgacacattgatggaagaatctggaaaaatgatattcctaatggacctacttaagaggctgcgagatgagggacatcaaactctggtgttttctcaatcgaggcaaattctaaacatcattgaacgcctcttaaagaataggcactttaagacattgcgaatcgatgggacagttactcatcttttggaacgagaaaaaagaattaacttattccagcaaaataaagattactctgtttttctgcttaccactcaagtaggtggtgtcggtttaacattaactgcagcaactagagtggtcatttttgaccctagctggaatcctgcaactgatgctcaagctgtggatagagtttaccgaattggacaaaaagagaatgttgtggtttataggctaatcacttgtgggactgtagaggaaaaaatatacagaagacaggttttcaaggactcattaataagacaaactactggtgaaaaaaagaaccctttccgatattttagtaaacaagaattaagagagctctttacaatcgaggatcttcagaactctgtaacccagctgcagcttcagtctttgcatgctgctcagaggaaatctgatataaaactagatgaacatattgcctacctgcagtctttggggatagctggaatctcagaccatgatttgatgtacacatgtgatctgtctgttaaagaagagcttgatgtggtagaagaatctcactatattcaacaaagggttcagaaagctcaattcctcgttgaattcgagtctcaaaataaagagttcctgatggaacaacaaagaactagaaatgagggggcctggctaagagaacctgtatttccttcttcaacaaagaagaaatgccctaaattgaataaaccacagcctcagccttcacctcttctaagtactcatcatactcaggaagaagatatcagttccaaaatggcaagtgtagtcattgatgatctgcccaaagagggtgagaaacaagatctctccagtataaaggtgaatgttaccaccttgcaagatggtaaaggtacaggtagtgctgactctatagctactttaccaaaggggtttggaagtgtagaagaactttgtactaactcttcattgggaatggaaaaaagctttgcaactaaaaatgaagctgtacaaaaagagacattacaagaggggcctaagcaagaggcactgcaagaggatcctctggaaagttttaattatgtacttagcaaatcaaccaaagctgatattgggccaaatttagatcaactaaaggatgatgagattttacgtcattgcaatccttggcccattatttccataacaaatgaaagtcaaaatgcagaatcaaatgtatccattattgaaatagctgatgacctttcagcatcccatagtgcactgcaggatgctcaagcaagtgaggccaagttggaagaggaaccttcagcatcttcaccacagtatgcatgtgatttcaatcttttcttggaagactcagcagacaacagacaaaatttttccagtcagtctttagagcatgttgagaaagaaaatagcttgtgtggctctgcacctaattccagagcagggtttgtgcatagcaaaacatgtctcagttgggagttttctgagaaagacgatgaaccagaagaagtagtagttaaagcaaaaatcagaagtaaagctagaaggattgtttcagatggcgaagatgaagatgattcttttaaagatacctcaagcataaatccattcaacacatctctctttcaattctcatctgtgaaacaatttgatgcttcaactcccaaaaatgacatcagtccaccaggaaggttcttttcatctcaaatacccagtagtgtaaataagtctatgaactctagaagatctctggcttctaggaggtctcttattaatatggttttagaccacgtggaggacatggaggaaagacttgacgacagcagtgaagcaaagggtcctgaagattatccagaagaaggggtggaggaaagcagtggcgaagcctccaagtatacagaagaggatccttccggagaaacactgtcttcagaaaacaagtccagctggttaatgacgtctaagcctagtgctctagctcaagagacctctcttggtgcccctgagcctttgtctggtgaacagttggttggttctccccaggataaggcggcagaggctacaaatgactatgagactcttgtaaagcgtggaaaagaactaaaagagtgtggaaaaatccaggaggccctaaactgcttagttaaagcgcttgacataaaaagtgcagatcctgaagttatgctcttgactttaagtttgtataagcaacttaataacaattgagaatgtaacctgtttattgtattttaaagtgaaactgaatatgagggaatttttgttcccataattggattctttgggaacatgaagcattcaggcttaaggcaagaaagatctcaaaaagcaacttctgccctgcaacgccccccactccatagtctggtattctgagcactagcttaatatttcttcacttgaatattcttatattttaggcatattctataaatttaactgtgttgtttcttggaaagttttgtaaaattattctggtcattcttaattttactctgaaagtgatcatctttgtatataacagttcagataagaaaattaaagttacttttctcaagtgttttcaaaaaaaaaaaaaaaaa

SEQ ID NO: 19 - DNA, 4495 bp, Homo sapiens
gccggcgacgtcacgcggccgttacggcgctcaggcgtctcgacgcgcgcgatttaaaaccagctcaggagacgccaaggaaagatgggacctcccggcccagcactgccagccacaatgaataactcttcttcagagacgcgaggacacccccacagtgcctcctctccttcagagcgtgtgttcccgatgcccctgcccaggaaggcgcctctcaatattcctggcaccccagtcctcgaagactttcctcagaatgacgatgagaaggagcggctgcagcggaggcgctcgagggtctttgatctgcagttcagcactgactcacctcgcttattggcctccccctccagcaggagtattgacatttcagctactatccccaagtttacaaacacgcagattacggaacattactccacctgtatcaaactgtccactgaaaataaaatcactaccaagaatgcttttggtttgcacttgattgattttatgtcagagattcttaaacagaaagacaccgaaccaaccaactttaaagtggctgcgggtactctggatgccagcaccaagatctatgctgtgcgcgtggatgccgtccatgccgatgtatacagagtccttggggggctgggcaaagatgcaccgtctttggaagaagtagaaggccatgttgctgatggaagtgctactgaaatgggaacaaccaaaaaggctgtaaagccaaagaagaagcacttacacagaactattgagcagaacataaacaacctcaatgtctccgaagcagatcggaagtgtgagattgatcccatgtttcagaagacagcagcctcatttgatgagtgcagcacagcaggggtgtttctgtccactctccactgccaggactacagaagtgaactgctgtttccctctgatgtccagactctctccacgggagaacctctcgagttgccagagttaggttgtgtagaaatgacagatttaaaagcgcccttgcagcagtgtgcagaagatcgccagatctgcccttccctggccgggttccagtttacacagtgggacagtgaaacacataatgagtctgtgtcggccctggtagacaagtttaagaagaatgaccaggtatttgacatcaatgctgaagttgacgagagtgactgtggagacttccccgatgggtccctgggggatgactttgatgccaacgatgaacctgaccacaccgcagttggggatcatgaagagttcaggagctggaaggagccctgccaggttcagagctgccaggaagaaatgatttcccttggggatggagacatcaggaccatgtgcccccttctgtctatgaaacctggagaatattcttatttcagtcctcggaccatgtcgatgtgggctggcccggatcactggcgctttaggcctcgacgcaaacaagatgctccttcccaatcagaaaacaaaaagaagagtacaaaaaaagattttgaaattgactttgaagatgatattgactttgatgtatattttagaaaaacaaaggctgctactattctgaccaagtccactttggagaaccagaattggagagctaccacccttcctacagatttcaactacaatgttgacactctggtccagcttcacctcaaaccaggcaccaggttacttaagatggcccagggccatagggtagagactgagcattatgaagaaattgaagactatgattacaacaaccctaacgacacctccaacttttgccctggattacaggctgctgacagtgatgatgaagatttggatgacttatttgtgggacctgttgggaactctgacctctcaccttatccttgccatccacctaagacagcacaacagaatggtgacactccagaagcccaaggattagacatcacaacatatggggagtcaaacttggtagctgagcctcagaaggtaaataaaattgaaattcactatgccaagactgccaaaaagatggacatgaagaaactgaagcagagcatgtggagtctgctgacagcgctctccggaaaggaggcagatgcagaggcaaaccacagggaagctggaaaagaagcggccctggcagaagtggctgacgagaagatgcttagcgggctcacgaaggacctgcagaggagcctgccccctgtcatggctcagaacctctccatacctctggcttttgcctgtctcctacatttagccaatgaaaagaatctaaaactggaaggaacagaggacctctctgatgttcttgtgaggcaaggagattgagttcactatggagaagtcagcagcaggaggcccatcccttactcagttgccgggacatccccagtctcgggggaagaagatgccatgggcttatacccaggctgtagccaactaccaacgtgcctgtttgtttgttgctctttccttctctccatcatagtctgggtgccagcgccctgaagctccgtgctcaactgattaaactttactgccctatggtgaccatctaggagaggggagggcagagggggtgagggtactattctggattgagaaaacctatatccattctttatatcaatgtatagttttagtctcctaaattgatctgttattttccaaactattctcttgtagaaaattttccagtgggcacttaatggtgcccttgaagaacttcctaatccatgtacataaaatacatcatatgtacacttataaatgtatatagaatgctcaaaaataaaattcttaataatagaactggcaaaatatttgagtgtccactagatgagtatcagacctagtccttacccttagggggatgcagtcctggttgttatccaggatacacacctgtcagtataaggcagaagatgcctaagggccaagatggtttgcctcggaggagaatggaagagagagattgctgactggacattcagatgcaagactgggtcctgcttaaatcccaggattctgctggagggagctgatagtgatacttgtcccttctgtacattgcttcatgtagccttctcagcatccctaggagaaacttactattgtgactctcatgttggaggaggaaacggacacccaaggtagaggaacttgcaaaagggcagccggcaaactgtcaggggtggcctgagcctggcaatctgcctccagagtctgctctcggccattgtgctatgtgctacctggataggtcatacaggctcagcagtgggtggagagcagtgctcagatttgtccatctccacagaatgcagcacacacacaaatgtacaagttcttcccctaacctcagaggaataggggaattaactttgcttgcaatttggaacaatattatagatgttgatccaagtagttctgttactggctggtcctggatctctgccagaacacccgtcatcattgactggctaaatagagatcttggatataggccagaagcagtgaagtatataattggaaattgctcctgataataacttccttcttagccaaaaaccacacaaaacaaaaataatcccctccccacaggaatatgctttccaaattgtgtccaaaacattacctgctctgttatattgagaaggttagagacttcagagcatgcttagaaaaagcagtggtgccacaggtgagactccacactctgtcttgctggggctgaagcctccatcactttcccaggccaggttagtgctgggcttcttgctttccttctattcctgagagtagaactggctaagcccattccttccctcagtcagccccacttctctatagtgggttctgggggtggggggctgaattaccagtaaaactagaaagattgggaccaagtgcagtggcccacacctgtaaatcctagcgctttgaaaggaagaggcaggaggattgcttgaagtcaggagttcaagaccagcctgggcaaaatagaaccccatctttaaaaaaaaagtttaaaaattagccaggtacggaggtgtgtgcctgtaatcccagctactcagaaggctgaggtgggataatcacttgagcccaggagtttgaggctgcagtgagctgtgatcacactactgcattccagccaggacaacagagtgagatcctatctcttaaacaaaaaaaaaaactggcgagttcaataccaacttctacaatgaaatccccttccccccacaaccctgcttctcctaagtttccctcattacatggttgctgtgggctatgtgtgctgtggtctgaatgtttgtgtctaaaattcacatgttggtattaagagatagggcctttgggaggtgattaggttatgagggcagatccctcgtgaatgggattagtgctcttataaaagaggcctgaggaagcttgttcgttcctcttgcccttctgccatgtaaggatgcaatgagaaggcaccatctgtgagcaaggagcccctcac

SEQ ID NO: 20 - DNA, 2554 bp, Homo sapiens
acaaggcagcctcgctcgagcgcaggccaatcggctttctagctagagggtttaactcctatttaaaaagaagaacctttgaattctaacggctgagctcttggaagacttgggtccttgggtcgcaggtgggagccgacgggtgggtagaccgtgggggatatctcagtggcggacgaggacggcggggacaaggggcggctggtcggagtggcggagcgtcaagtcccctgtcggttcctccgtccctgagtgtccttggcgctgccttgtgcccgcccagcgcctttgcatccgctcctgggcaccgaggcgccctgtaggatactgcttgttacttattacagctagagggtctcactccattgcccaggccagagtgcggggatatttgataagaaacttcagtgaaggccgggcgcggtggctcatgcccgtaatcccagcattttcggaggccgaggctggagtgcaatggtgtgatctcagctcactgcaacctctgcttcctgggtttaagtgattctcctgcctcagcctcccgagtagctgggattacaggcatcatggaccgatctaaagaaaactgcatttcaggacctgttaaggctacagctccagttggaggtccaaaacgtgttctcgtgactcagcaatttccttgtcagaatccattacctgtaaatagtggccaggctcagcgggtcttgtgtccttcaaattcttcccagcgcattcctttgcaagcacaaaagcttgtctccagtcacaagccggttcagaatcagaagcagaagcaattgcaggcaaccagtgtacctcatcctgtctccaggccactgaataacacccaaaagagcaagcagcccctgccatcggcacctgaaaataatcctgaggaggaactggcatcaaaacagaaaaatgaagaatcaaaaaagaggcagtgggctttggaagactttgaaattggtcgccctctgggtaaaggaaagtttggtaatgtttatttggcaagagaaaagcaaagcaagtttattctggctcttaaagtgttatttaaagctcagctggagaaagccggagtggagcatcagctcagaagagaagtagaaatacagtcccaccttcggcatcctaatattcttagactgtatggttatttccatgatgctaccagagtctacctaattctggaatatgcaccacttggaacagtttatagagaacttcagaaactttcaaagtttgatgagcagagaactgctacttatataacagaattggcaaatgccctgtcttactgtcattcgaagagagttattcatagagacattaagccagagaacttacttcttggatcagctggagagcttaaaattgcagattttgggtggtcagtacatgctccatcttccaggaggaccactctctgtggcaccctggactacctgccccctgaaatgattgaaggtcggatgcatgatgagaaggtggatctctggagccttggagttctttgctatgaatttttagttgggaagcctccttttgaggcaaacacataccaagagacctacaaaagaatatcacgggttgaattcacattccctgactttgtaacagagggagccagggacctcatttcaagactgttgaagcataatcccagccagaggccaatgctcagagaagtacttgaacacccctggatcacagcaaattcatcaaaaccatcaaattgccaaaacaaagaatcagctagcaaacagtcttaggaatcgtgcagggggagaaatccttgagccagggctgccatataacctgacaggaacatgctactgaagtttattttaccattgactgctgccctcaatctagaacgctacacaagaaatatttgttttactcagcaggtgtgccttaacctccctattcagaaagctccacatcaataaacatgacactctgaagtgaaagtagccacgagaattgtgctacttatactggttcataatctggaggcaaggttcgactgcagccgccccgtcagcctgtgctaggcatggtgtcttcacaggaggcaaatccagagcctggctgtggggaaagtgaccactctgccctgaccccgatcagttaaggagctgtgcaataaccttcctagtacctgagtgagtgtgtaacttattgggttggcgaagcctggtaaagctgttggaatgagtatgtgattctttttaagtatgaaaataaagatatatgtacagacttgtattttttctctggtggcattcctttaggaatgctgtgtgtctgtccggcaccccggtaggcctgattgggtttctagtcctccttaaccacttatctcccatatgagagtgtgaaaaataggaacacgtgctctacctccatttagggatttgcttgggatacagaagaggccatgtgtctcagagctgttaagggcttatttttttaaaacattggagtcatagcatgtgtgtaaactttaaatatgcaaataaataagtatctatgtctaaaaaaa